(19) United States
(12) Patent Application Publication
(10) Pub. No.: US 2006/0040981 A1
(43) Pub. Date: Feb. 23, 2006

(54) Use of phosphodiesterase-4 inhibitors as enhancers of cognition

(76) Inventors: Daniel Dube, St. Lazare (CA); Michel Gallant, Montreal (CA); Patrick Lacombe, Montreal (CA); Yves Girard, Lle-Bizard (CA); Dwight Macdonald, Lle-Bizard (CA); Richard Friesen, Kirkland (CA); Yves Ducharme, Montreal (CA); Bernard Cote, Lle Perrot (CA); Marc Blouin, St. Lazare (CA); Evelyn Martins, Vaudreuil-Dorion (CA); Daniel Guay, Lle Perrot (CA); Pierre Hamel, Laval (CA); Mario Girard, St. Lazare (CA); Richard Frenette, Laval (CA); Sebastien Laliberte, Lle Perrot (CA); Annette Robichaud, Ville Mont-Royal (CA); Anthony Mastracchio, Edison, NJ (US); Helene Perrier, Burlingame, CA (US)

Correspondence Address: MERCK AND CO., INC, P O BOX 2000, RAHWAY, NJ, 07065-0907, US

(21) Appl. No.: 10/536,250

(22) PCT Filed: Nov. 19, 2003
(86) PCT No.: PCT/CA03/01799

§ 371 Date: May 20, 2005

Related U.S. Application Data
(60) Provisional application No. 60/428,541, filed on Nov. 22, 2002.

(51) Int. Cl. A61K 31/4709; A61K 31/47

(52) U.S. Cl. 514/311; 514/314

(57) ABSTRACT

The present invention is directed to a method of enhancing cognition in a healthy subject comprising administering a safe cognition enhancing amount of a phosphodiesterase-4 inhibitor. In particular, this invention is directed to a method of enhancing memory, learning, retention, recall, awareness and judgement in health subjects comprising administering a safe and effective amount of a phosphodiesterase-4 inhibitor.

### BACKGROUND OF THE INVENTION

1. Field of the Invention

The present invention is directed to a method of enhancing cognition in a healthy subject comprising administering a safe cognition enhancing amount of a phosphodiesterase-4 inhibitor. In particular, this invention is directed to a method of enhancing memory, learning, retention, recall, awareness and judgement in health subjects comprising administering a safe and effective amount of a phosphodiesterase-4 inhibitor.

2. Related Background

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′, 5′ cyclic nucleotides to 5′ nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel anti-asthmatic and anti-inflammatory compounds. PDE-4 is known to exist as at lease four isoenzymes, each of which is encoded by a distinct gene. Each of the four known PDE4 gene products is believed to play varying roles in allergic and/or inflammatory responses. Thus, it is believed that inhibition of PDE4, particularly the specific PDE4 isoforms that produce detrimental responses, can beneficially affect allergy and inflammation symptoms. It would be desirable to provide novel compounds and compositions that inhibit PDE4 activity.

A major concern with the use of PDE4 inhibitors is the side effect of emesis which has been observed for several candidate compounds as described in C. Bumouf et al., (“Bumouf”), Ann. Rep. In Med. Chem., 33:91-109 (1998). B. Hughes et al., Br. J. Pharmacol., 118:1183-1191 (1996); M. J. Perry et al., Cell Biochem. Biophys., 29:113-132 (1998); S. B. Christensen et al., J. Med. Chem., 41:821-835 (1998); and Burnouf describe the wide variation of the severity of the undesirable side effects exhibited by various compounds. As described in M. D. Houslay et al., Adv. In Pharmacol., 44:225-342 (1998) and D. Spina et al., Adv. In Pharmacol., 44:33-89 (1998), there is great interest and research of therapeutic PDE4 inhibitors.

International Patent Publication WO9422852 describes quinolines as PDE4 inhibitors. International Patent Publication WO9907704 describes 1-aryl-1,8-naphthylidin-4-one derivatives as PDE4 inhibitors.

A. H. Cook, et al., J. Chem. Soc., 413-417 (1943) describes gamma-pyridylquinolines. Other quinoline compounds are described in Kei Manabe et al., J. Org. Chem., 58 (24):6692-6700 (1993); Kei Manabe et al., J. Am. Chem. Soc., 115 (12):5324-5325 (1993); and Kei Manabe et al., J. Am. Chem. Soc., 114 (17):6940-6941 (1992).

Compounds that include ringed systems are described by various investigators as effective for a variety of therapies and utilities. For example, International Patent Publication No. WO 98/25883 describes ketobenzamides as calpain inhibitors, European Patent Publication No. EP 811610 and U.S. Pat. Nos. 5,679,712, 5,693,672 and 5,747,541 describe substituted benzoylguanidine sodium channel blockers, U.S. Pat. No. 5,736,297 describes ring systems useful as a photosensitive composition.

U.S. Pat. Nos. 5,491,147, 5,608,070, 5,622,977, 5,739,144, 5,776,958, 5,780,477, 5,786,354, 5,798,373, 5,849,770, 5,859,034, 5,866,593, 5,891,896, and International Patent Publication WO 95/35283 describe PDE4 inhibitors that are tri-substituted aryl or heteroaryl phenyl derivatives. U.S. Pat. No. 5,580,888 describes PDE4 inhibitors that are styryl derivatives. U.S. Pat. No. 5,550,137 describes PDE4 inhibitors that are phenylaminocarbonyl derivatives. U.S. Pat. No. 5,340,827 describes PDE4 inhibitors that are phenylcarboxamide compounds. U.S. Pat. No. 5,780,478 describes PDE4 inhibitors that are tetra-substituted phenyl derivatives. International Patent Publication WO 96/00215 describes substituted oxime derivatives useful as PDE4 inhibitors. U.S. Pat. No. 5,633,257 describes PDE4 inhibitors that are cyclo(alkyl and alkenyl)phenyl-alkenyl (aryl and heteroaryl) compounds.

### SUMMARY OF THE INVENTION

The present invention is directed to a method of enhancing cognition in a healthy subject comprising administering a safe cognition enhancing amount of a phosphodiesterase-4 inhibitor. In particular, this invention is directed to a method of enhancing memory, learning, retention, recall, awareness and judgement in health subjects comprising administering a safe and effective amount of a phosphodiesterase-4 inhibitor.

### DETAILED DESCRIPTION OF THE INVENTION

In one aspect the invention is directed to a method of enhancing cognition in healthy subjects comprising administering a safe cognition enhancing amount of compound of Examples 1A through 34D, or a parmaceutically salt thereof, to the health subject. The compounds of Examples 1A through 34D are:
6-isopropyl-8-(3-{(Z/E)-2-[4-(methylsulfonyl)phenyl]-2-phenylethenyl}phenyl)quinoline;
6-isopropyl-8-{3-[(E/Z)-2-[4-(methylsulfonyl)phenyl]-2-(1,3-thiazol-2-yl)ethenyl]phenyl}quinoline;
6-isopropyl-8-(3-{(E)-2-(1-methyl-1H-imidazol-2-yl)-2-[4-(methylsulfonyl)phenyl]ethenyl)phenyl)quinoline;
6-isopropyl-8-(3-{(Z/E)-2-(4-fluorophenyl)-2-[4-(methylsulfonyl)phenyl]ethenyl}phenyl)quinoline;
2-(2-{(E/Z)-2-[3-(6-isopropyl-8-quinolinyl)phenyl]-1-[4-(methylsulfonyl)phenyl]ethenyl}-1,3-thiazol-5-yl)-2-propanol;
2-[8-(3-1 (E/Z)-2-[5-(1-hydroxy-1-methylethyl)-1,3-thiazol-2-yl]-2-[4-(methylsulfonyl)phenyl]ethenyl)phenyl)-6-quinolinyl]-2-methylpropanenitrile;
2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;
6-[1-(methylsulfonyl)ethyl]-8-{3-[(E)-2-[4-(methylsulfonyl)phenyl]-2-(1,3-thiazol-2-yl)ethenyl]phenyl}quinoline;
6-[1-methyl-1-(methylsulfonyl)ethyl]-8-(3-[(E)-2-[4-(methylsulfonyl)phenyl]-2-(1,3-thiazol-2-yl)ethenyl]phenyl}quinoline;
8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;
8-(3-{(Z)-2-(1-methyl-1H-imidazol-2-yl)-2-[4-(methylsulfonyl)phenyl]ethenyl}phenyl)-6-[1-methyl-1-(methylsulfonyl)ethyl)quinoline;
6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;
(E/Z)-3-{3-[6-(1-cyano-1-methylethyl)-8-quinolinyl]phenyl}-N-isopropyl-2-[4-(methylsulfonyl)phenyl]-2-propenamide;
8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;
(5-{(E)-2-(3-(6-[1-methyl-1-(methylsulfonyl)ethyl]-8-quinolinyl}phenyl)-1-[4-(methylsulfonyl)phenyl]ethenyl)-1,2,4-oxadiazol-3-yl)methanol;
(E)-N-isopropyl-3-(3-{6-[1-methyl-1-(methylsulfonyl)ethyl]-8-quinolinyl}phenyl)-2-[4-(methylsulfonyl)phenyl]-2-propenamide;
(E)-3-{3-[6-(1-cyano-1-methylethyl)-8-quinolinyl]phenyl}-2-[4-(methylsulfonyl)phenyl]-2-propenoic acid;
2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;
(E)-3-{3-[6-(1-cyano-1-methylethyl)-8-quinolinyl]phenyl}-2-[4-(methylsulfonyl)phenyl]-2-propenamide;
(E)-N-(tert-butyl)-3-{3-[6-(1-cyano-1-methylethyl)-8-quinolinyl]phenyl}-2-[4-(methylsulfonyl)phenyl]-2-propenamide;
(E)-3-[3-(6-isopropyl-8-quinolinyl)phenyl]-2-[4-(methylsulfonyl)phenyl]-2-propenoic acid;
6-isopropyl-8-(3-{(E)-2-(3-methyl-1,2,4-oxadiazol-5-yl)-2-[4-(methylsulfonyl)phenyl]ethenyl}phenyl)quinoline;
(E)-3-(3-(6-[1-methyl-1-(methylsulfonyl)ethyl]-8-quinolinyl)phenyl)-2-[4-(methylsulfonyl)phenyl]-1-(1-pyrrolidinyl)-2-propen-1-one;
(E)-N-cyclopropyl-3-(3-{6-[1-methyl-1-(methylsulfonyl)ethyl]-8-quinolinyl}phenyl)-2-[4-(methylsulfonyl)phenyl]-2-propenamide;
(E)-N-(tert-butyl)-3-(3-{6-[1-methyl-1-(methylsulfonyl)ethyl]-8-quinolinyl) phenyl)-2-[4-(methylsulfonyl)phenyl]-2-propenamide;
8-{3-[2,2-bis(4-chlorophenyl)vinyl]phenyl}-6-isopropylquinoline;
6-isopropyl-8-(3-[(E/Z)-2-(6-methyl-3-pyridinyl)-2-[4-(methylsulfonyl)phenyl]ethenyl)phenyl)quinoline;
6-isopropyl-8-(3-{(E/Z)-2-(5-methyl-2-pyridinyl)-2-[4-(methylsulfonyl)phenyl]ethenyl}phenyl)quinoline;
8-(3-{2,2-bis[4-(methylsulfonyl)phenyl]vinyl}phenyl)-6-isopropylquinoline;
2-methyl-2-[8-(3-{(E/Z)-2-(5-methyl-2-pyridinyl)-2-[4-(methylsulfonyl)phenyl]ethenyl}phenyl)-6-quinolinyl-]propanenitrile;
2-[8-(3-{2,2-bis[4-(methylsulfonyl)phenyl]vinyl}phenyl)-6-quinolinyl]-2-methylpropanenitrile;
2-methyl-2-(8-{3-[(E)-2-[4-(methylsulfonyl)phenyl]-2-(2-pyridinyl)ethenyl]phenyl}-6-quinolinyl)propanenitrile;
6-[1-methyl-1-(methylsulfonyl)ethyl]-8-(3-((E/Z)-2-(5-methyl-2-pyridinyl)-2-[4-(methylsulfonyl)phenyl]ethenyl}phenyl)quinoline;
2-(6-{(E)-2-(3-{6-[1-methyl-1-(methylsulfonyl)ethyl]-8-quinolinyl}phenyl)-1-[4-(methylsulfonyl)phenyl]ethenyl}-3-pyridinyl)-2-propanol;
2-[8-(3-{(E/Z)-3-(dimethylamino)-2-[4-(methylsulfonyl)phenyl]prop-1-enyl}phenyl)quinolin-6-yl]-2-methylpropanenitrile;
(±)-4-{2-[3,4-Bis(difluoromethoxy)phenyl]-2-{5-[2-(1-hydroxy-1-methyl)ethyl]thiazolyl}ethyl}pyridine N-oxide;
chiral 4-{2-[3,4-Bis(difluoromethoxy)phenyl]-2-{5-[2-(1-hydroxy-1-methyl)ethyl]thiazolyl}ethyl}pyridine N-oxide;
(±/±)-4-{2-[3,4-Bis(difluoromethoxy)phenyl]-2-[5-(2-(1-hydroxy-2,2,2-trifluoro)ethyl)thiazolyl]ethyl}pyridine;
(±/±)-4-{2-[3,4-Bis(difluoromethoxy)phenyl]-2-[5-(2-(1-hydroxy-2,2,2-trifluoro)ethyl)thiazolyl]ethyl}pyridine n-oxide;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine n-oxide;
(±/±)-4-{2-[3,4-Bis(difluoromethoxy)phenyl]-2-{5-[2-(1-hydroxy-1-trifluoromethyl)ethyl]thiazolyl}ethyl}pyridine N-oxide;
(±/±)-4-{2-[3,4-Bis(difluoromethoxy)phenyl]-2-[5-(2-phenylmethanol)thiazolyl]ethyl}pyridine n-oxide;
(±/±)-4-{2-[3,4-Bis(difluoromethoxy)phenyl]-2-[5-(2-(1-hydroxy-1-phenyl)ethyl)thiazolyl]ethyl}pyridine n-oxide;
(±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-phenyl)propyl)thiazolyl]ethyl}pyridine n-oxide;
(±/±)-4-{2-[3,4-Bis(difluoromethoxy)phenyl]-2-[5-(2-cyclohexylmethanol)thiazolyl]ethyl}pyridine;
(±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine n-oxide;
(±/±)-4-{2-[3,4-Bis(difluoromethoxy)phenyl]-2-[5-(2-(1-hydroxy-1-(4-ethyl)phenyl)ethyl)thiazolyl]ethyl}pyridine n-oxide;
(±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine n-oxide;
(±/±)-4-{2-(3,4-Bis(difluoromethoxy)phenyl]-2-[5-(2-(1-hydroxy-1-(4-fluoro)phenyl)ethyl)thiazolyl]ethyl}pyridine n-oxide;
(±/±)-4-{2-[3,4-Bis(difluoromethoxy)phenyl]-2-[5-(2-(1-hydroxy-1-(4-Fluoro)phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine n-oxide;
(±)-4-{2-[3,4-Bis(difluoromethoxy)phenyl]-2-{5-[2-(1-hydroxy)cyclobutyl]thiazolyl}ethyl}pyridine N-oxide;
(±)-4-{2-[3,4-Bis(difluoromethoxy)phenyl]-2-{5-[2-(1-hydroxy)cyclohexyl]thiazolyl}ethyl}pyridine N-oxide;
(±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine n-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine;
chiral 3-{2-[(3-cyclobutyloxy-4-difluoromethoxy)phenyl]-2-{5-[2-(1-hydroxy-1-methyl)ethyl]thiazolyl}ethyl}pyridine n-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine n-oxide;
chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
(±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
(±)-3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
N-Isopropyl-1-[3-(3-acetylphenyl)phenyl]-1,4-dihydro[1,8]naphthyridin-4-one-3-carboxamide; N-(2,6-Dichloropyridin-4-yl)-1-[3-(3-acetylphenyl)phenyl]-1,4-dihydro[1,8]naphthyridin-4-one-3-carboxamide;
N-Isopropyl-1-[3-(4-n-propylphenyl)phenyl]-1,4-dihydro[1,8]naphthyridin-4-one-3-carboxamide;
N-Isopropyl-1-[3-(4-acetylphenyl)phenyl]-1,4-dihydro[1,8]naphthyridin-4-one-3-carboxamide
N-Isopropyl-1-[3-(2-methylphenyl)phenyl]-1,4-dihydro[1,8]naphthyridin-4-one-3-carboxamide;
N-Isopropyl-N-methyl-1-[3-(4-acetylphenyl)phenyl]-1,4-dihydro[1,8]naphthyridin-4-one-3-carboxamide;
N-Isopropyl-1-[3-(pyridin-3-yl)phenyl]-1,4-dihydro[1,8]naphthyridin-4-one-3-carboxamide;
N-Isopropyl-1-[3-(indol-5-yl)phenyl]-1,4-dihydro[1,8]naphthyridin-4-one-3-carboxamide;
N-tert-Butyl-1-[3-(4-acetylphenyl)phenyl]-1,4-dihydro[1,8]naphthyridin-4-one-3-carboxamide;
N-(2,6-Dichloropyridin-4-yl)-1-[3-(pyridin-3-yl)phenyl]-1,4-dihydro[1,8]naphthyridin-4-one-3-carboxamide;
N-Isopropyl-1-{3-[4-(4-tertbutyloxycarbonylpiperazin-1-yl)phenyl]-phenyl}-1,4-dihydro[1,8]naphthyridin-4-one-3-carboxamide;
N-Isopropyl-1-[3-(quinolin-3-yl)phenyl]-1,4-dihydro[1,8]naphthyridin-4-one-carboxamide;
N-Isopropyl-1-[3-(pyrimidin-5-yl)phenyl]-1,4-dihydro[1,8]naphthyridin-4-one-carboxamide;
N-Cyclopropyl-1-[3-(pyridin-3-yl)phenyl]-1,4-dihydro[1,8]naphthyridin-4-one-3-carboxamide;
N-Isopropyl-1-[3-(5-methylthiopyridin-3-yl)phenyl-1,4-dihydro[1,8]naphthyridin-4-one-3-carboxamide;
N-Cyclopropyl-1-[3-(4-hydroxymethylphenyl)phenyl]-1,4-dihydro[1,8]naphthyridin-4-one-3-carboxamide;
N-Cyclopropyl-1-[3-(pyridin-4-yl)phenyl]-1,4-dihydro[1,8]naphthyridin-4-one-3-carboxamide;
N-Cyclopropyl-1-[3-(4-ethylthiophenyl)phenyl]-1,4-dihydro[1,8]naphthyridin-4-one-3-carboxamide;
N-Cyclopropyl-1-[3-(3-thienyl)phenyl]-1,4-dihydro[1,8]naphthyridin-4-one-3-carboxamide;
N-Cyclopropyl-1-[3-(4-sulfamoylphenyl)phenyl]-1,4-dihydro[1,8]naphthyridin-4-one-3-carboxamide;
N-Isopropyl-1-[3-(3-ethoxyphenyl)phenyl]-1,4-dihydro[1,8]naphthyzidin-4-one-3-carboxamide;
N-Isopropyl-1-[3-(4-methylthiophenyl)phenyl]-1,4-dihydro[1,8]naphthyridin-4-one-3-carboxamide;
N-Isopropyl-1-[3-(3-acetyl-4-hydroxyphenyl)phenyl]-1,4-dihydro[1,8]naphthyridin-4-one-3-carboxamide;
N-Isopropyl-1-[3-(5-carboethoxypyridin-3-yl)phenyl]-1,4-dihydro[1,8]naphthyridin-4-one-3-carboxamide;
N-Isopropyl-1-{3-[5-(1-hydroxy-1-methylethyl)pyridin-3-yl]phenyl}-1,4-dihydro[1,8]naphthyridin-4-one-3-carboxamide;
N-Isopropyl-1-{3-[6-(2-methylpropyl)pyridin-3-yl]phenyl}-1,4-dihydro[1,8]naphthyridin-4-one-3-carboxamide;
N-Isopropyl-1-[3-(5-acetylpyridin-3-yl)phenyl]-1,4-dihydro[1,8]naphthyridin-4-one-3-carboxamide;
N-Isopropyl-1-[3-(6-methylpyridin-3-yl)phenyl]-1,4-dihydro[1,8]naphthyridin-4-one-3-carboxamide;
N-Cyclopropyl-1-[3-(1-oxidopyrinidin-5-yl) phenyl]-1,4-dihydro[1,8]naphthyridin-4-one-3-carboxamide;
1-{3-[6-(1-Hydroxy-1-methylethyl)-1-oxidopyridin-3-yl]phenyl}-1,4-dihydro[1,8]naphthyridin-4-one-3-carboxamide;
N-Isopropyl-1-{3-[4-(pyridin-3-yl)phenyl]phenyl}-1,4-dihydro[1,8]naphthyridin-4-one-3-carboxamide;
N-Cyclopropyl-1-[3-(5-methylsulfonylpyridin-3-yl)]phenyl]-1,4-dihydro[1,8]naphthyridin-4-one-3-carboxamide;
N-Cyclopropyl-1-{3-[4-(1-hydroxy-1-methylethyl)-1-oxidopyridin-2-yl]phenyl}-1,4-dihydro[1,8]naphthyridin-4-one-3-carboxamide;
N-Cyclopropyl-1-{3-[5-(1-hydroxy-1-methylethyl)pyridin-2-yl]phenyl}1,4-dihydro[1,8]naphthyridin-4-one-3-carboxamide;
N-Cyclopropyl-1-{3-[3-(1-hydroxy-1-methylethyl)pyridin-4-yl]phenyl}-1,4-dihydro[1,8]naphthyridin-4-one-3-carboxamide;
N-Cyclopropyl-1-{3-[3-(1-hydroxy-1-methylethyl)-1-oxidopyridin-4-yl]phenyl}-1,4-dihydro[1,8]naphthyridin-4-one-3-carboxamide;
N-Cyclopropyl-1-[3-(6-isopropylsulfonylpyridin-3-yl)]phenyl]-1,4-dihydro[1,8]naphthyridin-4-one-3-carboxamide;
N-Cyclopropyl-1-[3-(6-methoxypyridin-3-yl)phenyl]-1,4-dihydro[1,8]naphthyridin-4-one-3-carboxamide;
N-Cyclopropyl-1-[3-(6-methylpyridin-3-yl)phenyl]-1,4-dihydro[1,8]naphthyridin-4-one-3-carboxamide;
N-Cyclopropyl-1-{3-[6-(2,2,2-trifluoroethoxy)pyridin-3-yl]phenyl}-1,4-dihydro[1,8]naphthyridin-4-one-3-carboxamide;
N-Cyclopropyl-1-[3-(5-bromopyridin-3-yl)phenyl]-1,4-dihydro[1,8]naphthyridin-4-one-3-carboxamide;
N-Cyclopropyl-1-[3-(6-benzyloxypyridin-3-yl)phenyl]-1,4-dihydro[1,8]naphthyridin-4-one-3-carboxamide;
N-Cyclopropyl-1-{3-[6-dicyclopropyl(hydroxy)methyl-1-oxidopyridin-3-yl]phenyl}-1,4-dihydro[1,8]naphthyridin-4-one-3-carboxamide;
N-Cyclopropyl-1-{3-[5-(1-hydroxy-1-methylethyl)-1-oxidopyridin-2-yl]phenyl}-1,4-dihydro[1,8]naphthyridin-4-one-3-carboxamide;
N-Cyclopropyl-1-{3-[6-(1-hydroxy-1-methylethyl)pyridin-3-yl]phenyl}-1,4-dihydro[1,8]naphthyridin-4-one-3-carboxamide;
N-Isobutyl-1-{3-[6-(1-hydroxy-1-methylethyl)pyridin-3-yl]phenyl}-1,4-dihydro[1,8]naphthyridin-4-one-3-carboxamide;
N-Cyclopropyl-1-{5-bromo-3-[6-(1-hydroxy-1-methylethyl)pyridin-3-yl]phenyl}-1,4-dihydro[1,8]naphthyridin-4-one-3-carboxamide;
N-Cyclopropyl-1-{3-[6-(1-hydroxy-1-methylethyl)pyridin-2-yl]phenyl}-1,4-dihydro[1,8]naphthyridin-4-one-3-carboxamide;
N-Isopropyl-1-[3-(4-methylsulfonylphenyl)phenyl]-1,4-dihydro[1,8]naphthyridin-4-one-3-carboxamide;
N-Cyclopropyl-1-[3-(6-methylsulfonylpyridin-3-yl)phenyl]-1,4-dihydro[1,8]naphthyridin-4-one-3-carboxamide;
N-Isopropyl-1-[3-(5-methylsulfonylpyridin-3-yl)phenyl]-1,4-dihydro[1,8]naphthyridin-4-one-3-carboxamide;
N-Cyclopropyl-1-[3-(4-ethylsulfonylphenyl)phenyl]-1,4-dihydro[1,8]naphthyridin-4-one-3-carboxamide;
N-Cyclopropyl-1-[3-(4-ethylsulfinylphenyl)phenyl]-1,4-dihydro[1,8]naphthyridin-4-one-3-carboxamide;
N-Isopropyl-1-{3-[4-(1-oximidoethyl)phenyl]phenyl}-1,4-dihydro[1,8]naphthyridin-4-one-3-carboxamide;
N-Isopropyl-1-{3-[4-(4-piperazin-1-yl)phenyl]-phenyl}-1,4-dihydro[1,8]naphthyridin-4-one-3-carboxamide;
N-Cyclopropyl-1-[3-(4-methylsulfonylmethylphenyl)phenyl]-1,4-dihydro[1,8]naphthyridin-4-one-3-carboxamide;
N-Cyclopropyl-1-[3-(1,6-dihydro-6-oxopyridin-3-yl)phenyl]-1,4-dihydro[1,8]naphthyridin-4-one-3-carboxamide;
N-Cyclopropyl-1-[[3-{5-[6-(1-hydroxy-1-methylethyl)pyridin-3-yl]pyridin-3-yl}phenyl]]-1,4-dihydro[1,8]naphthyridin-4-one-3-carboxamide;
N-Isopropyl-1-[3-(1-oxidopyridin-3-yl)phenyl]-1,4-dihydro[1,8]naphthyridin-4-one-3-carboxamide;
N-(2,6-Dichloropyridin-4-yl)-1-[3-(1-oxidopyridin-3-yl)phenyl]-1,4-dihydro[1,8]naphthyridin-4-one-3-carboxamide;
N-Isopropyl-1-[3-(5-carboethoxy-1-oxidopyridin-3-yl)phenyl]-1,4-dihydro[1,8]naphthyridin-4-one-3-carboxamide;
N-Isopropyl-1-{3-[5-(1-hydroxy-1-methylethyl)-1-oxidopyridin-3-yl]phenyl}-1,4-dihydro[1,8]naphthyridin-4-one-3-carboxamide;
N-Isopropyl-1-{3-[6-(2-methylpropyl)-1-oxidopyridin-3-yl]phenyl}-1,4-dihydro[1,8]naphthyridin-4-one-3-carboxamide;
N-Isopropyl-1-[3-(6-methyl-1-oxidopyridin-3-yl)phenyl]-1,4-dihydro[1,8]naphthyridin-4-one-3-carboxamide;
N-Cyclopropyl-1-[3-(1-oxidopyridin-3-yl)phenyl]-1,4-dihydro[1,8]naphthyridin-4-one-3-carboxamide;
N-Cyclopropyl-1-{3-[6-(1-hydroxy-1-methylethyl)-1-oxidopyridin-3-yl]phenyl}-1,4-dihydro[1,8]naphthyridin-4-one-3-carboxamide;
N-Cyclopropyl-1-[3-(1-oxidopyridin-4-yl)phenyl]-1,4-dihydro[1,8]naphthyridin-4-one-3-carboxamide;
N-Cyclopropyl-1-[3-(5-bromo-1-oxidopyridin-3-yl)phenyl]-1,4-dihydro[1,8]naphthyridin-4-one-3-carboxamide;
N-Cyclopropyl-1-[[3-{5-[6-(1-hydroxy-1-methylethyl)-1-oxidopyridin-3-yl]pyridin-3-yl}phenyl]]-1,4-dihydro[1,8]naphthyridin-4-one-3-carboxamide;
N-Cyclopropyl-1-[[3-{5-[6-(1-hydroxy-1-methylethyl)pyridin-3-yl]-1-oxidopyridin-3-yl}phenyl]]-1,4-dihydro[1,8]naphthyridin-one-3-carboxamide;
N-Cyclopropyl-1-[[3-{5-[6-(1-hydroxy-1-methylethyl)-1-oxidopyridin-3-yl]-1-oxidopyridin-3-yl}phenyl)]-1,4-dihydro[1,8]naphthyridin-4-one-3-carboxamide;
N-Isopropyl-1-[3-(1-oxidoquinolin-3-yl)phenyl]-1,4-dihydro[1,8]naphthyridin-4-one-carboxamide;
N-Isobutyl-1-{3-[6-(1-hydroxy-1-methylethyl)-1-oxidopyridin-3-yl]phenyl}-1,4-dihydro[1,8]naphthyridin-4-one-3-carboxamide;
N-Cyclopropyl-1-[3-(6-methyl-1-oxidopyridin-3-yl)]phenyl]-1,4-dihydro[1,8]naphthyridin-4-one-3-carboxamide;
N-Cyclopropyl-1-[3-(6-methylsulfonyl-1 oxidopyridin-3-yl)phenyl]-1,4-dihydro[1,8]naphthyridin-4-one-3-carboxamide;
N-Cyclopropyl-1-{5-bromo-3-[6-(1-hydroxy-1-methylethyl)-1-oxidopyridin-3-yl]phenyl}-1,4-dihydro[1,8]naphthyridin-4-one-3-carboxamide;
N-Cyclopropyl-1-{3-[6-(1,2-dihydroxy-1-methylethyl)-1-oxidopyridin-3-yl]phenyl}-1,4-dihydro[1,8]naphthyridin-4-one-3-carboxamide;
N-Isopropyl-1-[3-(phenylethynyl)phenyl]-1,4-dihydro[1,8]naphthyridin-4-one-3-carboxamide;
N-Isopropyl-1-[3-(2-pyridinylethynyl)phenyl]-1,4-dihydro[1,8]naphthyridin-4-one-3-carboxamide;
N-Isopropyl-1-[3-(4-pyridinylethynyl)phenyl]-1,4-dihydro[1,8]naphthyridin-4-one-3-carboxamide;
N-Isopropyl 1-[3-(1-oxido-4-pyridinylethynyl)phenyl]-1,4-dihydro[1,8]naphthyridin-4-one-3-carboxamide;
N-Isopropyl-1-(3-ethynylphenyl)-1,4-dihydro[1,8]naphthyridin-4-one-3-carboxamide;
N-Cyclopropyl-1-(3-ethynylphenyl)-1,4-dihydro[1,8]naphthyridin-4-one-3-carboxamide;
N-Isopropyl-1-[3-(3-pyridinylethynyl)phenyl]-1,4-dihydro[1,8]naphthyridin-4-one-3-carboxamide;
N-Isopropyl-1-[3-(1-oxido-3-pyridinylethynyl)phenyl-1,4-dihydro[1,8]naphthyridin-4-one-3-carboxamide;
N-Cyclopropyl-1-[3-(3-pyridinylethynyl)phenyl]-1,4-dihydro[1,8]naphthyridin-4-one-3-carboxamide;
N-Isopropyl-1-[3-(3-hydroxy-3-methylbut-1-ynyl)phenyl]-1,4-dihydro[1,8]naphthyridin-4-one-3-carboxamide;
N-Cyclopropyl-1-[3-(3-hydroxy-3-methylbut-1-ynyl)phenyl]-1,4-dihydro[1,8]naphthyridin-4-one-3-carboxamide;
N-Isopropyl-1-[3-(1-hydroxycyclopentyl)ethynylphenyl]-1,4-dihydro[1,8]naphthyridin-4-one-3-carboxamide;
N-Isopropyl-1-[3-(1-hydroxycyclopropyl)ethynylphenyl]-1,4-dihydro[1,8]naphthyridin-4-one-3-carboxamide;
N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide;
N-Isopropyl-1-[3-(3-hydroxy-3-phenylbut-1-ynyl)phenyl]-1,4-dihydro[1,8]naphthyridin-4-one-3-carboxamide;
N-Cyclopropyl-1-[3-(1-oxido-3-pyridinylethynyl)phenyl]-1,4-dihydro[1,8]naphthyridin-4-one-3-carboxamide;
N-Isopropyl-1-[3-(3-amino-3-ethylpent-1-ynyl)phenyl]-1,4-dihydro[1,8]naphthyridin-4-one-3-carboxamide; or
N-Cyclopropyl-1-[3-(3-amino-3-ethylpent-1-ynyl)phenyl]-1,4-dihydro[1,8]naphthyridin-4-one-3-carboxamide;
N-Isopropyl-1-[3-(3-quinolinylethynyl)phenyl]-1,4-dihydro(1,8]naphthyridin-4-one-3-carboxamide;
N-Isopropyl-1-[3-(1-oxido-3-quinolinylethynyl)phenyl]-1,4-dihydro[1,8]naphthyridin-4-one-3-carboxamide;
N-Isopropyl-1-[3-(cyclopropylethynyl)phenyl]-1,4-dihydro[1,8]naphthyridin-4-one-3-carboxamide;
N-Isopropyl-1-[3-(6-amino-3-pyridinylethynyl)phenyl]-1,4-dihydro[1,8]naphthyridin-4-one-3-carboxamide;
N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[6-(1-hydroxy-1-methylethyl)-3-pyridinylethynyl]phenyl}-1,4-dihydro[1,8]naphthyridin-4-one-3-carboxamide;
N-Isopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[4-(1-hydroxy-1-methylethyl)-2-pyridinylethynyl]phenyl}-1,4-dihydro[1,8]naphthyridin-4-one-3-carboxamide;
N-Isopropyl-1-{3-[5-(1-hydroxy-1-methylethyl)-2-pyridinylethynyl]phenyl}-1,4-dihydro[1,8]naphthyridin-4-one-3-carboxamide;
N-Isopropyl-1-{3-[6-(1-hydroxy-1-methylethyl)-2-pyridinylethynyl]phenyl}-1,4-dihydro[1,8]naphthyridin-4-one-3-carboxamide;
N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[(4-pyridin-3-ylphenyl)ethynyl]phenyl}-1,4-dihydro[1,8]naphthyridin-4-one-3-carboxamide;
N-Isopropyl-1-(3-{[5-(1-hydroxy-1-methylethyl)thien-2-yl]ethynyl}phenyl)-1,4-dihydro[1,8]naphthyridin-4-one-3-carboxamide;
N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide;
1-[3-(1-Oxido-3-pyridinylethynyl)phenyl]-1,4-dihydro[1,8]naphthyridin-4-one-3-carboxamide; and
1-[3-(1-Oxido-3-pyridinylethynyl)phenyl]-1,4-dihydro[1,8]naphthyridin-4-one-3-carboxylic acid,
   or a pharmaceutically acceptable salt thereof.

In another aspect, the invention encompass a method of of enhancing cognition in healthy subjects comprising administering a safe cognition enhancing amount of compound of the compounds below to the healthy subject: 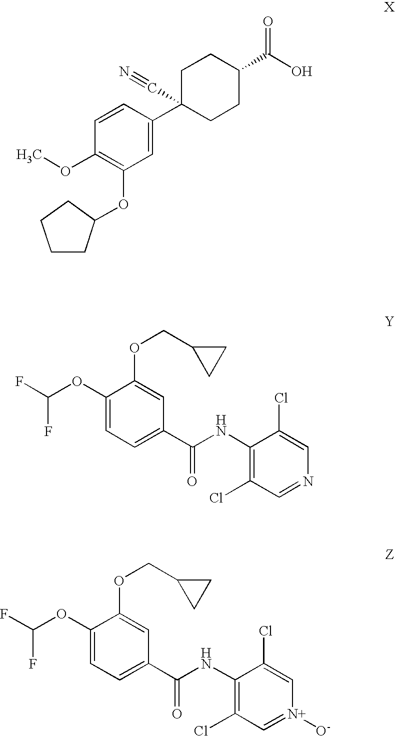
or a pharmaceutically acceptable salt thereof.

Compounds X, Y and Z may be prepared as disclosed in U.S. Pat. No. 5,552,438, U.S. Pat. No. 5,712,298 and U.S. Pat. No. 6,448,274, which are hereby incorporated by reference.

As mentioned above, the present invention is directed to a method of enhancing cognition in a healthy subject comprising administering a safe cognition enhancing amount of a phosphodiesterase-4 inhibitor. In particular, this invention is directed to a method of enhancing memory, learning, retention, recall, awareness and judgement in health subjects comprising administering a safe and cognition enhancing amount of a phosphodiesterase-4 inhibitor. Within this aspect there is a method of enhancing cognition in a healthy subject comprising administering a safe, non-emetic, cognition enhancing amount of a phosphodiesterase-4 inhibitor.

For purposes of this application is defined as a subject with cognition in the normal range for the subjects age or other classification. Cognition of a healthy subject as well as cognition enhancement of the healthy subject is illustrated shown by testing the compounds in the Morris water maze as reported by McNamara and Skelton, Psychobiology, 1993, 21, 101-108. Further details of relevant methodology are described in WO 96/25948. Other assessments for measuring cognition enhancement include, but are not limited to the “T” Maze Test; Radial Arm Maze Test; Delayed Non-Match or Delayed Match Test; Passive Avoidance Procedure; 5 Choice Test, disclosed in WO 01/87281 A2, published Nov. 22, 2001.

For purposes of this specification, classes of healthy subjects includes juveniles, adults and seniors of average cognition; juveniles, adults and seniors of above average cognition; and juveniles, adults and seniors of below average cognition.

For purposes of this specification, juvenile human subjects is defined as a human subject less than 18 years of age. For purposes of this specification, adult human subject is defined as a human subject 18 years of age or older. Within this classification is a human adult 18 to 40 years of age. For purposes of this specification, senior human subjects is defined as a human subject 40 years of age or older. Within this classification is a human subject 55 years of age or older; 65 years of age or older; and 70 years of age or older.

As appreciated by those of skill in the art, beginning at about age 25, the cognition of the healthy human declines at a measurable and reproducible rates, as for example, measured by CAmbridge Neuropsychological Test Automated Battery (CANTAB, de Jager C A, Milwain E, Budge M. Early detection of isolated memory deficits in the elderly: the need for more sensitive neuropsychological tests. Psychol Med 2002 April;32 (3):483-91) or the Cognitive Drug Reseach Battery (CDR, Barker A, Jones R, Simpson P, Wesnes K. (1995). Scopolamine induced cognitive impairment as a predictor of cognitive decline in healthy elderly volunteers. International Journal of Geriatric Psychiatry 10: 1059-1062). Thus, by the time a human subject becomes a senior 40 years of age the decline in cognitive function has declined significant and would benefit from a method of memory enhancement.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

The pharmaceutical compositions of the present invention comprise a compound (or pharmaceutically acceptable salts thereof) as an active ingredient, a pharmaceutically acceptable carrier and optionally other therapeutic ingredients or adjuvants. Such additional therapeutic ingredients include, for example, i) Leukotriene receptor antagonists, ii) Leukotriene biosynthesis inhibitors, iii) corticosteroids, iv) H1 receptor antagonists, v) beta 2 adrenoceptor agonists, vi) COX-2 selective inhibitors, vii) statins, viii) non-steroidal anti-inflammatory drugs (“NSAID”), and ix) M2/M3 antagonists. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.0001 mg/kg to about 50 mg/kg of body weight per day are useful for enhancing cognition or about 0.005 mg to about 2.5 g per patient per day. Alternatively, dosage levels from about 0.001 mg to 10 mg of the compound per kilogram of body weight per day, or alternatively about 0.05 mg to about 500 mg per patient per day.

The amount of active ingredient that may be combined with the carrier materials to produce a single dosage form will vary depending upon the host treated and the particular mode of administration. For example, a formulation intended for the oral administration to humans may conveniently contain from about 0.005 mg to about 2.5 g of active agent, compounded with an appropriate and convenient amount of carrier materia. Unit dosage forms will generally contain between from about 0.005 mg to about 1000 mg of the active ingredient, typically 0.005, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of a compound of the Examples. The compounds or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described, or pharmaceutically acceptable salts thereof, may also be prepared in powder or liquid concentrate form.

Further, the compound of this invention can be utilized in combination with other therapeutic compounds. In particular, the combinations of the PDE4 inhibiting compound of this invention can be advantageously used in combination with i) leukotriene receptor antagonists, ii) Leukotriene biosynthesis inhibitors, iii) COX-2 selective inhibitors, iv) statins, v) NSAIDs, vi) M2/M3 antagonists, vii) corticosteroids, viii) H1 (histamine) receptor antagonists and ix) beta 2 adrenoceptor agonist.

Thus, for example, cognition can be conveniently enhanced with capsules, cachets or tablets each containing 0.005, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg of the active ingredient of the compound of the present application, or a pharmaceutically acceptable salt thereof, administered once, twice, or three times daily.

The abbreviations used herein have the following tabulated meanings. Abbreviations not tabulated below have their meanings as commonly used unless specifically stated otherwise.

| | |
|---|---|
| Ac = | Acetyl |
| Bn = | Benzyl |
| CAMP | cyclic adenosine-3′,5′-monophosphate |
| DBU = | 1,8-diazabicyclo[5.4.0]undec-7-ene |
| DIBAL = | Diisobutylaluminum hydride |
| DMAP = | 4-(dimethylamino)pyridine |
| DMF = | N,N-dimethylfonnamide |
| Et₃N = | Triethylamine |
| GST | Glutathione transferase |
| HMDS | Hexamethyldisilazide |
| LDA = | lithium diisopropylamide |
| m-CPBA = | metachloroperbenzoic acid |
| MMPP = | monoperoxyphthalic acid |
| MPPM = | monoperoxyphthalic acid, magnesium salt 6H₂O |
| Ms = | methanesulfonyl = mesyl = SO₂Me |
| Ms0 = | methanesulfonate = mesylate |
| NSAID = | non-steroidal anti-inflammatory drug |
| o-Tol = | ortho-tolyl |
| OXONE ® = | 2KHSO₅.KHSO₄.K₂SO₄ |
| PCC = | pyridinium chlorochromate |
| PDC = | pyridinium dichromate |
| PDE | Phosphodiesterase |
| Ph = | Phenyl |
| Phe = | Benzenediyl |
| PMB = | para-methoxybenzyl |
| Pye = | Pyridinediyl |
| r.t. = | room temperature |
| Rac. = | Racemic |
| SAM = | aminosulfonyl or sulfonamide or SO₂NH₂ |
| SEM = | 2-(trimethylsilyl)ethoxymethoxy |
| SPA = | scintillation proximity assay |
| TBAF = | tetra-n-butylammonium fluoride |
| Th = | 2- or 3-thienyl |
| TFA = | trifluoroacetic acid |
| TFAA = | trifluoroacetic acid anhydride |
| THF = | Tetrahydrofuran |
| Thi = | Thiophenediyl |
| TLC = | thin layer chromatography |
| TMS-CN = | trimethylsilyl cyanide |
| TMSI | trimethylsilyl iodide |
| Tz = | 1H (or 2H)-tetrazol-5-yl |
| CAN | ceric ammonium nitrate |
| C₃H₅= | Allyl |

ALKYL GROUP ABBREVIATIONS

| | |
|---|---|
| Me = | Methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | Cyclobutyl |
| c-Pen = | cyclopentyl |
| c-Hex = | cyclohexyl |

### Assays Demonstrating Biological Activity

### LPS and FMLP-Induced TNF-α and LTB₄ Assays in Human Whole Blood

Whole blood provides a protein and cell-rich milieu appropriate for the study of biochemical efficacy of anti-inflammatory compounds such as PDE4-selective inhibitors. Normal non-stimulated human blood does not contain detectable levels of TNF-α and LTB₄. Upon stimulation with LPS, activated monocytes express and secrete TNF-α up to 8 hours and plasma levels remain stable for 24 hours. Published studies have shown that inhibition of TNF-α by increasing intracellular cAMP via PDE₄ inhibition and/or enhanced adenylyl cyclase activity occurs at the transcriptional level. LTB₄ synthesis is also sensitive to levels of intracellular cAMP and can be completely inhibited by PDE₄-selective inhibitors. As there is little LTB₄ produced during a 24 hour LPS stimulation of whole blood, an additional LPS stimulation followed by fMLP challenge of human whole blood is necessary for LTB₄ synthesis by activated neutrophils. Thus, by using the same blood sample, it is possible to evaluate the potency of a compound on two surrogate markers of PDE4 activity in the whole blood by the following procedure.

Fresh blood was collected in heparinized tubes by venipuncture from healthy human volunteers (male and female). These subjects had no apparent inflammatory conditions and had not taken any NSAIDs for at least 4 days prior to blood collection. 500 μL aliquots of blood were pre-incubated with either 2 μL of vehicle (DMSO) or 2 μL of test compound at varying concentrations for 15 minutes at 37° C. This was followed by the addition of either 10 μL vehicle (PBS) as blanks or 10 μL LPS (1 μg/mL final concentration, #L-2630 (Sigma Chemical Co., St. Louis, Mo.) from E. coli, serotype 0111:B4; diluted in 0.1% w/v BSA (in PBS)). After 24 hours of incubation at 37° C., another 10 μL of PBS (blank) or 10 μL of LPS (1 μg/mL final concentration) was added to blood and incubated for 30 minutes at 37° C. The blood was then challenged with either 10 μL of PBS (blank) or 10 μL of fMLP (1 μM final concentration, #F-3506 (Sigma); diluted in 1% w/v BSA (in PBS)) for 15 minutes at 37° C. The blood samples were centrifuged at 1500×g for 10 minutes at 4° C. to obtain plasma. A 50/L aliquot of plasma was mixed with 200 μL methanol for protein precipitation and centrifuged as above. The supernatant was assayed for LTB₄ using an enzyme immunoassay kit (#520111 from Cayman Chemical Co., Ann Arbor, Mich.) according to the manufacturer's procedure. TNF-α was assayed in diluted plasma (in PBS) using an ELISA kit (Cistron Biotechnology, Pine Brook, N.J.) according to manufacturer's procedure. IC₅₀ values should be less than about 5 μM, advantageously less than about 2.5 μM. The IC₅₀ values of Examples 1 to 33 ranged from 0.01 μM to 2.4 μM.

### Anti-Allergic Activity In Vivo

Compounds of the invention have been tested for effects on an IgE-mediated allergic pulmonary inflammation induced by inhalation of antigen by sensitized guinea pigs. Guinea pigs were initially sensitized to ovalbumin under mild cyclophosphamide-induced immunosuppression, by intraperitoneal injection of antigen in combinations with aluminum hydroxide and pertussis vaccine. Booster doses of antigen were given two and four weeks later. At six weeks, animals were challenged with aerosolized ovalbumin while under cover of an intraperitoneally administered anti-histamine agent (mepyramine). After a further 48 h, bronchial alveolar lavages (BAL) were performed and the numbers of eosinophils and other leukocytes in the BAL fluids were counted. The lungs were also removed for histological examination for inflammatory damage. Administration of compounds of the Examples (0.001-10 mg/kg i.p. or p.o.), up to three times during the 48 h following antigen challenge, lead to a significant reduction in the eosinophilia and the accumulation of other inflammatory leukocytes.

### Spa Based PDE Activity Assay Protocol

Compounds which inhibit the hydrolysis of cAMP to AMP by the type-IV cAMP-specific phosphodiesterases were screened in a 96-well plate format as follows:

In a 96 well-plate at 30° C. the test compound was added (dissolved in 2 μL DMSO), 188 μL of substrate buffer containing [2,8-³H] adenosine 3′,5′-cyclic phosphate (cAMP, 100 nM to 50 μM), 10 mM MgCl₂, 1 mM EDTA, 50 mM Tris, pH 7.5. The reaction was initiated by the addition of human recombinant PDE4 (the amount was controlled so that ˜10% product was formed in 10 min.). The reaction was stopped after 10 min. by the addition of 1 mg of PDE-SPA beads (Amersham Pharmacia Biotech, Inc., Piscataway, N.J.). The product AMP generated was quantified on a Wallac Microbeta® 96-well plate counter (EG&G Wallac Co., Gaithersburg, Md.). The signal in the absence of enzyme was defined as the background. 100% activity was defined as the signal detected in the presence of enzyme and DMSO with the background subtracted. Percentage of inhibition was calculated accordingly. IC₅₀ value was approximated with a non-linear regression fit using the standard 4-parameter/multiple binding sites equation from a ten point titration.

The IC₅₀ values of Examples 1 to 33 were determined with 100 nM cAMP using the purified GST fusion protein of the human recombinant phosphodiesterase IVa (met-248) produced from a baculovirus/Sf-9 expression system. IC₅₀ values should be less than about 1000 nM, advantageously less than about 250 nM, and even more advantageously less than about 100 nM. The IC₅₀ values of Examples 1 to 33 ranged from 0.1 nM to 90.0 nM.

The Examples that follow are intended as an illustration of certain preferred embodiments of the invention and no limitation of the invention is implied.

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) and reaction times are given for illustration only. Melting points are uncorrected and “d” indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. broad; etc. In addition, “Ar” signifies an aromatic signal. Chemical symbols have their usual meanings; the following abbreviations have also been used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### EXAMPLES

The Examples are comprised of four sub-sets—Example set A, Example set B, Example set C and Example Set D.

### Examples A1 Through A 42

The compounds of Examples 1A through 42A are characterized and prepared as disclosed in U.S. Pat. No. 6,410,563 B1, issued Jun. 25, 2002, which is hereby incorporated by reference.
1A. and 2A. 6-isopropyl-8-(3-{(Z/E)-2-[4-(methylsulfonyl)phenyl]-2-phenylethenyl}phenyl)quinoline;
3A. 6-isopropyl-8-{3-[(E/Z)-2-[4-(methylsulfonyl)phenyl]-2-(1,3-thiazol-2-yl)ethenyl]phenyl}quinoline;
4A. 6-isopropyl-8-(3-{(E)-2-(1-methyl-1H-imidazol-2-yl)-2-[4-(methylsulfonyl)phenyl]ethenyl}phenyl)quinoline;
5A. and 6A. 6-isopropyl-8-(3-{(Z/E)-2-(4-fluorophenyl)-2-[4-(methylsulfonyl)phenyl]ethenyl}phenyl)quinoline;
7A. 2-(2-{(E/Z)-2-[3-(6-isopropyl-8-quinolinyl)phenyl]-1-[4-(methylsulfonyl)phenyl]ethenyl)-1,3-thiazol-5-yl)-2-propanol;
8A. 2-[8-(3-{(E/Z)-2-[5-(1-hydroxy-1-methylethyl)-1,3-thiazol-2-yl]-2-[4-(methylsulfonyl)phenyl]ethenyl}phenyl)-6-quinolinyl]-2-methylpropanenitrile;
9A. 2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;
10A. 6-[1-(methylsulfonyl)ethyl]-8-{3-[(E)-2-[4-(methylsulfonyl)phenyl]-2-(1,3-thiazol-2-yl)ethenyl]phenyl}quinoline;
11A. 6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;
12A. 8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;
13A. 8-(3-{(Z)-2-(1-methyl-1H-imidazol-2-yl)-2-[4-(methylsulfonyl)phenyl]ethenyl}phenyl)-6-[1-methyl-1-(methylsulfonyl)ethyl]quinoline;
14A. and 15. 6-[1-methyl-1-(methylsulfonyl)ethyl]-8-(3-((E/Z)-2-(3-methyl-1,2,4-oxadiazol-5-yl)-2-[4-(methylsulfonyl)phenyl]ethenyl}phenyl)quinoline;
16A. and 17A. (E/Z)-3-{3-[6-(1-cyano-1-methylethyl)-8-quinolinyl]phenyl}-N-isopropyl-2-[4-(methylsulfonyl)phenyl]-2-propenamide;
18A. 8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;
19A. (5-{(E)-2-(3-{6-[1-methyl-1-(methylsulfonyl)ethyl]-8-quinolinyl}phenyl)-1-[4-(methylsulfonyl)phenyl]ethenyl}-1,2,4-oxadiazol-3-yl)methanol;
20A. (E)-N-isopropyl-3-(3-{6-[1-methyl-1-(methylsulfonyl)ethyl]-8-quinolinyl}phenyl)-2-[4-(methylsulfonyl)phenyl]-2-propenamide;
21A. (E)-3-{3-[6-(1-cyano-1-methylethyl)-8-quinolinyl]phenyl}-2-[4-(methylsulfonyl)phenyl]-2-propenoic acid;
22A. 2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;
23A. (E)-3-{3-[6-(1-cyano-1-methylethyl)-8-quinolinyl]phenyl}-2-[4-(methylsulfonyl)phenyl]-2-propenamide;
24A. E)-N-(tert-butyl)-3-{3-[6-(1-cyano-1-methylethyl)-8-quinolinyl]phenyl}-2-[4-(methylsulfonyl)phenyl]-2-propenamide;
25A. (E)-3-[3-(6-isopropyl-8-quinolinyl)phenyl]-2-[4-(methylsulfonyl)phenyl]-2-propenoic acid;
26A. 6-isopropyl-8-(3-{(E)-2-(3-methyl-1,2,4-oxadiazol-5-yl)-2-[4-(methylsulfonyl)phenyl]ethenyl}phenyl)quinoline;
27A. (E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;
28A. (E)-N-cyclopropyl-3-(3-{6-[1-methyl-1-(methyl sulfonyl)ethyl]-8-quinolinyl}phenyl)-2-[4-(methylsulfonyl)phenyl]-2-propenamide;
29A. (E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;
30A. 8-{3-[2,2-bis(4-chlorophenyl)vinyl]phenyl}-6-isopropylquinoline;
31A. and 32A. 6-isopropyl-8-(3-{(E/Z)-2-(6-methyl-3-pyridinyl)-2-[4-(methylsulfonyl)phenyl]ethenyl}phenyl)quinoline;
33A. and 34A. 6-isopropyl-8-(3-{(E/Z)-2-(5-methyl-2-pyridinyl)-2-[4-(methylsulfonyl)phenyl]ethenyl}phenyl)quinoline;
35A. 8-(3-{2,2-bis[4(methylsulfonyl)phenyl]vinyl}phenyl)-6-isopropylquinoline;
36A. and 37A. 2-methyl-2-[8-(3-{(E/Z)-2-(5-methyl-2-pyridinyl)-2-[4-(methylsulfonyl)phenyl]ethenyl}phenyl)-6-quinolinyl]propanenitrile;
38A. 2-[8-(3-{2,2-bis[4-(methylsulfonyl)phenyl]vinyl}phenyl)-6-quinolinyl]-2-methylpropanenitrile;
39A. 2-methyl-2-(8-{3-[(E)-2-[4-(methylsulfonyl)phenyl]-2-(2-pyridinyl)ethenyl]phenyl}-6-quinolinyl)propanenitrile;
40A. and 41A. 6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;
42A. 2-(6-{(E)-2-(3-{6-[1-methyl-1-(methylsulfonyl)ethyl]-8-quinolinyl}phenyl)-1-[4-(methylsulfonyl)phenyl]ethenyl}-3-pyridinyl)-2-propanol;
   The compounds of Examples 1B through 36 are characterized and prepared as disclosed in U.S. Pat. No. 6,399,636 B2, issued Jun. 4, 2002, which is hereby incorporated by reference.

Examples 1B-36B are summarized in the table below:

| Example | R1 | R2 | R3 | R4 | Pyridine | n |
|---|---|---|---|---|---|---|
| 1B | CHF₂ | CHF₂ | CH₃ | CH₃ | 4-Pyr | 1 |
| 2B | CHF₂ | CHF₂ | CH₃ | CH₃ | 4-Pyr | 1 |
| 3B | CHF₂ | CHF₂ | CF₃ | H | 4-Pyr | 0 |
| 4B | CHF₂ | CHF₂ | CF₃ | H | 4-Pyr | 1 |
| 5B | CHF₂ | CHF₂ | CF₃ | CF₃ | 4-Pyr | 0 |
| 6B | CHF₂ | CHF₂ | CF₃ | CF₃ | 4-Pyr | 1 |
| 7B | CHF₂ | CHF₂ | CF₃ | CH₃ | 4-Pyr | 1 |
| 8B | CHF₂ | CHF₂ | Ph | H | 4-Pyr | 1 |
| 9B | CHF₂ | CHF₂ | Ph | CH₃ | 4-Pyr | 1 |
| 10B | CHF₂ | CHF₂ | Ph | CF₃ | 4-Pyr | 1 |
| 11B | CHF₂ | CHF₂ | Ph | Et | 4-Pyr | 1 |
| 12B | CHF₂ | CHF₂ | c-Hex | H | 4-Pyr | 0 |
| 13B | CHF₂ | CHF₂ | c-Hex | CF₃ | 4-Pyr | 1 |
| 14B | CHF₂ | CHF₂ | 4-EtPh | CH₃ | 4-Pyr | 1 |
| 15B | CHF₂ | CHF₂ | 4-EtPh | CF₃ | 4-Pyr | 1 |
| 16B | CHF₂ | CHF₂ | 4-FPh | CH₃ | 4-Pyr | 1 |
| 17B | CHF₂ | CHF₂ | 4-FPh | CF₃ | 4-Pyr | 1 |
| 18B | CHF₂ | CHF₂ | 2-(5-Br)Pyr | CF₃ | 4-Pyr | 1 |
| 19B | CHF₂ | CHF₂ | 3-(6-Br)Pyr | CF₃ | 4-Pyr | 1 |
| 20B | CHF₂ | CHF₂ | —(CH₂)₃— | | 4-Pyr | 1 |
| 21B | CHF₂ | CHF₂ | —(CH₂)₅— | | 4-Pyr | 1 |
| 22B | CHF₂ | c-but | CH₃ | CH₃ | 4-Pyr | 1 |
| 23B | CHF₂ | c-but | CH₃ | CH₃ | 4-Pyr | 1 |
| 24B | CHF₂ | c-but | CF₃ | CF₃ | 4-Pyr | 0 |
| 25B | CHF₂ | c-but | CF₃ | CF₃ | 4-Pyr | 1 |
| 26B | CHF₂ | c-but | CH₃ | CH₃ | 3-Pyr | 0 |
| 27B | CHF₂ | c-but | CH₃ | CH₃ | 3-Pyr | 0 |
| 28B | CHF₂ | c-but | CH₃ | CH₃ | 3-Pyr | 1 |
| 29B | CHF₂ | c-but | CH₃ | CH₃ | 3-Pyr | 1 |
| 30B | CHF₂ | c-but | CF₃ | CF₃ | 3-Pyr | 1 |
| 31B | CHF₂ | c-but | CF₃ | CF₃ | 3-Pyr | 1 |
| 32B | CHF₂ | c-but | CF₃ | CF₃ | 2-Pyr | 1 |
| 33B | CHF₂ | c-pr | CH₃ | CH₃ | 4-Pyr | 1 |
| 34B | CHF₂ | c-pr | CF₃ | CF₃ | 3-Pyr | 1 |
| 35B | CHF₂ | c-pr | CF₃ | CF₃ | 3-Pyr | 1 |
| 36B | CHF₂ | c-pr | CF₃ | CF₃ | 3-Pyr | 1 |

In the table above, “c-but” represents cyclobutyl, “c-pr” represents cyclopropyl, “c-pent” represents cyclopentyl, “c-Hex” represents cyclohexyl, “4-EtPh” represents 4-ethylphenyl, “4-FPh” represents 4-fluorophenyl, “Ph” represents phenyl, “Pyr” represents pyridyl, “2-(5-Br)Pyr” represents 2-(5-bromo)pyridyl, and “3-(6-Br)Pyr” represents 3-(6-bromo)pyridyl.
(±)-4-{2-[3,4-Bis(difluoromethoxy)phenyl]-2-{5-[2-(1-hydroxy-1-methyl)ethyl]thiazolyl}ethyl}pyridine N-oxide;
chiral 4-{2-[3,4-Bis(difluoromethoxy)phenyl]-2-{5-[2-(1-hydroxy-1-methyl)ethyl]thiazolyl}ethyl}pyridine N-oxide;
(±/±)-4-{2-[3,4-Bis(difluoromethoxy)phenyl]-2-[5-(2-(1-hydroxy-2,2,2-trifluoro)ethyl)thiazolyl]ethyl}pyridine;
(±/±)-4-{2-[3,4-Bis(difluoromethoxy)phenyl]-2-[5-(2-(1-hydroxy-2,2,2-trifluoro)ethyl)thiazolyl]ethyl}pyridine n-oxide;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine n-oxide;
(±/±)-4-{2-[3,4-Bis(difluoromethoxy)phenyl]-2-{5-[2-(1-hydroxy-1-trifluoromethyl)ethyl]thiazolyl}ethyl}pyridine N-oxide;
(±/±)-4-{2-[3,4-Bis(difuoromethoxy)phenyl]-2-[5-(2-phenylmethanol)thiazolyl]ethyl}pyridine n-oxide;
(±/±)-4-{2-[3,4-Bis(difluoromethoxy)phenyl]-2-[5-(2-(1-hydroxy-1-phenyl)ethyl)thiazolyl]ethyl}pyridine n-oxide;
(±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-phenyl)propyl)thiazolyl]ethyl}pyridine n-oxide;
(±/±)-4-{2-[3,4-Bis(difluoromethoxy)phenyl]-2-[5-(2-cyclohexylmethanol)thiazolyl]ethyl}pyridine;
(±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine n-oxide;
(±/±)-4-{2-[3,4-Bis(difluoromethoxy)phenyl]-2-[5-(2-(1-hydroxy-1-(4-ethyl)phenyl)ethyl)thiazolyl]ethyl}pyridine n-oxide;
(±/±)-4-(2-[3,4-Bis(difluoromethoxy)phenyl]-2-[5-(2-(1-hydroxy-1-(4-ethyl)phenyl-2,2,2-trifluoro)ethyl)thiazolyl]ethyl)pyridine n-oxide;
(±/±)-4-{2-[3,4-Bis(difluoromethoxy)phenyl]-2-[5-(2-(1-hydroxy-1-(4-fluoro)phenyl)ethyl)thiazolyl]ethyl}pyridine n-oxide;
(±/±)-4-{2-[3,4-Bis(difluoromethoxy)phenyl]-2-[5-(2-(1-hydroxy-1-(4-Fluoro)phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine n-oxide;
(±)-4-{2-[3,4-Bis(difluoromethoxy)phenyl]-2-{5-[2-(1-hydroxy)cyclobutyl]thiazolyl}ethyl}pyridine N-oxide;
(±)-4-{2-[3,4-Bis(difluoromethoxy)phenyl]-2-{5-[2-(1-hydroxy)cyclohexyl]thiazolyl}ethyl}pyridine N-oxide;
(±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine n-oxide;
Chiral 3-{2-[(3-cyclobutyloxy-4-difluoromethoxy)phenyl]-2-{5-[2-(1-hydroxy-1-methyl)ethyl]thiazolyl}ethyl}pyridine;
Chiral 3-{2-[(3-cyclobutyloxy-4-difluoromethoxy)phenyl]-2-{5-[2-(1-hydroxy-1-methyl)ethyl]thiazolyl}ethyl}pyridine n-oxide;
Chiral 3-{2-[(3-cyclobutyloxy-4-difluoromethoxy)phenyl]-2-{5-[2-(1-hydroxy-1-methyl)ethyl]thiazolyl}ethyl}pyridine n-oxide;
Chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
(±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
(±)-3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide; and

chiral 3-{2-[(3-cyclopropyloxy-4-difluoromethoxy)phenyl]-2-{5-[2-(1-hydroxy-1-trifluoromethyl-2,2,2-tifluoro)ethyl]thiazolyl}ethyl}pyridine N-oxide.

TABLE 1C

| | |
|---|---|
| | I |
| 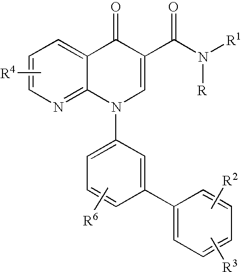 | |

| EX. | R | R¹ | R² | R³ | R⁴ | R⁶ |
|---|---|---|---|---|---|---|
| 1C | H | i-pr | 3-C(O)Me | H | H | H |
| 2C | H | 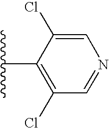 | 3-C(O)Me | H | H | H |
| 3C | H | i-pr | 4-n-propyl | H | H | H |
| 4C | H | i-pr | 4-C(O)Me | H | H | H |
| 5C | H | i-pr | 2-Me | H | H | H |
| 6C | Me | i-pr | 4-C(O)Me | H | H | H |
| 9C | H | t-bu | 4-C(O)Me | H | H | H |
| 11C | H | i-pr | 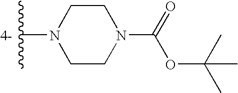 | H | H | H |
| 16C | H | c-pr | 4-CH₂OH | H | H | H |
| 18C | H | c-pr | 4-SEt | H | H | H |
| 20C | H | c-pr | 4-SO₂NH₂ | H | H | H |
| 21C | H | i-pr | 3-OEt | H | H | H |
| 22C | H | i-pr | 4-SMe | H | H | H |
| 23C | H | i-pr | 3-C(O)Me | 4-OH | H | H |
| 49C | H | i-pr | 4-SO₂Me | H | H | H |
| 52C | H | c-pr | 4-SO₂Et | H | H | H |
| 53C | H | c-pr | 4-S(O)Et | H | H | H |
| 54C | H | i-pr | 4-C(═NOH)Me | H | H | H |
| 55C | H | i-pr | 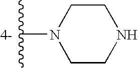 | H | H | H |
| 56C | H | c-pr | 4-CH₂SO₂Me | H | H | H |

TABLE 2

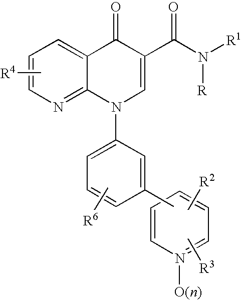

| | | | π- | | | | | |
|---|---|---|---|---|---|---|---|---|
| | | | Posi- | | | | | |
| EX. | R | R¹ | tion | R² | R³ | R⁴ | R⁶ | n |
| 7C | H | i-pr | 3 | H | H | H | H | 0 |
| 10C | H | 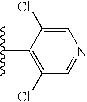 | H | H | H | H | 0 | |
| 14C | H | c-pr | 3 | H | H | H | H | 0 |
| 15C | H | i-pr | 3 | 5-Sme | H | H | H | 0 |
| 17C | H | c-pr | 4 | H | H | H | H | 0 |
| 24C | H | i-pr | 3 | 5-COOEt | H | H | H | 0 |
| 25C | H | i-pr | 3 | 5-CMe₂OH | H | H | H | 0 |
| 26C | H | i-pr | 3 | 6-CH₂CHMe₂ | H | H | H | 0 |
| 27C | H | i-pr | 3 | 5-C(O)Me | H | H | H | 0 |
| 28C | H | i-pr | 3 | 6-Me | H | H | H | 0 |
| 30C | H | H | 3 | 6-CMe₂OH | H | H | H | 1 |
| 32C | H | c-pr | 3 | 5-SO₂Me | H | H | H | 0 |
| 33C | H | c-pr | 2 | 4-CMe₂OH | H | H | H | 1 |
| 34C | H | c-pr | 2 | 5-CMe₂OH | H | H | H | 0 |
| 35C | H | c-pr | 4 | 3-CMe₂OH | H | H | H | 0 |
| 36C | H | c-pr | 4 | 3-CMe₂OH | H | H | H | 1 |
| 37C | H | c-pr | 3 | 6-SO₂I-pr | H | H | H | 0 |
| 38C | H | c-pr | 3 | 6-Ome | H | H | H | 0 |
| 39C | H | c-pr | 3 | 6-Me | H | H | H | 0 |
| 40C | H | c-pr | 3 | 6-OCH₂CF₃ | H | H | H | 0 |
| 41C | H | c-pr | 3 | 5-Br | H | H | H | 0 |
| 42C | H | c-pr | 3 | 6-OCH₂Ph | H | H | H | 0 |
| 43C | H | c-pr | 3 | 6-C(c-pr)₂OH | H | H | H | 0 |
| 44C | H | c-pr | 2 | 5-CMe₂OH | H | H | H | 1 |
| 45C | H | c-pr | 3 | 6-CMe₂OH | H | H | H | 0 |
| 46C | H | i-butyl | 3 | 6-CMe₂OH | H | H | H | 0 |
| 47C | H | c-pr | 3 | 6-CMe₂OH | H | H | 5- | 0 |
| | | | | | | | Br | |
| 48C | H | c-pr | 2 | 6-CMe₂OH | H | H | H | 0 |
| 50C | H | c-pr | 3 | 6-SO₂Me | H | H | H | 0 |
| 51C | H | i-pr | 3 | 5-SO₂Me | H | H | H | 0 |
| 59C | H | i-pr | 3 | H | H | H | H | 1 |
| 60C | H | 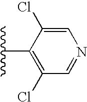 | 3 | H | H | H | H | 1 |
| 61C | H | i-pr | 3 | 5-COOEt | H | H | H | 1 |
| 62C | H | i-pr | 3 | 5-CMe₂OH | H | H | H | 1 |
| 63C | H | i-pr | 3 | 6-CH₂CHMe₂ | H | H | H | 1 |
| 64C | H | i-pr | 3 | 6-Me | H | H | H | 1 |
| 65C | H | c-pr | 3 | H | H | H | H | 1 |
| 66C | H | c-pr | 3 | 6-CMe₂OH | H | H | H | 1 |
| 67C | H | c-pr | 4 | H | H | H | H | 1 |
| 68C | H | c-pr | 3 | 5-Br | H | H | H | 1 |
| 73C | H | i-butyl | 3 | 6-CMe₂OH | H | H | H | 1 |
| 74C | H | c-pr | 3 | 6-Me | H | H | H | 1 |
| 75C | H | c-pr | 3 | 6-SO₂Me | H | H | H | 1 |
| 76C | H | c-pr | 3 | 6-CMe₂OH | H | H | 5- | 1 |
| | | | | | | | Br | |
| 77C | H | c-pr | 3 | 6-CMe(CH2OH)OH | H | H | H | 1 |

TABLE 3

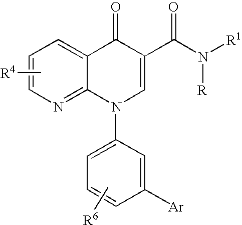

| | | | | | |
|---|---|---|---|---|---|
| Example | R | R¹ | Ar | R⁴ | R⁶ |

| | | | | | |
|---|---|---|---|---|---|
| 8C | H | i-pr | indol-5-yl | H | H |
| 12C | H | i-pr | quinolin-3-yl | H | H |
| 13C | H | i-pr | Pyrimidin-5-yl | H | H |
| 19C | H | c-pr | 3-thienyl | H | H |
| 29C | H | c-pr | 1-oxidopyrimidin-5-yl | H | H |
| 57C | H | c-pr | 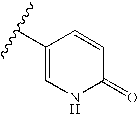 | H | H |
| 72C | H | i-pr | 1-oxidoquinolin-3-yl | H | H |

TABLE 4

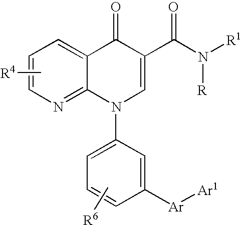

| Ex- | | | | | | |
|---|---|---|---|---|---|---|
| ample | R | R¹ | Ar | Ar¹ | R⁴ | R⁶ |
| 31C | H | i-pr | Ph | 4-(pyridin-3-yl) | H | H |
| 58C | H | c-pr | Pyridin-3-yl | 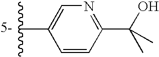 | H | H |
| 69C | H | c-pr | Pyridin-3-yl | 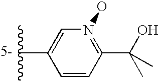 | H | H |
| 70C | H | c-pr | 1- oxidopyridin- 3-yl | 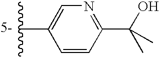 | H | H |
| 71C | H | c-pr | 1- oxidopyridin -3-yl | 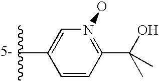 | H | H |

### Example 1C

### N-Isopropyl-1-[3-(3-acetylphenyl)phenyl]-1,4-dihydro[1,8]naphthyridin-4-one-3-carboxamide

### Step 1: Ethyl 3-(3-bromoanilino)-2-(2-chloronicotinoyl) acrylate

A mixture of ethyl 2-chloronicotinoyl acetate (41.1 g, 180.5 mmol), triethyl orthoformate (40.12 g, 271 mmol) and acetic anhydride (92.05 g, 902.5 mmol) was heated at 130° C. for 2.5 hours. The volatile components were distilled off and the residue was co-evaporated twice with xylene. The oily residue was dissolved in methylene chloride (250 mL) and 3-bromoaniline (37.25 g, 216.6 mmol) was added slowly. The resulting solution was stirred at room temperature for 18 hours, and the solvent evaporated away. The resulting crude compound was used as such in the next step.

### Step 2: Ethyl 1-(3-bromophenyl)-1,4-dihydro[1,8]naphthyridin-4-one-3-carboxylate

The crude compound from Step 1 was dissolved in tetrahydrofuran (500 mL), the solution was cooled to 0° C., and sodium hydride (as a 60% dispersion in oil, 9.4 g, 235 mmol) was added in portions. After stirring at 0° for 1 hour, the mixture was allowed to warm up to room temperature. After 2 hours, water (400 mL) was added to the suspension and and the insoluble solid was filtered and washed copiously with water. When dry, the solid was stirred in ether (150 mL) at room temperature for 24 hours and filtered to afford the title compound as a cream-colored solid.

¹H NMR (Acetone-d₆) δ 1.32 (t, 3H), 4.29 (q, 2H), 7.54-7.63 (m, 2H), 7.69 (dd, 1H), 7.78 (dd, 1H), 7.93 (s, 1H), 8.66-8.71 (m, 3H).

### Step 3: 1-(3-Bromophenyl)-1,4-dihydro[1,8]naphthyridin-4-one-3-carboxylic acid

A suspension of ethyl 1-(3-bromophenyl)-1,4-dihydro[1,8]naphthyridin-4-one-3-carboxylate from Step 2 (52.5 g, 140.7 mmol) in a mixture of tetrahydrofuran (400 mL), methanol (400 mL) and 1N aqueous sodium hydroxide (280 mL) was heated at ca 50° C. with stirring for 20 minutes. After cooling, the mixture was diluted with water (300 mL) and 1N aqueous HCl (325 mL) was added. After stirring for 45 minutes, the precipitate was filtered, washed well with water and dried to afford the title acid as a cream-colored solid.

¹H NMR (Acetone-d₆) δ 7.65 (t, 1H), 7.76 (m, 2H), 7.84 (d, 1H), 7.99 (s, 1H), 8.87 (m, 2H), 9.01 (s, 1H).

### Step 4: N-Isopropyl-1-(3-bromophenyl)-1,4-dihydro[1,8]naphthyridin-4-one-3-carboxamide

To a suspension of 1-(3-bromophenyl)-1,4-dihydro[1,8]naphthyridin-4-one-3-carboxylic acid from Step 3 (26.3 g, 76 mmol) and triethylamine (23.2 g, 230 mmol) in tetrahydrofuran (100 mL) at 0° C. was added isobutyl chloroformate (18.85 g, 138 mmol). After stirring at 0° C. for 2 hours, isopropylamine (23 g, 390 mmol) was added and the mixture was allowed to warm up to room temperature and stirred overnight. The mixture was then partitioned between ethyl acetate and water, the organic phase was dried and evaporated to a solid which was stirred in ether at room temperature for 3 hours and filtered to afford the N-Isopropyl-1-(3-bromophenyl)-1,4-dihydro[1,8]naphthyridin-4-one-3-carboxamide as a white solid.

¹H NMR (Acetone-d₆) δ 1.25 (d, 6H), 4.17 (m, 1H), 7.59-7.63 (m, 2H), 7.70 (d, 1H), 7.80 (d, 1H), 7.94 (s, 1H), 8.73 (m, 1H), 8.78 (d, 1H), 8.85 (s, 1H), 9.61 (br, NH).

### Step 5: N-Isopropyl-1-[3-(3-acetylphenyl)phenyl]-1,4-dihydro [1,8]naphthyridin-4-one-3-carboxamide

A mixture of N-Isopropyl-1-(3-bromophenyl)-1,4-dihydro[1,8]naphthyridin-4-one-3-carboxamide from Step 4, 3-acetylphenylboronic acid (1.2 eq.), trans-dibromobis(triphenylphosphine)palladium (II) (0.05 eq.), toluene (6 ml/mmol), ethanol (2 mmol) and 2M aqueous sodium carbonate (8 eq.) was refluxed for 1 hour under a nitrogen atmosphere. The mixture was diluted with ethyl acetate and the organic phase was washed with water and brine, dried and evaporated. The crude product was chromatographed on silica gel eluting with a gradient of 20-40% ether in methylene chloride to afford the N-Isopropyl-1-[3-(3-acetylphenyl)phenyl]-1,4-dihydro[1,8]naphthyridin-4-one-3-carboxamide product as a solid.

¹H NMR (CDCl₃) δ 1.29 (d, 6H), 2.65 (s, 3H), 4.28 (m, 1H), 7.47 (m, 2H), 7.55 (t, 1H), 7.65 (m, 2H), 7.80 (m, 2H), 7.95 (dd, 1H), 8.19 (brs, 1H), 8.70 (dd, 1H), 8.81 (dd, 1H), 9.05 (s, 1H), 9.65 (br, NH).

### Example 2C

### N-(2,6-Dichloropyridin-4-yl)-1-[3-(3-acetylphenyl)phenyl]-1,4-dihydro[1,8]naphthyridin-4-one-3-carboxamide

### Step 1: Anion of 4-amino-3,5-dichloropyridine

A suspension of sodium hydride as 60% dispersion in oil (360 mg, 9 mmol) in tetrahydrofuran (15 mL) was cooled to 0° C. A solution of 4-amino-3,5-dichloropyridine (978 mg, 6 mmol) in tetrahydrofuran (15 mL) was added slowly. The resulting mixture was kept at 0° for 2.5 hours.

### Step 2: Acid chloride of 1-(3-bromophenyl)-1,4-dihydro[1,8]naphthyridin-4-one-3-carboxylic acid

A suspension of 1-(3-bromophenyl)-1,4-dihydro[1,8]naphthyridin-4-one-3-carboxylic acid from Step 3 of Example 1C (690 mg, 2 mmol) in tetrahydrofuran (12 mL) was cooled to 0° C., and oxalyl chloride (381 mg, 3 mmol) was added, followed by 2 drops of N,N-dimethylformamide. The resulting mixture was then stirred at room temperature for 1 hour then refluxed for 45 minutes and cooled to room temperature.

### Step 3: N-(2,6-Dichloropyridin-4-yl)-1-(3-bromophenyl)-1,4-dihydro[1,8]naphthyridin-4-one-3-carboxamide

The mixture from Step 2 above, as a brown suspension, was added via syringe to the cold suspension of Step 1. The resulting mixture was stirred at room temperature for 18 hours, quenched with aqueous saturated ammonium chloride solution and partitioned between ethyl acetate and water. The crude product from evaporation of the organic phase was triturated with ether (50 mL) and filtered, affording the N-(2,6-Dichloropyridin-4-yl)-1-(3-bromophenyl)-1,4-dihydro[1,8]naphthyridin-4-one-3-carboxamide as a beige solid.

¹H NMR (Acetone-d₆) δ 7.61-7.70 (m, 2H), 7.76 (d, 1H), 7.81 (d, 1H), 8.00 (s, 1H), 8.62 (s, 2H), 8.80 (br s, 1H), 8.86 (d, 1H), 8.99 (s, 1H), 12.1 (br, NH).

### Step 4: N-(2,6-Dichlorpyridin-4-yl)-1-[3-(3-acetylphenyl)phenyl]-1,4-dihydro[1,8]naphthyridin-4-one-3-carboxamide

Following the procedure of Step 5 of Example 1C, but substituting N-(2,6-dichloropyridin-4-yl)-1-(3-bromophenyl)-1,4-dihydro[1,8]naphthyridin-4-one-3-carboxamide from step 3 for N-isopropyl 1-(3-bromophenyl)-1,4-dihydro[1,8]naphthyridin-4-one-3-carboxamide, the N-(2,6-Dichloropyridin-4-yl)-1-[3-(3-acetylphenyl)phenyl]-1,4-dihydro[1,8]naphthyridin-4-one-3-carboxamide compound was obtained as a solid.

¹H NMR (CDCl₃) δ 2.65 (s, 3H), 7.47 (d, 1H), 7.50-7.60 (m, 2H), 7.70 (m, 2H), 7.82 (d, 2H), 7.98 (d, 1H), 8.20 (s, 1H), 8.55 (s, 2H) 8.75 (brs, 1H), 8.92 (dd, 1H), 9.14 (s, 1H), 12.08 (br, NH).

### Example 3C

N-Isopropyl-1-[3-(4-n-propylphenyl)phenyl]-1,4-dihydro[1,8]naphthyridin-4-one-3-carboxamide

Following the procedure of Step 5 of Example 1C, but substituting 4-n-propylphenylboronic acid for 3-acetylphenylboronic acid the title compound was obtained as a white solid.

¹H NMR (Acetone-4) δ 0.93 (t, 3H), 1.24 (d, 6H), 1.65 (m, 2H), 2.62 (t, 2H), 4.18 (m, 1H), 7.31 (d, 2H), 7.58-7.61 (m, 2H), 7.68-7.72 (m, 3H), 7.87 (d, 1H), 7.95 (s, 1H), 8.72 (m, 1H), 8.78 (dd, 1H), 8.92 (s, 1H), 9.66 (br, NH).

### Example 4C

### N-Isopropyl-1-[3-(4-acetylphenyl)phenyl]-1,4-dihydro[1,8]naphthyridin-4-one-3-carboxamide

Following the procedure of Step 5 of Example 1C, but substituting 4-acetylphenylboronic acid for 3-acetylphenylboronic acid the title compound was obtained as a solid.

¹H NMR (Acetone-d₆) δ 1.25 (d, 6H), 2.61 (s, 3H), 4.17 (m, 1H), 7.59 (m, 1H), 7.70 (d, 1H), 7.76 (t, 1H), 7.92 (d, 2H), 7.97 (d, 1H), 8.07-8.10 (m, 3H), 8.72 (brs, 1H), 8.78 (dd, 1H), 8.92 (s, 1H), 9.65 (br, NH).

### Example 5C

### N-Isopropyl-1-[3-(2-methylphenyl)phenyl]-1,4-dihydro[1,8]naphthyridin-4-one-3-carboxamide

Following the procedure of Step 5 of Example 1C, but substituting 2-methylphenylboronic acid for 3-acetylphenylboronic acid the title compound was obtained as a solid.

¹H NMR (Acetone-d₆) δ 1.24 (d, 6H), 2.35 (s, 3H), 4.17 (m, 1H), 7.27-7.34 (m, 4H), 7.56-7.60 (m, 2H), 7.65 (m, 2H), 7.70 (t, 1H), 8.74 (m, 1H). 8.78 (dd, 1H), 8.92 (s, 1H), 9.64 (br, NH).

### Example 6C

### N-Isopropyl-N-methyl-1-[3-(4-acetylphenyl)phenyl]-1,4-dihydro[1,8]naphthyridin-4-one-3-carboxamide

### Step 1: N-IsoproRyl-N-methyl-1-(3-bromophenyl)-1,4-dihydro[1,8]naphthyridin-4-one-3-carboxamide

Following the procedure of Example 1C, Step 4, but substituting N-isopropyl-N-methylamine for isopropylamine the N-Isopropyl-N-methyl-1-(3-bromophenyl)-1,4-dihydro[1,8]naphthyridin-4-one-3-carboxamide was obtained in as a yellow solid.

¹H NMR (Acetone-d₆) (Appears as two rotamers of the amide) δ 1.18 (m, 6H), 2.85 (s, 3H), 4.05 (m, 0.5H), 4.84 (m, 0.5H), 7.49-7.64 (m, 3H), 7.72 (d, 1H), 7.86 (s, 1H), 8.14 (s, 1H), 8.65 (d, 2H).

### Step 2: N-Isopropyl-N-methyl-1-[3-(4-acetylphenyl)phenyl]-1,4-dihydro[1,8]naphthyridin-4-one-3-carboxamide

Following the procedure of Step 5 of Example 1C, but substituting N-isopropyl-N-methyl-1-(3-bromophenyl)-1,4-dihydro[1,8]naphthyridin-4-one-3-carboxamide from step 1 for N-isopropyl 1-(3-bromophenyl)-1,4-dihydro[1,8]naphthyridin-4-one-3-carboxamide and 4-acetylphenylboronic acid for 3-acetylphenylboronic acid the N-Isopropyl-N-methyl-1-[3-(4-acetylphenyl)phenyl]-1,4-dihydro[1,8]naphthyridin-4-one-3-carboxamide compound was obtained as a white solid.

¹H NMR (CDCl₃) (Appears as two rotomers of the amide) δ 1.23 (m, 6H), 2.62 (s, 3H), 4.00 (m, 0.5H), 4.92 (m, 0.5H), 7.38-7.55 (m, 2H), 7.63-7.77 (m, 5H), 8.03 (d, 2H), 8.14 (s, 0.5H), 8.21 (s, 0.5H), 8.65 (m, 1H), 8.75-8.80 (m, 1H).

### Example 7C

### N-Isopropyl-1-[3-(pyridin-3-yl)phenyl]-1,4-dihydro[1,8]naphthyridin-4-one-3-carboxamide

Following the procedure of Step 5 of Example 1C, but substituting pyridine-3-boronic acid 1,3-propanediol cyclic ester for 3-acetylphenylboronic acid and [1,1′-bis (diphenylphosphino)ferrocene]dichloropalladium(II) for trans-dibromobis(triphenylphosphine)palladium (II) the title compound was obtained as a beige solid.

¹H NMR (Acetone-d₆) δ 1.24 (d, 6H), 4.17 (m, 1H), 7.48 (m, 1H), 7.60 (m, 1H), 7.71 (dd, 1H), 7.78 (t, 1H), 7.95 (dd, 1H), 8.05 (brs, 1H), 8.15 (m, 1H), 8.60 (m, 1H), 8.72 (m, 1H), 8.78 (dd, 1H), 8.92 (s, 1H), 8.99 (brs, 1H), 9.65 (br, NH).

### Example 8C

### N-Isopropyl-1-[3-(indol-5-yl)phenyl]-1,4-dihydro[1,8]naphthyridin-4-one-3-carboxamide

Following the procedure of Step 5 of Example 1C, but substituting 5-indolylboronic acid for 3-acetylphenylboronic acid the title compound was obtained as an off-white solid.

¹H NMR ((DMSO-d₆) δ 1.20 (d, 6H), 4.10 (m, 1H), 6.47 (s, 1H), 7.38 (brs, 1H), 7.46-7.52 (m, 3H), 7.59-7.66 (m, 2H), 7.87-7.93 (m, 3H), 8.72-8.81 (m, 3H), 9.67 (br, NH), 11.2 (br, NH).

### Example 9C

### N-tert-Butyl-1-[3-(4-acetylphenyl)phenyl]-1,4-dihydro[1,8]naphthyridin-4-one-3-carboxamide

### Step 1: N-tert-Butyl-1-(3-bromophenyl)-1,4-dihydro[1,8]naphthyridin-4-one-3-carboxamide

Following the procedure of Example 1C, Step 4, but substituting tert-butylamine for isopropylamine the N-tert-Butyl-1-(3-bromophenyl)-1,4-dihydro[1,8]naphthyridin-4-one-3-carboxamide was obtained as a yellow solid.

¹H NMR (Acetone-d₆) δ 1.44 (s, 9H), 7.58-7.62 (m, 2H), 7.70 (dd, 1H), 7.78 (dd, 1H), 7.93 (br s, 1H), 8.72 (m, 1H), 8.77 (dd, 1H), 8.81 (s, 1H), 9.73 (br, NH).

### Step 2: N-tert-Butyl-1-[3-(4-acetylphenyl)phenyl]-1,4-dihydro[1,8]naphthyridin-4-one-3-carboxamide

Following the procedure of Step 5 of Example 1C, but substituting N-tert butyl-1-(3-bromophenyl)-1,4-dihydro[1,8]naphthyridin-4-one-3-carboxamide from step 1 for N-isopropyl 1-(3-bromophenyl)-1,4-dihydro[1,8]naphthyridin-4-one-3-carboxamide and 4-acetylphenylboronic acid for 3-acetylphenylboronic acid the N-tert-Butyl-1-[3-(4-acetylphenyl)phenyl]-1,4-dihydro[1,8]naphthyridin-4-one-3-carboxamide compound was obtained in 93% yield as a white solid.

¹H NMR (Acetone-d₆) δ 1.45 (s, 9H), 2.61 (s, 3H), 7.59 (m, 1H), 7.69-7.72 (m, 1H), 7.77 (t, 1H), 7.92-7.99 (m, 3H), 8.07-8.11 (m, 3H), 8.72 (m, 1H), 8.78 (dd, 1H), 8.91 (s, 1H), 9.79 (br, NH).

### Example 10C

### N-(2,6-Dichloropyridin-4-yl)-1-[3-(pyridin-3-yl)phenyl]-1,4-dihydro[1,8]naphthyridin-4-one-3-carboxamide

Following the procedure of Step 4 of Example 2C but substituting [1,1′-bis (diphenylphosphino)ferrocene]dichloropalladium(II) for trans-dibromobis(triphenylphosphine)palladium (II) and pyridine-3-boronic acid 1,3-propanediol cyclic ester for 3-acetylphenylboronic acid the title compound was obtained as a glassy solid.

H NMR (Acetone-d₆) δ 7.48 (m, 1H), 7.68 (m, 1H), 7.77-7.82 (m, 2H), 7.98 (m, 1H), 8.12-8.17 (m, 2H), 8.61 (m, 1H), 8.62 (s, 2H), 8.80 (m, 1H), 8.88 (dd, 1H), 8.99 (brs, 1H), 9.06 (s, 1H), 12.2 (br, NH).

### Example 11C

### N-Isopropyl-1-{3-[4-(4-tertbutyloxycarbonylpiperazin-1-yl)phenyl]-phenyl}-1,4-dihydro[1,8]naphthyridin-4-one-3-carboxamide

### Step 1: 4-tert-Butyloxycarbonyl-1-(3-bromophenyl)piperazine

To a suspension of 1-(4-bromophenyl)piperazine hydrochloride (103.15 g, 371.59 mmol) in acetonitrile (1.5 L) at 0° C. under a nitrogen atmosphere was added a catalytic amount of 4-dimethylaminopyridine (4.54 g, 37.159 mmol) followed by triethylamine (155 mL, 1114.77 mmol) and di-tert-butyl dicarbonate (121.65 g, 557.385 mmol, dissolved in a minimum amount of acetonitrile) and the resulting reaction mixture was warmed to room temperature and stirred for 5.5 hours. The reaction mixture was filtered, ethyl acetate was added and the organic phase was washed with 10% aqueous citric acid, water (2×) and brine, then dried and evaporated to afford the crude 4-tert-Butyloxycarbonyl-1-(3-bromophenyl)piperazine product which was used as such in the next step.

### Step 2: 3-(4-tert-Butyloxycarbonylpiperazin-1-yl)phenylboronic acid

To the 4-tert-Butyloxycarbonyl-1-(3-bromophenyl)piperazine from Step 1 (118.30 g, 346.9 mmol) in tetrahydrofuran/toluene (1/1, 1.5 L) at −78° C. under nitrogen was added n-butyllithium (2.5M, 160 mL, 398.9 mmol) dropwise and the resulting reaction mixture was stirred at −78° C. for 20 minutes. Triisopropyl borate (96.1 mL, 416.3 mmol) was added dropwise and the reaction was warmed to 0° C. and stirred for 2 hours. Aqueous saturated ammonium chloride (400 mL), water (100 mL) and 1 equivalent of H₃PO₄ (20 mL) were added and the mixture was stirred for 15 minutes and then concentrated to a volume of approximately 200 mL (at which stage the mixture became bluish and a precipitate formed). The mixture was slowly diluted with heptane (800 mL) and the resulting suspension was stirred overnight. The suspension was filtered, the solid was washed with heptane and dried to afford the title boronic acid.

### Step 3: N-Isopropyl-1-{3-[4-(4-tertbutyloxycarbonylpiperazin-1-yl)phenyl]-phenyl}-1,4-dihydro[1,8]naphthyridin-4-one-3-carboxamide

Following the procedure of Step 5 of Example 1C but substituting [1,1′-bis (diphenylphosphino)ferrocene]dichloropalladium(II) for trans-dibromobis(triphenylphosphine)palladium (II) and the boronic acid from Step 2 above for 3-acetylphenylboronic acid the 4-tert-Butyloxycarbonyl-1-(3-bromophenyl)piperazine compound was obtained as a solid.

¹H NMR (CDCl₃) δ 1.30 (d, 6H), 1.49 (s, 9H), 3.18 (m, 4H), 3.58 (m, 4H), 4.29 (m, 1H), 6.98 (d, 2H), 7.32 (d, 1H), 7.45 (m, 1H), 7.53 (d, 2H), 7.55-7.62 (m, 2H), 7.72 (d, 1H), 8.70 (m, 1H), 8.82 (d, 1H), 9.07 (s, 1H), 9.68 (br, NH).

### Example 12C

### N-Isopropyl-1-[3-(quinolin-3-yl)phenyl]-1,4-dihydro[1,8]naphthyridin-4-one-carboxamide

Following the procedure of Step 5 of Example 1C, but substituting 3-quinolineboronic acid for 3-acetylphenylboronic acid the title compound was obtained as a solid.

¹H NMR (CDCl₃) δ 1.29 (d, 6H), 4.29 (m, 1H), 7.49 (m, 2H), 7.61 (t, 1H), 7.70-7.78 (m, 3H), 7.86-7.92 (m, 2H), 8.14 (d, 1H), 8.36 (s, 1H), 8.71 (m, 1H), 8.84 (dd, 1H), 9.10 (s, 1H), 9.19 (s, 1H), 9.67 (br, NH).

### Example 13C

### N-Isopropyl-1-[3-(pyrimidin-5-yl)phenyl]-1,4-dihydro[1,8]naphthyridin-4-one-carboxamide

Following the procedure of Step 5 of Example 1C, but substituting 5-pyrimidineboronic acid for 3-acetylboronic acid the title compound was obtained as a solid.

¹H NMR (CDCl₃) δ 1.28 (d, 6H), 4.27 (m, 1H), 7.48 (dd, 1H), 7.52 (m, 1H), 7.65 (s, 1H), 7.74 (m, 2H), 8.68 (m, 1H), 8.72 (d, 1H), 8.98 (s, 2H) 9.03 (s, 1H), 9.22 (s, 1H), 9.62 (br, NH).

### Example 14C

### N-Cyclopropyl-1-[3-(pyridin-3-yl)phenyl]-1,4-dihydro[1,8]naphthyridin-4-one-3-carboxamide

### Step 1: N-Cyclopropyl-1-(3-bromophenyl)-1,4-dihydro[1,8]naphthyridin-4-one-3-carboxamide

Following the procedure of Example 1C, Step 4, but substituting cyclopropylamine for isopropylamine the N-Cyclopropyl-1-(3-bromophenyl)-1,4-dihydro[1,8]naphthyridin-4-one-3-carboxamide was obtained as a fluffy white solid.

¹H NMR (Acetone-d₆) δ 0.59 (m, 2H), 0.80 (m, 2 h), 2.96 (m, 1H), 7.59-7.68 (m, 2H), 7.72 (dd, 1H), 7.82 (dd, 1H), 7.97 (s, 1H), 8.72-8.81 (m, 2H), 8.89 (s, 1H), 9.70 (br, NH).

### Step 2: N-Cyclopropyl-1-[3-(pyridin-3-yl)phenyl]-1,4-dihydro[1,8]naphthyridin-4-one-3-carboxamide

Following the procedure of Example 7C but substituting N-cyclopropyl-1-(3-bromophenyl)-1,4-dihydro[1,8]naphthyridin-4-one-3-carboxamide from step 1 for N-isopropyl-1-(3-bromophenyl)-1,4-dihydro[1,8]naphthyridin-4-one-3-carboxamide, the N-Cyclopropyl-1-[3-(pyridin-3-yl)phenyl]-1,4-dihydro[1,8]naphthyridin-4-one-3-carboxamide compound was obtained as a cream-coloured solid.

¹H NMR (DMSO-d⁶) δ 0.57 (m, 2H), 0.78 (m, 2H), 2.91 (m, 1H), 7.52 (m, 1H), 7.63-7.69 (m, 2H), 7.74 (t, 1H), 7.97 (d, 1H), 8.07 (brs, 1H), 8.17 (d, 1H), 8.61 (m, 1H), 8.73 (dd, 1H), 8.79 (m, 1H), 8.85 (s, 1H), 8.99 (brs, 1H), 9.74 (br, NH).

### Example 15C

### N-Isopropyl-1-[3-(5-methylthiopyridin-3-yl)phenyl]-1,4-dihydro[1,8]naphthyridin-4-one-3-carboxamide

Following the procedure of Step 5 of Example 1C but substituting 5-methylthiopyridine-3-boronic acid for 3-acetylphenylboronic acid and [1,1′-bis (diphenylphosphino)ferrocene]dichloropalladium(II) for trans-dibromobis(triphenylphosphine)palladium (II) the title compound was obtained as a solid.

¹H NMR (CDCl₃) δ 1.33 (d, 6H) 2.60 (s, 3H), 4.33 (m, 1H), 7.48-7.54 (m, 2H), 7.66 (m, 1H), 7.73 (t, 1H), 7.78-7.81 (m, 2H), 8.55 (s, 1H), 8.66 (s, 1H), 8.74 (m, 1H), 8.87 (d, 1H), 9.09 (s, 1H), 9.69 (br, NH).

### Example 16C

### N-Cyclopropyl-1-[3-(4-hydroxymethylphenyl)phenyl]-1,4-dihydro[1,8]naphthyridin-4-one-3-carboxamide

Following the procedure of Step 2 of Example 14C but substituting 4-hydroxymethylphenyl boronic acid for pyridine-3-boronic acid 1,3-propanediol cyclic ester the title compound was obtained as a solid.

¹H NMR (CDCl₃), 0.71 (m, 2H), 0.89 (m, 2H), 1.88 (t, 1H), 3.03 (m, 1H), 4.78 (d, 2H), 7.43 (d, 1H), 7.46-7.52 (m, 3H), 7.61-7.69 (m, 4H), 7.80 (d, 1H), 8.73 (m, 1H), 8.83 (dd, 1H), 9.10 (s, 1H), 9.82 (br, NH).

### Example 17C

### N-Cyclopropyl-1-[3-(pyridin-4-yl)phenyl]-1,4-dihydro[1,8]naphthyridin-4-one-3-carboxamide

Following the procedure of Step 5 of Example 1C but substituting N-cyclopropyl-1-(3-bromophenyl)-1,4-dihydro[1,8]naphthyridin-4-one-3-carboxamide for N-isopropyl-1-(3-bromophenyl)-1,4-dihydro[1,8]naphthyridin-4-one-3-carboxamide and 4-pyridineboronic acid for 3-acetylphenylboronic acid the title compound was obtained as a white solid.

¹H NMR ((DMSO-d⁶) δ 0.57 (m, 2H), 0.77 (m, 2H), 2.90 (m, 1H), 7.64 (m, 1H), 7.72-7.89 (m, 4H), 8.03 (d, 1H), 8.13 (s, 1H), 8.66-8.78 (m, 4H), 8.84 (s, 1H), 9.72 (br, NH).

### Example 18C

### N-Cyclopropyl-1-[3-(4-ethylthiophenyl)phenyl]-1,4-dihydro[1,8]naphthyridin-4-one-3-carboxamide

Following the procedure of Step 2 of Example 14C but substituting 4-ethylthiobenzeneboronic acid for pyridine-3-boronic acid 1,3-propanediol cyclic ester the title compound was obtained as a solid.

¹H NMR (CDCl₃) δ 0.72 (m, 2H), 0.90 (m, 2H), 1.48 (t, 3H), 3.03 (m, 3H), 7.42 (d, 3H), 7.50 (m, 1H), 7.57 (d, 2H), 7.64 (s, 1H), 7.68 (t, 1H), 7.78 (d, 1H), 8.75 (m, 1H), 8.85 (d, 1H), 9.10 (s, 1H), 9.83 (br, NH).

### Example 19C

### N-Cyclopropyl-1-[3-(3-thienyl)phenyl]-1,4-dihydro[1,8]naphtherdin-4-one-3-carboxamide

Following the procedure of Step 2 of Example 14C but substituting 3-thiopheneboronic acid for pyridine-3-boronic acid 1,3-propanediol cyclic ester the title compound was obtained as a white solid.

¹H NMR (Acetone-d₆) δ 0.60 (m, 2H), 0.79 (m, 2H), 2.96 (m, 1H), 7.57-7.72 (m, 5H), 7.92-7.98 (m, 2H), 8.05 (s, 1H), 8.74 (s, 1H), 8.78 (d, 1H), 8.93 (s, 1H), 9.74 (br, NH).

### Example 20C

### N-Cyclopropyl-1-[3-(4-sulfamoylphenyl)phenyl]-1,4-dihydro[1,8]naphthyridin-4-one-3-carboxamide

### Step 1: 4-Sulfamoylbenzeneboronic acid pinacol ester

A mixture of 4-bromobenzenesulfonamide, diboron pinacol ester (1.1 eq), potassium acetate (3.5 eq) and 1,1′-bis (diphenylphosphino)ferrocene]dichloropalladium(II) (0.05 eq) im N,N-dimethylformamide (4 ml/mmol) was heated at 85° C. for 18 hours. After quenching with saturated aqueous ammonium chloride solution the mixture was partitioned between ethyl acetate and water and the product from the organic phase was chromatographed on silica gel eluting with a 1:1 mixture of ethyl acetate and hexane to afford the 4-Sulfamoylbenzeneboronic acid pinacol ester as a solid.

### Step 2: N-Cyclopropyl-1-[3-(4-sulfamoylphenyl)phenyl]-1,4-dihydro[1,8]naphthyridin-4-one-3-carboxamide

A mixture of N-cyclopropyl-1-(3-bromophenyl)-1,4-dihydro[1,8]naphthyridin-4-one-3-carboxamide, boronate from Step 1 (1.2 eq), palladium acetate (0.1 eq), triphenylphosphine (0.35 eq) and 2M aqueous sodium carbonate (3.5 eq) in n-propanol (10 ml/mmol) was stirred at 85° C. for 1 hour. After cooling, the mixture was quenched with saturated aqueous ammonium chloride solution and partitioned between ethyl acetate and water, and the product from the organic phase was chromatographed on silica gel eluting with a 1:5:4 mixture of ethanol, ethyl acetate and methylene chloride to afford the N-Cyclopropyl-1-[3-(4-sulfamoylphenyl)phenyl)-1,4-dihydro[1,8]naphthyridin-4-one-3-carboxamide compound as a solid.

¹H NMR (Acetone-d₆) δ 0.62 (m, 2H), 0.82 (m, 2H), 2.98 (m, 1H), 6.66 (br, NH₂), 7.64 (m, 1H), 7.74 (m, 1H), 7.80 (t, 1H), 7.97-8.05 (m, 5H), 8.10 (m, 1H), 8.76 (m, 1H), 8.81 (dd, 1H), 8.97 (s, 1H), 9.77 (br, NH).

### Example 21C

### N-Isopropyl-1-[3-(3-ethoxyphenyl)phenyl]-1,4-dihydror[1,8]naphthyridin-4-one-3-carboxamide

### Step 1: Ethyl 1-[3-(3-ethoxyphenyl)phenyl]-1,4-dihydro[1,8]naphthyridin-4-one-3-carboxylate

Following the procedure of Step 5 of Example 1C, but substituting ethyl 1-(3-bromophenyl)-1,4-dihydro[1,8]naphthyridin-4-one-3-carboxylate from Step 2 of Example 1 for N-isopropyl-1-(3-bromophenyl)-1,4-dihydro[1,8]naphthyridin-4-one-3-carboxamide, and 3-ethoxybenzeneboronic acid for 3-acetylbenzeneboronic acid, the Ethyl 1-[3-(3-ethoxyphenyl)phenyl]-1,4-dihydro[1,8]naphthyridin-4-one-3-carboxylate compound was obtained as a solid.

### Step 2: 1-[3-(3-Ethoxyphenyl)phenyl]-1,4-dihydro[1,8]naphthyridin-4-one-3-carboxylic acid

Following the procedure of Step 3 of Example 1C but substituting ethyl 1-[3-(3-ethoxyphenyl)phenyl]-1,4-dihydro[1,8]naphthyridin-4-one-3-carboxylate from step 1 for ethyl 1-(3-bromophenyl)-1,4-dihydro[1,8]naphthyridin-4-one-3-carboxylate the 1-[3-(3-Ethoxyphenyl)phenyl]-1,4-dihydro[1,8]naphthyridin-4-one-3-carboxylic acid compound was obtained and used without purification in the next step.

### Step 3: N-Isopropyl-1-[3-(3-ethoxyphenyl)phenyl]-1,4-dihydro[1,8]naphthyridin-4-one-3-carboxamide

A mixture of 1-[3-(3-ethoxyphenyl)phenyl]-1,4-dihydro[1,8]naphthyridin-4-one-3-carboxylic acid from Step 2 and thionyl chloride (4 eq) in tetrahydrofuran (10 ml/mmol) was refluxed for 45 minutes, then evaporated. The residue was dissolved in the same volume of tetrahydrofuran, isopropylamine (Seq) was added and the mixture was stirred at room temperature for 18 hours. After quenching with saturated aqueous ammonium chloride solution, the resulting mixture was partitioned between ethyl acetate and water, and the product from the organic phase was chromatographed on silica gel eluting with 10% ether in methylene chloride to afford the N-Isopropyl-1-[3-(3-ethoxyphenyl)phenyl]-1,4-dihydro[1,8]naphthyridin-4-one-3-carboxamide compound as a solid.

¹H NMR (CDCl₃) δ 1.29 (d, 6H), 1.42 (t, 3H), 4.08 (q, 2H), 4.28 (m, 1H), 6.91 (d, 1H), 7.12 (s, 1H), 7.18 (d, 1H), 7.34 (t, 1H), 7.40 (d, 1H), 7.46 (m, 1H), 7.60-7.65 (m, 2H), 7.75 (d, 1H), 8.71 (brs, 1H), 8.82 (dd, 1H), 9.08 (s, 1H), 9,70 (br, NH).

### Example 22C

### N-Isopropyl-1-[3-(4-methylthiophenyl)phenyl]-1,4-dihydro[1,8]naphthyridin-4-one-3-carboxamide

### Step 1: Ethyl 1-[3-(4-methylthiophenyl)phenyl]-1,4-dihydro[1,8]naphthyridin-4-one-3-carboxylate

Following the procedure of Step 5 of Example 1C, but substituting ethyl 1-(3-bromophenyl)-1,4-dihydro[1,8]naphthyridin-4-one-3-carboxylate from step 2 of example 1 for N-isopropyl-1-(3-bromophenyl)-1,4-dihydro[1,8]naphthyridin-4-one-3-carboxamide, and 4-methylthiobenzeneboronic acid for 3-acetylbenzeneboronic acid, the Ethyl 1-[3-(4-methylthiophenyl)phenyl]-1,4-dihydro[1,8]naphthyridin-4-one-3-carboxylate compound was obtained as a solid.

### Step 2: 1-[3-(4-methylthiophenyl)phenyl]-1,4-dihydro[1,8]naphthyridin-4-one-3-carboxylic acid

Following the procedure of Step 3 of Example 1C but substituting ethyl 1-[3-(4-methylthiophenyl)phenyl]-1,4-dihydro[1,8]naphthyridin-4-one-3-carboxylate from Step 1 for ethyl 1-(3-bromophenyl)-1,4-dihydro[1,8]naphthyridin-4-one-3-carboxylate the 1-[3-(4-methylthiophenyl)phenyl]-1,4-dihydro[1,8]naphthyridin-4-one-3-carboxylic acid compound was obtained as a solid.

### Step 3: N-Isopropyl-1-[3-(4-methylthiophenyl)phenyl]-1,4-dihydro[1,8]naphthyridin-4-one-3-carboxamide

Following the procedure of Step 3 of Example 21C but substituting 1-[3-(4-methylthiophenyl)phenyl]-1,4-dihydro[1,8]naphthyridin-4-one-3-carboxylic acid for 1-[3-(3-ethoxyphenyl)phenyl]-1,4-dihydro[1,8]naphthyridin-4-one-3-carboxylic acid the N-Isopropyl-1-[3-(4-methylthiophenyl)phenyl]-1,4-dihydro[1,8]naphthyridin-4-one-3-carboxamide compound was obtained as a white solid.

¹H NMR (Acetone-d₆) δ 1.24 (d, 6H), 2.52 (s, 3H), 4.18 (m, 1H), 7.37 (d, 2H), 7.58-7.62 (m, 2H), 7.69-7.73 (m, 3H), 7.87 (d, 1H), 7.96 (s, 1H), 8.72 (m, 1H), 8.78 (dd, 1H), 8.91 (s, 1H), 9.65 (br, NH).

### Example 23C

### N-Isopropyl-1-[3-(3-acetyl-4-hydroxyphenyl)phenyl]-1,4-dihydro[1,8]naphthyridin-4-one-3-carboxamide

A mixture of 5′-bromo-2′-hydroxyacetophenone, diboron pinacol ester (1.25 eq), potassium acetate (3 eq) and [1,1′-bis (diphenylphosphino)ferrocene]dichloropalladium(II) (0.05 eq) in N,N-dimethylformamide (10 ml/mmol) was stirred at 80° C. for 3 hours and cooled down. A solution of N-Isopropyl-1-(3-bromophenyl)-1,4-dihydro[1,8]naphthyridin-4-one-3-carboxamide from Example 1, Step 4 (0.75 eq) in N,N-dimethylformamide (7 ml/mmol), [1,1′-bis(diphenylphosphino)ferrocene]dichloropalladium(II) (0.05 eq) and 2M aqueous sodium carbonate (8.5 eq) were added and the resulting mixture was stirred at 80° C. for 2.5 hours. The cooled mixture was partitioned between ethyl acetate and water and the product from the organic phase was chromatographed on silica gel eluting with 60% ethyl acetate in hexane to afford the title compound as a light yellow solid.

¹H NMR (Acetone-d₆) δ 1.24 (d, 6H), 2.75 (s, 3H), 4.19 (m, 1H), 7.06 (d, 1H), 7.59-7.63 (m, 2H), 7.72 (t, 1H), 7.92 (d, 1H), 7.97 (d, 1H), 8.02 (s, 1H), 8.33 (s, 1H), 8.73 (m, 1H), 8.78 (dd, 1H), 8.90 (s, 1H), 9.65 (br, NH).

### EXAMPLE 24C

### N-Isopropyl-1-[3-(5-carboethoxypyridin-3-yl)phenyl]-1,4-dihydro[1,8]naphthyridin-4-one-3-carboxamide

Following the procedure of Example 23C but substituting N-isopropyl-1-(3-bromophenyl)-1,4-dihydro[1,8]naphthyridin-4-one-3-carboxamide for 5′-bromo-2′-hydroxyacetophenone and ethyl 5-bromonicotinate for N-isopropyl-1-(3-bromophenyl)-1,4-dihydro[1,8]naphthyridin-4-one-3-carboxamide the title compound was obtained as a beige solid.

¹H NMR (CDCl₃) δ 1.29 (d, 6H), 1.40 (t, 3H), 4.28 (m, 1H), 4.42 (q, 2H), 7.45-7.51 (m, 21), 7.68 (s, 1H), 7.71 (t, 1H), 7.80 (d, 1H), 8.49 (s, 1H), 8.59 (m, 1H), 8.82 (d, 1H), 9.03 (s, 1H), 9.07 (s, 1H), 9.23 (s, 1H), 9.64 (br, NH).

### Example 25C

### N-Isopropyl-1-{3-[5-(1-hydroxy-1-methylethyl)pyridin-3-yl]phenyl}-1,4-dihydro[1,8]naphthyridin-4-one-3-carboxamide

### Step 1: 3-Bromo-5-(1-hydroxy-1-methylethyl)pyridine

To a solution of ethyl 5-bromonicotinate (1.02 g, 4.4 mmol) in diethyl ether (15 ml) at −30° C. was added a 3M solution of methyl magnesium bromide (4 ml, 12 mmol)in ether. The resulting slurry was then refluxed for 2 hours then cooled and quenched with an excess of 0.5M aqueous monobasic sodium phosphate and partitioned between ether and water. The product from the organic phase was chromatographed on silica gel eluting with a 2:1:2 mixture of ether, pentane and ammonia-saturated methylene chloride to afford the 3-Bromo-5-(1-hydroxy-1-methylethyl)pyridine compound as a yellow oil.

### Step 2: N-Isopropyl-1-{3-[5-(1-hydroxy-1-methylethyl)pyridin-3-yl]phenyl}-1,4-dihydro[1,8]naphthyridin-4-one-3-carboxamide

Following the procedure of Example 24C, but substituting the 3-bromo-5-(1-hydroxy-1-methylethyl)pyridine from Step 1 for ethyl 5-bromonicotinate, the title compound was obtained as a yellow foam.

¹H NMR (CDCl₃) δ 1.28 (d, 6H), 1.62 (s, 6H), 2.52 (brs, 1H), 4.25 (m, 1H), 7.41-7.48 (m, 2H), 7.60-7.68 (m, 2H), 7.75 (d, 1H), 8.05 (s, 1H), 8.67-8.71 (m, 3H), 8.80 (dd, 1H), 9.03 (s, 1H), 9.66 (br, NH).

### EXAMPLE 26C

### N-Isopropyl-1-{3-[6-(2-methylpropyl)pyridin-3-yl]phenyl}-1,4-dihydro[1,8]naphthyridin-4-one-3-carboxamide

### Step 1: 5-Bromo-2-(2-methylpropyl)pyridine

To a solution of 2,5-dibromopyridine (4.5 g, 19 mmol) in tetrahydrofuran (50 ml) was added [1,1′-bis (diphenylphosphino)ferrocene]dichloronickel (1) (103 mg, 0.19 mmol) and the resulting mixture was cooled to −10° C. A 2M solution of isobutylmagnesium bromide in ether (12.4 ml, 24.7 mmol) was added slowly and the mixture was stirred at −10 to 10° C. for 3.5 hours. After quenching with saturated aqueous ammonium chloride solution, the mixture was partitioned between ether and water and the product from the organic phase was chromatographed on silica gel eluting with 10% ether in pentane to afford the 5-Bromo-2-(2-methylpropyl)pyridine compound as a volatile oil.

### Step 2: N-Isopropyl-1-{3-[6-(2-methylpropyl)pyridin-3-yl]phenyl}-1,4-dihydro[1,8]naphthyridin-4-one-3-carboxamide

Following the procedure of Example 24C but substituting 5-bromo-2-(2-methylpropyl)pyridine from Step 1 for ethyl 5-bromonicotinate the N-Isopropyl-1-{3-[6-(2-methylpropyl)pyridin-3-yl]phenyl}-1,4-dihydro[1,8]naphthyridin-4-one-3-carboxamide compound was obtained as a white solid.

¹H NMR (CDCl₃) δ 0.92 (d, 6H), 1.28 (d, 6H), 2.10 (m, 1H), 2.69 (d, 2H), 4.28 (m, 1H), 7.19 (d, 1H), 7.40-7.47 (m, 2H), 7.60 (s, 1H), 7.64 (t, 1H), 7.73 (d, 1H), 7.79 (dd, 1H), 8.68 (m, 1H), 8.77-8.83 (m, 2H), 9.05 (s, 1H), 9.66 (br, NH).

### Example 27C

### N-Isopropyl-1-[3-(5-acetylpyridin-3-yl)phenyl]-1,4-dihydro(1.8]naphthyridin-4-one-3-carboxamide

### Step 1: 3-Acetyl-5-bromopyridine

To a solution of ethyl 5-bromonicotinate (3.9 g, 16.9 mmol) in ether (50 ml) at 0° C. was added a 3M solution of methylmagnesium bromide (16.9 ml, 50.8 mmol). The resulting thick slurry was warmed slowly to room temperature and after 1.5 hours it was poured slowly into an excess of 1M aqueous monobasic sodium phosphate. The mixture was partitioned between ether and water and the product from the organic phase was chromatographed on silica gel, eluting with a 1:1:2 mixture of ether, pentane and ammonia-saturated methylene chloride to afford the 3-acetyl-5-bromopyridine compound. This preparation also afforded 3-bromo-5-(1-hydroxy-1-methylethyl)pyridine described in Example 25C.

### Step 2: N-Isopropyl-1-[3-(5-acetylpyridin-3-yl)phenyl]-1,4-dihydro[1,8]naphthyridin-4-one-3-carboxamide

Following the procedure of Example 23C but substituting 3-acetyl-5-bromopyridine from Step 1 for ethyl 5-bromonicotinate the N-isopropyl-1-[3-(5-acetylpyridin-3-yl)phenyl]-1,4-dihydro[1,8]naphthyridin-4-one-3-carboxamide compound was obtained as a white solid.

¹H NMR (CDCl₃) δ 1.29 (d, 6H), 2.69 (s, 3H), 4.28 (m, 1H), 7.48 (dd, 1H), 7.51 (d, 1H), 7.69 (s, 1H), 7.72 (t, 1H), 7.80 (d, 1H), 8.42 (s, 1H), 8.69 (m, 1H), 8.82 (d, 1H), 9.05 (s, 2H) 9.17 (s, 1H), 9.63 (br, NH).

### Example 28C

### N-Isopropyl-1-[3-(6-methylpyridin-3-yl)phenyl]-1,4-dihydro[1,8]naphthyridin-4-one-3-carboxamide

### Step 1: 5-Bromo-2-methylpyridine

Following the procedure of Step 1 of Example 26C but substituting methylmagnesium chloride for isobutylmagnesium bromide the 5-bromo-2-methylpyridine compound was obtained as a solid.

### Step 2: N-Isopropyl-1-[3-(6-methylpyridin-3-yl)phenyl]-1,4-dihydro[1,8]naphthyridin-4-one-3-carboxamide

Following the procedure of Example 24C but substituting 5-bromo-2-methylpyridine from Step 1 for ethyl 5-bromonicotinate the N-Isopropyl-1-[3-(6-methylpyridin-3-yl)phenyl]-1,4-dihydro[1,8]naphthyridin-4-one-3-carboxamide compound was obtained as a solid.

¹H NMR (CDCl₃) δ 1.32 (d, 6H), 2.63 (m, 3H), 4.30 (m, 1H), 7.25 (d, 1H), 7.45-7.51 (m, 2H), 7.63 (s, 1H), 7.69 (t, 1H), 7.77 (d, 1H), 7.82 (dd, 1H), 8.72 (m, 1H), 8.78 (s, 1H), 8.85 (d, 1H), 9.08 (s, 1H), 9.68 (br, NH).

### Example 29C

### N-Cyclopropyl-1-[3-(1-oxidopyrimidin-5-yl) phenyl]-1,4-dihydro[1.8]naphthyridin-4-one-3-carboxamide

### Step 1: 5-Bromo-1-oxidopyrimidine

To 5-bromopyrimidine (2.05 g, 12.9 mmol) in methylene chloride (25 ml) was added m-chloroperoxybenzoic acid (ca 70% pure, 3.17 g, 12.9 mmol) and the resulting mixture was stirred at room temperature for 5 days. Calcium hydroxide (1 g) was added and after 10 minutes the mixture was filtered through celite. The product from evaporation of the filtrate was chromatographed on silica gel eluting with ethyl acetate to afford the 5-bromo-1-oxidopyrimidine compound as a white solid.

### Step 2: N-Cyclopropyl-1-[3-(1-oxidopyrimidinyl-5-yl)phenyl]-1,4-dihydro[1,8]naphthyridin-4-one-3-carboxamide

Following the procedure of Example 24C but substituting 5-bromo-1-oxidopyrimidine from Step 1 for ethyl 5-bromonicotinate and N-cyclopropyl-1-(3-bromophenyl)-1,4-dihydro[1,8]naphthyridin-4-one-3-carboxamide for N-isopropyl-1-(3-bromophenyl)-1,4-dihydro[1,8]naphthyridin-4-one-3-carboxamide, the N-Cyclopropyl-1-[3-(1-oxidopyrimidinyl-5-yl)phenyl]-1,4-dihydro[1,8]naphthyridin-4-one-3-carboxamide compound was obtained as a white solid.

¹H NMR (CDCl₃) δ 0.66 (m, 2H), 0.84 (m, 2H), 2.97 (m, 1H), 7.48 (m, 1H), 7.58 (d, 1H), 7.65 (s, 1H), 7.71 (d, 1H), 7.77 (t, 1H), 8.46 (s, 1H), 8.60 (s, 1H), 8.68 (brs, 1H), 8.81 (dd, 1H), 8.98 (s, 1H), 9.02 (s, 1H), 9.72 (br, NH).

### Example 30C

### 1-{3-[6-(1-Hydroxy-1-methylethyl)-1-oxidopyridin-3-yl]phenyl}-1,4-dihydro[1,8]naphthyridin-4-one-3-carboxamide

### Step 1: 5-Bromo-2-(1-hydroxy-1-methylethyl)pyridine

To a suspension of 2,5-dibromopyridine in toluene (12 ml/mmol) cooled to −78° C. was added n-butyllithium 2.5M in hexanes (1.05 eq) and the resulting mixture was stirred in the cold for 2.5 hours. Acetone (2 eq) was added and stirring was continued for 1.5 h. After quenching with saturated aqueous ammonium chloride solution, the mixture was warmed to room temperature and partitioned between ethyl acetate and water. The product from the organic phase was chromatographed on silica gel eluting with 20% ethyl acetate in hexane to afford the 5-Bromo-2-(1-hydroxy-1-methylethyl)pyridine compound as a syrup.

### Step 2: 5-Bromo-2-(1-hydroxy-1-methylethyl)pyridine N-oxide

To a solution of 5-bromo-2-(1-hydroxy-1-methylethyl)pyridine from Step 1 in methylene chloride (5 ml/mmol) at room temperature was added m-chloroperoxybenzoic acid 70% (1.1 eq) and the resulting mixture was stirred at room temperature for 18 hours. An excess of calcium hydroxide was added and after 5 minutes the mixture was filtered through a bed of celite. The crude product from evaporation of the filtrate was chromatographed on silica gel eluting with 80% ethyl acetate in hexane and the 5-bromo-2-(1-hydroxy-1-methylethyl)pyridine N-oxide compound was obtained as a white solid.

### Step 3: 1-(3-bromophenyl)-1,4-dihydro[1,8]naphthyridin-4-one-3-carboxamide

Following the procedure of Step 4 of Example 1C but substituting 28% aqueous ammonium hydroxide for isopropylamine the 1-(3-bromophenyl)-1,4-dihydro[1,8]naphthyridin-4-one-3-carboxamide compound was obtained as a solid.

### Step 4: 1-{3-[6-(1-hydroxy-1-methylethyl)-1-oxidopyridin-3-yl]phenyl-1,4-dihydro[1,8]naphthyridin-4-one-3-carboxamide

Following the procedure of Example 24C but substituting 5-bromo-2-(1-hydroxy-1-methylethyl)pyridine N-oxide from Step 2 above for ethyl 5-bromonicotinate and 1-(3-bromophenyl)-1,4-dihydro[1,8]naphthyridin-4-one-3-carboxamide for N-isopropyl-1-(3-bromophenyl)-1,4-dihydro[1,8]naphthyridin-4-one-3-carboxamide, the 1-{3-[6-(1-hydroxy-1-methylethyl)-1-oxidopyridin-3-yl]phenyl}-1,4-dihydro[1,8]naphthyridin-4-one-3-carboxamide compound was obtained as a solid.

¹H NMR (CDCl₃) δ 1.76 (s, 6H), 5.83 (br, 1H, NH). 7.50 (d, 1H), 7.55 (m, 1H), 7.57-7.62 (m, 2H), 7.65 (m, 2H), 7.72-7.78 (m, 2H), 8.55 (s, 1H, OH), 8.75 (m, 1H), 8.90 (dd, 1H), 9.08 (s, 1H), 9.52 (br, 1H, NH).

### Example 31C

### N-Isopropyl-1-{3-[4-(pyridin-3-yl)phenyl]phenyl}-1,4-dihydro[1,8]naphthyridin-4-one-3-carboxamide

### Step 1: N-Isopropyl-1-[3-(4,4,5,5,-tetramethyl-1,3,2-dioxaborolan-2-yl)phenyl]-1,4-dihydro[1,8]naphthyridin-4-one-3-carboxamide

A mixture of N-isopropyl-1-(3-bromophenyl)-1,4-dihydro[1,8]naphthyridin-4-one-3-carboxamide from Step 4 of Example 1C, diboron pinacol ester (1.1 eq), potassium acetate (3.5 eq) and [1,1′-bis (diphenylphosphino)ferrocene]dichloropalladium(II) (0.05 eq) in N,N-dimethylformamide (5 ml/mmol) was stirred at 85° C. for 18 hours. A further amount of diboron pinacol ester (0.4 eq) and palladium catalyst (0.05 eq) were added and heating and stirring were continued for a further 24 hours. After cooling, the mixture was partitioned between ethyl acetate and water, and the crude product from the organic phase was chromatographed on silica gel eluting with a 1:1 mixture of ethyl acetate and hexane. The product was then stirred in hexane at room temperature for several hours and filtered to afford the N-Isopropyl-1-[3-(4,4,5,5,-tetramethyl-1,3,2-dioxaborolan-2-yl)phenyl]-1,4-dihydro[1,8]naphthyridin-4-one-3-carboxamide compound as a white solid.

### Step 2: 3-(4-Bromophenyl)pyridine

A mixture of pyridine-3-boronic acid 1,3-propanediol cyclic ester, 4-bromoiodobenzene (1.1 eq), [1,11′-bis (diphenylphosphino)ferrocene]dichloropalladium(II) (0.05 eq) and 2M aqueous sodium carbonate (5 eq) in N,N-dimethylformamide (2 ml/mmol) was stirred at 85° C. for 4 hours. After quenching with saturated aqueous ammonium chloride solution, the mixture was partitioned between ethyl acetate and water, and the crude product from the organic phase was chromatographed on silica gel eluting with a 1:9 mixture of ethyl acetate and hexane to afford the 3-(4-Bromophenyl)pyridine compound as a solid.

### Step 3: N-Isopropyl-1-1, 3-[4-(pyridin-3-yl)phenyl]phenyl-1,4-dihydro[1,8]naphthyridin-4-one-3-carboxamide

A mixture of the boronate from Step 1,3-(4-bromophenyl)pyridine from Step 2 (1.5 eq), [1,1′-bis (diphenylphosphino)ferrocene]dichloropalladium(II) (0.05 eq) and 2M aqueous sodium carbonate (5 eq) in N,N-dimethylformamide (7 ml/mmol) was stirred at 85° C. for 1 hour. After cooling, the mixture was partitioned between ethyl acetate and water. The crude product from the organic phase was chromatographed on silica gel eluting with a 7:3 mixture of ethyl acetate and methylene chloride to afford the N-Isopropyl-1-{3-[4-(pyridin-3-yl)phenyl]phenyl}-1,4-dihydro[1,8]naphthyridin-4-one-3-carboxamide compound as a solid.

¹H NMR (CDCl₃) δ 1.30 (d, 6H), 4.25 (m, 1H), 7.35 (m, 1H), 7.39-7.48 (m, 2H), 7.60-7.75 (m, 6H), 7.80 (d, 1H), 7.90 (d, 1H), 8.58 (d, 1H), 8.70 (m, 1H), 8.82 (d, 1H), 8.88 (s, 1H), 9.08 (s, 1H), 9.68 (br, NH).

### EXAMPLE 32C

### N-Cyclopropyl-1-[3-(5-methylsulfonylperidin-3-yl)]phenyl]-1,4-dihydro[1,8]naphthyridin-4-one-3-carboxamide

### Step 1: N-Cyclopropyl-1-[3-(4,4,5,5,-tetramethyl-1,3,2-dioxaborolan-2-yl)phenyl]-1,4-dihydro[1,8]naphthyridin-4-one-3-carboxamide

Following the procedure of Step 1 of Example 31C but substituting N-cyclopropyl-1-(3-bromophenyl)-1,4-dihydro[1,8]naphthyridin-4-one-3-carboxamide from step 1 of example 14 for N-isopropyl-1-(3-bromophenyl)-1,4-dihydro[1,8]naphthyridin-4-one-3-carboxamide the N-Cyclopropyl-1-[3-(4,4,5,5,-tetramethyl-1,3,2-dioxaborolan-2-yl)phenyl]-1,4-dihydro[1,8]naphthyridin-4-one-3-carboxamide compound was obtained as a white solid.

### Step 2: 3-Bromo-5-methylsulfonylpyridine

To 3,5-dibromopyridine (2.96 g, 12.5 mmol) in diethyl ether (70 ml) at −78° C. was added n-butyllithium 1.6M in hexanes (8.6 ml, 13.7 mmol) and the resulting mixture was stirred in the cold for 3 hours. Dimethyl disulfide (1.12 ml, 12.5 mmol) was added and the mixture was warmed to room temperature, then partitioned between ether and water. To the crude product from evaporation of the organic phase was added tetrahydrofuran (80 ml), methanol (20 ml), oxone (17 g) and enough saturated aqueous sodium bicarbonate to afford a slightly basic medium. After stirring for 4 hours at room temperature, an excess of 1M aqueous sodium metabisulfite was added, the organic solvents were evaporated, and the residue was partitioned between ethyl acetate and water. The crude product from the organic phase was stirred in a small volume of ethyl acetate and filtered to afford the 3-Bromo-5-methylsulfonylpyridine compound as a solid.

### Step 3: N-Cyclopropyl-1-[3-(5-methylsulfonylpyridin-3-yl)]phenyl]-1,4-dihydro[1,8]naphthyridin-4-one-3-carboxamide

Following the procedure of Step 3 of Example 31C but substituting 3-bromo-5-methylsulfonylpyridine from Step 2 above for 3-(4-bromophenyl)pyridine, and N-cyclopropyl-1-[3-(4,4,5,5,-tetramethyl-1,3,2-dioxaborolan-2-yl)phenyl]-1,4-dihydro[1,8]naphthyridin-4-one-3-carboxamide from Step 1 for N-isopropyl-1-[3-(4,4,5,5,-tetramethyl-1,3,2-dioxaborolan-2-yl)phenyl]-1,4-dihydro[1,8]naphthyridin-4-one-3-carboxamide, the N-Cyclopropyl-1-[3-(5-methylsulfonylpyridin-3-yl)]phenyl]-1,4-dihydro[1,8]naphthyridin-4-one-3-carboxamide compound was obtained as a solid.

¹H NMR (CDCl₃) δ 0.71 (m, 2H), 0.90 (m, 2H), 3.03 (m, 1H), 3.21 (s, 3H), 7.53 (m, 1H), 7.60 (d, 1H), 7.74 (s, 1H), 7.80 (t, 1H), 7.86 (d, 1H), 8.45 (m, 1H), 8.74 (m, 1H), 8.86 (d, 1H), 9.09 (s, 1H), 9.20 (d, 2H), 9.78 (br, NH).

### EXAMPLE 33C

### N-Cyclopropyl-1-{3-[4-(1-hydroxy-1-methylethyl)-1-oxidopyridin-2-yl]phenyl}-1,4-dihydro[1,8]naphthyridin-4-one-3-carboxamide

### Step 1: Methyl 2-bromoisonicotinate

To a solution of 2-bromoisonicotinic acid (Chem. Pharm. Bull., 38:2446 (1990)) (2.0 g) in tetrahydrofuran (100 ml) was added excess ethereal diazomethane and the resulting mixture was stirred at room temperature for 1 hour. The mixture was evaporated and the product chromatographed on silica gel eluting with a 1:3 mixture of ethyl acetate and hexane to afford the Methyl 2-bromoisonicotinate ester as a colorless liquid.

### Step 2: 2-Bromo-4-(1-hydroxy-1-methylethyl)pyridine

Following the procedure of Step 1 of Example 25C, but substituting methyl 2-bromoisonicotinate from Step 1 for ethyl 5-bromonicotinate, the 2-Bromo-4-(1-hydroxy-1-methylethyl)pyridine compound was obtained as a white solid.

### Step 3: 2-Bromo-4-(1-hydroxy-1-methylethyl)pyridine-N-oxide

Following the procedure of Step 2 of Example 30C but substituting 2-bromo-4-(1-hydroxy-1-methylethyl)pyridine from Step 2 for 5-bromo-2-(1-hydroxy-1-methylethyl) pyridine the 2-Bromo-4-(1-hydroxy-1-methylethyl)pyridine-N-oxide compound was obtained as a white solid.

### Step 4: N-Cyclopropyl-1-{3-[4-(1-hydroxy-1-methylethyl)-1-oxidopyridin-2-yl]phenyl}-1,4-dihydro[1,8]naphthyridin-4-one-3-carboxamide

Following the procedure of Step 3 of Example 32C, but substituting 2-bromo-4-(1-hydroxy-1-methylethyl)pyridine-N-oxide from Step 3 for 3-bromo-5-methylsulfonylpyridine, the N-Cyclopropyl-1-{3-[4-(1-hydroxy-1-methylethyl)-1-oxidopyridin-2-yl]phenyl}-1,4-dihydro[1,8]naphthyridin-4-one-3-carboxamide compound was obtained as a beige solid.

¹H NMR (DMSO-d₆), 0.57 (m, 2H), 0.79 (m, 2H), 1.45 (s, 6H), 2.90 (m, 1H), 5.35 (s, 1H, OH), 7.48 (m, 1H), 7.64 (m, 1H), 7.72 (m, 3H), 8.11 (m, 2H), 8.30 (d, 1H), 8.72 (dd, 1H), 8.78 (m, 1H), 8.82 (s, 1H), 9.72 (br, NH).

### EXAMPLE 34C

### N-Cyclopropyl-1-{3-[5-(1-hydroxy-1-methylethyl)pyridin-2-yl]phenyl}1,4-dihydro[1,8]naphthyridin-4-one-3-carboxamide

### Step 1: 2-Bromo-5-(1-hydroxy-1-methylethyl)pyridine

A solution of 2,5-dibromopyridine in diethyl ether (5 ml/mmol) was cooled to −78° C., and n-butyllithium 2.5M in hexanes (1.05 eq) was added slowly. After 2 h in the cold, acetone (1.3 eq) was added and stirring was continued for 1 hour. The resulting mixture was quenched with saturated aqueous ammonium chloride solution, warmed to room temperature, and partitioned between ether and water. The crude product from the organic phase was triturated with 1:1 ether-hexane and filtered to afford the 2-Bromo-5-(1-hydroxy-1-methylethyl)pyridine compound as a solid.

### Step 2: N-Cyclopropyl-1-{3-[5-(1-hydroxy-1-methylethyl)pyridin-2-yl]phenyl}-1,4-dihydro[1,8]naphthyridin-4-one-3-carboxamide

Following the procedure of Step 3 of Example 32C, but substituting 2-bromo-5-(1-hydroxy-1-methylethyl)pyridine from Step 1 for 3-bromo-5-methylsulfonylpyridine, the N-Cyclopropyl-1-{3-[5-(1-hydroxy-1-methylethyl)pyridin-2-yl]phenyl}-1,4-dihydro[1,8]naphthyridin-4-one-3-carboxamide compound was obtained as a solid.

¹H NMR (CDCl₃) δ 0.71 (m, 2H), 0.90 (m, 2H), 1.68 (s, 6H), 1.85 (s, 1H, OH), 3.04 (m, 1H), 7.45-7.52 (m, 2H), 7.71 (t, 1H), 7.79 (d, 1H), 7.95 (dd, 1H), 8.16 (s, 1H), 8.20 (d, 1H), 8.72 (m, 1H), 8.80-8.87 (m, 2H), 9.12 (s, 1H), 9.82 (br, NH).

### EXAMPLE 35C

### N-Cyclopropyl 1-{3-[3-(1-hydroxy-1-methylethyl)pyridin-4-yl]phenyl}-1,4-dihydro[1,8]naphthyidin-4-one-3-carboxamide

### Step 1: 4-Bromo-2-(1-hydroxy-1-methylethyl)pyridine

Following the sequence described in Steps 1-2 of Example 33C, but substituting 4-bromopicolinic acid (Aust. J. Chem. 24:390 (1971)) for 2-bromoisonicotinic acid in Step 1, the 4-Bromo-2-(1-hydroxy-1-methylethyl)pyridine compound was obtained as a white solid.

### Step 2: N-Cyclopropyl-1-{3-[3-(1-hydroxy-1-methylethyl)pyridin-4-yl]phenyl}-1,4-dihydro[1,8]naphthyridin-4-one-3-carboxamide

Following the procedure of Step 3 of Example 32C, but substituting 4-bromo-2-(1-hydroxy-1-methylethyl)pyridine from Step 1 for 3-bromo-5-methylsulfonylpyridine, the N-Cyclopropyl-1-{3-[3-(1-hydroxy-1-methylethyl)pyridin-4-yl]phenyl}-1,4-dihydro[1,8]naphthyridin-4-one-3-carboxamide compound was obtained as a beige solid.

¹H NMR (DMSO-d₆) δ 0.57 (m, 2H), 0.78 (m, 2H), 1.48 (s, 6H), 2.91 (m, 1H), 5.27 (s, 1H, OH), 7.62-7.66 (m, 2H), 7.72 7.79 (m, 2H), 8.01 (m, 1H), 8.10 (s, 1H), 8.58 (d, 1H), 8.73-8.79 (m, 2H), 8.84 (s, 1H), 9.73 (br, NH).

### EXAMPLE 36C

### Synthesis of N-Cyclopropyl-1-{3-[3-(1-hydroxy-1-methylethyl)-1-oxidopyridin-4-yl]phenyl}-1,4-dihydro[1,8]naphthyridin-4-one-3-carboxamide

### Step 1: 4-Bromo-2-(1-hydroxy-1-methylethyl)pyridine N-oxide

Following the procedure of Step 2 of Example 30C, but substituting 4-bromo-2-(1-hydroxy-1-methylethyl)pyridine from Step 1 of Example 35C for 5-bromo-2-(1-hydroxy-1-methylethyl)pyridine, the 4-Bromo-2-(1-hydroxy-1-methylethyl)pyridine N-oxide compound was obtained as a white solid.

### Step 2: N-Cyclopropyl-1-{3-[3-(1-hydroxy-1-methylethyl)-1-oxidopyridin-4-yl]phenyl}-1,4-dihydro[1,8]naphthyridin-4-one-3-carboxamide

Following the procedure of Step 3 of Example 32C, but substituting 4-bromo-2-(1-hydroxy-1-methylethyl)pyridine-N-oxide from Step 1 for 3-bromo-5-methylsulfonylpyridine, the N-Cyclopropyl-1-{3-[3-(1-hydroxy-1-methylethyl)-1-oxidopyridin-4-yl]phenyl}-1,4-dihydro[1,8]naphthyridin-4-one-3-carboxamide compound was obtained as a beige solid.

¹H NMR (DMSO-d₆) δ 0.57 (m, 2H), 0.78 (m, 2H), 1.62 (s, 6H), 2.90 (m, 1H), 6.99 (s, 1H, OH), 7.65-7.84 (m, 4H), 7.94 (s, 1H), 8.03 (dd, 1H), 8.15 (s, 1H), 8.38 (d, 1H), 8.73-8.78 (m, 2H), 8.83 (s, 1H), 9.73 (br, NH).

### EXAMPLE 37C

### N-Cyclopropyl-1-[3-(6-isopropylsulfonylpyridin-3-yl)]phenyl]-1,4-dihydro[1,8]naphthyridin-4-one-3-carboxamide

### Step 1: 5-Bromo-2-isopropylthiopyridine

To a mixture of 2,5-dibromopyridine (2.07 g, 8.73 mmol) and 2-propanethiol (0.97 ml, 10.4 mmol) in N,N-dimethylformamide (20 ml) at 0° C. was added portionwise sodium hydride 60% dispersed in oil (450 mg, 11.3 mmol). The resulting mixture was stirred at room temperature for 1 hour, then partitioned between ether and water. The crude product from the organic phase was chromatographed on silica gel eluting with 10% ethyl acetate in hexane to afford the 5-Bromo-2-isopropylthiopyridine compound as a solid.

### Step 2: 5-Bromo-2-isopropylsulfonylpyridine

To a solution of 5-bromo-2-isopropylthiopyridine from Step 1 (2.03 g, 8.75 mmol) in tetrahydrofuran (50 ml) and methanol (25 ml) at 0° C. was added oxone (15.8 g, 25.8 mmol) and then saturated aqueous sodium bicarbonate (25 ml). The resulting mixture was stirred at room temperature for 6 hours. The mixture was quenched with aqueous sodium bicarbonate and partitioned between ethyl acetate and water. The crude product from the organic phase was chromatographed on silica gel eluting with 20% ethyl acetate in hexane to afford the 5-Bromo-2-isopropylsulfonylpyridine compound as a white solid.

### Step 3: N-Cyclopropyl-1-[3-(6-isopropylsulfonylpyridin-3-yl)]phenyl]-1,4-dihydro[1,8]naphthyridin-4-one-3-carboxamide

Following the procedure of Step 3 of Example 32C, but substituting 5-bromo-2-isopropylsulfonylpyridine from Step 2 for 3-bromo-5-methylsulfonylpyridine, the N-Cyclopropyl-1-[3-(6-isopropylsulfonylpyridin-3-yl)]phenyl]-1,4-dihydro[1,8]naphthyridin-4-one-3-carboxamide compound was obtained as a solid.

¹H NMR (CDCl₃) δ 0.70 (m, 2H), 0.89 (m, 2H), 1.39 (d, 6H), 3.00 (m, 1H), 3.82 (m, 1H), 7.51 (m, 1H), 7.60 (d, 1H), 7.72 (s, 1H), 7.80 (t, 1H), 7.83 (d, 1H), 8.15-8.24 (m, 2H), 8.72 (m, 1H), 8.86 (dd, 1H), 9.03 (s, 1H), 9.10 (s, 1H), 9.77 (br, NH).

### EXAMPLE 38C

### N-Cyclopropyl-1-[3-(6-methoxypyridin-3-yl)phenyl]-1,4-dihydro[1,8]naphthyridin-4-one-3-carboxamide

### Step 1: 5-Bromo-2-methoxypyridine

To a solution of 2,5-dibromopyridine (6.95 g, 29 mmol) in N,N-dimethylformamide (5 ml) was added methanol (3.56 ml) and 1M potassium tert-butoxide (32.3 ml) and the resulting mixture was stirred at room temperature for 18 hours. The resulting slurry was quenched with saturated aqueous ammonium chloride solution and partitioned between ethyl acetate and water. The crude product from the organic phase was chromatographed on silica gel eluting with a 1:9 mixture of ether and hexane to afford the 5-Bromo-2-methoxypyridine compound as an oil.

### Step 2: N-Cyclopropyl-1-[3-(6-methoxypyridin-3-yl)phenyl]-1,4-dihydro[1,8]naphthyridin-4-one-3-carboxamide

Following the procedure of Step 3 of Example 32C, but substituting 5-bromo-2-methoxypyridine from Step 1 for 3-bromo-5-methylsulfonylpyridine, the N-cyclopropyl-1-[3-(6-methoxypyridin-3-yl)phenyl]-1,4-dihydro[1,8]naphthyridin-4-one-3-carboxamide compound was obtained as a solid.

¹H NMR (CDCl₃) δ 0.71 (m, 2H), 0.89 (m, 2H), 3.00 (m, 1H), 4.00 (s, 3H), 6.85 (d, 1H), 7.44 (d, 1H), 7.50 (m, 1H), 7.62 (s, 1H), 7.68 (t, 1H), 7.73 (d, 1H), 7.83 (dd, 1H), 8.44 (s, 1H), 8.73 (m, 1H), 8.85 (dd, 1H), 9.10 (s, 1H), 9.82 (br, NH).

### EXAMPLE 39C

### N-Cyclopropyl-1-[3-(6-methylpyridin-3-yl)phenyl]-1,4-dihydro[1,8]naphthyridin-4-one-3-carboxamide

Following the procedure of Step 3 of Example 32C, but substituting 5-bromo-2-methylpyridine from Step 1 of Example 28C for 3-bromo-5-methylsulfonylpyridine, the title compound was obtained as a solid.

¹H NMR (CDCl₃) δ 0.72 (m, 2H), 0.90 (m, 2H), 2.65 (s, 3H), 3.03 (m, 1H), 7.28 (d, 1H), 7.45-7.53 (m, 2H), 7.66 (s, 1H), 7.72 (t, 1H), 7.80 (d, 1H), 7.84 (dd, 1H), 8.73 (m, 1H), 8.80 (s, 1H), 8.86 (dd, 1H), 9.11 (s, 1H), 9.82 (br, NH).

### EXAMPLE 40C

### N-Cyclopropyl-1-{3-[6-(2,2,2-trifluoroethoxy)pyridin-3-yl]phenyl}-1,4-dihydro [1,8]naphthyridin-4-one-3-carboxamide

### Step 1: 5-Bromo-2-(2,2,2-trifluoroethoxy)pyridine

Following the procedure of Step 1 of Example 38C, but substituting 2,2,2-trifluoroethanol for methanol, with heating at 70° C. for 18 hours, the 5-Bromo-2-(2,2,2-trifluoroethoxy)pyridine compound was obtained as an oil.

### Step 2: N-Cyclopropyl-1-{3-[6-(2,2,2-trifluoroethoxy)pyridin-3-yl]phenyl}-1,4-dihydro[1,8]naphthyridin-4-one-3-carboxamide

Following the procedure of Step 3 of Example 32C, but substituting 5-bromo-2-(2,2,2-trifluoroethoxy)pyridine from Step 1 for 3-bromo-5-methylsulfonylpyridine, the N-Cyclopropyl-1-{3-[6-(2,2,2-trifluoroethoxy)pyridin-3-yl]phenyl}-1,4-dihydro[1,8]naphthyridin-4-one-3-carboxamide compound was obtained as a solid.

¹H NMR (CDCl₃) δ 0.72 (m, 2H), 0.90 (m, 2H), 3.03 (m, 1H), 4.85 (q, 2H), 7.00 (d, 1H), 7.43-7.53 (m, 2H), 7.62 (s, 1H), 7.69-7.78 (m, 2H), 7.92 (dd, 1H), 8.42 (s, 1H), 8.73 (m, 1H), 8.85 (dd, 1H), 9.10 (s, 1H), 9.80 (br, NH).

### EXAMPLE 41C

### N-Cyclopropyl-1-[3-(5-bromopyridin-3-yl)phenyl]-1,4-dihydro[1,8]naphthyridin-4-one-3-carboxamide

Following the procedure of Step 3 of Example 32C, but substituting 3,5-dibromopyridine for 3-bromo-5-methylsulfonylpyridine, the title compound was obtained as a white solid.

¹H NMR (DMSO-d₆) δ 0.58 (m, 2H), 0.79 (m, 2H), 2.90 (m, 1H), 7.65 (m, 1H), 7.71-7.77 (m, 2H), 8.03 (d, 1H), 8.14 (s, 1H), 8.49 (s, 1H), 8.74 (brs, 1H), 8.79 (brs, 1H), 8.86 (s, 1H), 9.01 (s, 1H), 9.73 (br, NH).

### EXAMPLE 42C

### N-Cyclopropyl-1-[3-(6-benzyloxypyridin-3-yl)phenyl]-1,4-dihydro[1,8]naphthyridin-4-one-3-carboxamide

### Step 1: 2-Benzyloxy-5-bromopyridine

A mixture of 2,5-dibromopyridine, benzyl alcohol (1.3 eq), potassium hydroxide pellets (2.4 eq) and dibenzo-18-crown-6 (0.05 eq) in toluene (4 ml/mmol) was refluxed with azeotropic removal of water for 3 hours. After evaporation of the toluene, the resulting mixture was partitioned between chloroform and water. The crude product from the organic phase was recrystallized from ether-hexane to afford the 2-Benzyloxy-5-bromopyridine compound as a solid.

### Step 2: N-Cyclopropyl-1-[3-(6-benzyloxypyridin-3-yl)phenyl]-1,4-dihydro[1,8]naphthyridin-4-one-3-carboxamide

Following the procedure of Step 3 of Example 32C, but substituting 2-benzyloxy-5-bromopyridine from Step 1 for 3-bromo-5-methylsulfonylpyridine, the N-Cyclopropyl-1-[3-(6-benzyloxypyridin-3-yl)phenyl]-1,4-dihydro[1,8]naphthyridin-4-one-3-carboxamide compound was obtained as a white solid.

¹H NMR (DMSO-d₆) δ 0.57 (m, 2H), 0.79 (m, 2H), 2.91 (m, 1H), 5.42 (s, 2H) 7.00 (d, 1H), 7.32-7.48 (m, 5H), 7.61-7.72 (m, 3H), 7.90 (d, 1H), 7.99 (s, 1H), 8.14 (d, 1H), 8.59 (s, 1H), 8.73-8.84 (m, 3H), 9.73 (br, NH).

### EXAMPLE 43C

### N-Cyclopropyl-1-{3-[6-dicyclopropyl(hydroxy)methyl-1-oxidopyridin-3-yl]phenyl}-1,4-dihydro[1,8]naphthyridin-4-one-3-carboxamide

### Step 1: 5-Bromo-2-dicyclopropyl(hydroxy)methylpyridine N-oxide

Following the procedure of Steps 1 and 2 of Example 30C, but substituting dicyclopropyl ketone for acetone in Step 1, the 5-Bromo-2-dicyclopropyl(hydroxy)methylpyridine N-oxide compound was obtained as a solid.

### Step 2: N-Cyclopropyl-1-{3-[6-dicyclopropyl(hydroxy)methyl-1-oxidopyridin-3-yl]phenyl}-1,4-dihydro[1,8]naphthyridin-4-one-3-carboxamide

Following the procedure of Step 3 of Example 32C, but substituting 5-bromo-2-dicyclopropyl(hydroxy)methylpyridine N-oxide from Step 1 for 3-bromo-5-methylsulfonylpyridine, the N-Cyclopropyl-1-{3-[6-dicyclopropyl(hydroxy)methyl-1-oxidopyridin-3-yl]phenyl}-1,4-dihydro[1,8]naphthyridin-4-one-3-carboxamide compound was obtained as a white solid.

¹H NMR (CDCl₃) δ 0.52 (m, 4H), 0.70 (m, 4H), 0.76 (m, 2H), 0.89 (m, 2H), 1.35 (m, 2H), 3.02 (m, 1H), 7.52 (m, 1H), 7.58 (m, 1H), 7.62 (dd, 1H), 7.68 (s, 1H), 7.73-7.80 (m, 3H), 8.15 (br, 1H, OH), 8.49 (s, 1H), 8.72 (m, 1H), 8.85 (dd, 1H), 9.09 (s, 1H), 9.78 (br, NH).

### EXAMPLE 44C

### N-Cyclopropyl-1-{3-[5-(1-hydroxy-1-methylethyl)-1-oxidopyridin-2-yl]phenyl}-1,4-dihydro[1,8]naphthyridin-4-one-3-carboxamide

### Step 1: 2-Bromo-5-(1-hydroxy-1-methylethyl)pyridine N-oxide

Following the procedure of Step 2 of Example 30, but substituting 2-bromo-5-(1-hydroxy-1-methylethyl)pyridine from Step 1 of Example 34C for 5-bromo-2-(1-hydroxy-1-methylethyl)pyridine, the 2-Bromo-5-(1-hydroxy-1-methylethyl)pyridine N-oxide compound was obtained as a white solid.

### Step 2: N-Cyclopropyl-1-{3-[5-(1-hydroxy-1-methylethyl)-1-oxidopyridin-2-yl]phenyl}-1,4-dihydro[1,8]naphthyridin-4-one-3-carboxamide

Following the procedure of Step 3 of Example 32C, but substituting 2-bromo-5-(1-hydroxy-1-methylethyl)pyridine N-oxide from Step 1 for 3-bromo-5-methylsulfonylpyridine, the N-Cyclopropyl-1-{3-[5-(1-hydroxy-1-methylethyl)-1-oxidopyridin-2-yl]phenyl}-1,4-dihydro[1,8]naphthyridin-4-one-3-carboxamide compound was obtained as a solid.

¹H NMR (CDCl₃) δ 0.69 (m, 2H), 0.88 (m, 2H), 1.63 (s, 6H), 2.20 (s, 1H, OH), 2.98 (m, 1H), 7.38-7.49 (m, 3H), 7.52 (d, 1H), 7.70 (t, 1H), 7.98-8.04 (m, 2H), 8.50 (s, 1H), 8.69 (m, 1H), 8.80 (dd, 1H), 9.08 (s, 1H), 9.75 (br, NH).

### EXAMPLE 45C

### N-Cyclopropyl-1-{3-[6-(1-hydroxy-1-methylethyl)pyridin-3-yl]phenyl}-1,4-dihydro[1,8]naphthyridin-4-one-3-carboxamide

Following the procedure of Step 3 of Example 32C, but substituting 5-bromo-2-(1-hydroxy-1-methylethyl)pyridine from Step 1 of Example 30C for 3-bromo-5-methylsulfonylpyridine, the title compound was obtained as a solid.

¹H NMR (CDCl₃) δ 0.72 (m, 2H), 090 (m, 2H), 1.62 (s, 6H), 3.02 (m, 1H), 4.85 (s, 1H, OH), 7.48-7.53 (m, 3H), 7.68 (s, 1H), 7.73 (t, 1H), 7.80 (d, 1H), 7.95 (dd, 1H), 8.72 (m, 1H), 8.81 (s, 1H), 8.86 (dd, 1H), 9.10 (s, 1H), 9.78 (br, NH).

### EXAMPLE 46C

### N-Isobutyl-1-{3-[6-(1-hydroxy-1-methylethyl)pyridin-3-yl]phenyl}-1,4-dihydro [1,8]naphthyridin-4-one-3-carboxamide

### Step 1: 5-(3-Aminophenyl)-2-(1-hydroxy-1-methylethyl)pyridine

Following the procedure of Step 5 of Example 1C, but substituting 3-aminophenylboronic acid for 3-acetyl phenylboronic acid and 5-bromo-2-(1-hydroxy-1-methylethyl)pyridine from Step 1 of Example 30C for N-isopropyl-1-(3-bromophenyl)-1,4-dihydro[1,8]naphthyridin-4-one-3-carboxamide, the 5-(3-Aminophenyl)-2-(1-hydroxy-1-methylethyl)pyridine compound was obtained as a solid.

### Step 2: 1-{3-[6-(1-Hydroxy-1-methylethyl)pyridin-3-yl]phenyl}-1,4-dihydro[1,8]naphthyridin-4-one-3-carboxylic acid

Following the procedures of Steps 1-3 of Example 1C, but substituting 5-(3-aminophenyl)-2-(1-hydroxy-1-methylethyl)pyridine for 3-bromoaniline from Step 1 in the First Step, the 1-{3-[6-(1-hydroxy-1-methylethyl)pyridin-3-yl]phenyl}-1,4-dihydro[1,8]naphthyridin-4-one-3-carboxylic acid compound was obtained as a solid.

### Step 3: N-Isobutyl-1-{3-[6-(1-hydroxy-1-methylethylpyridin-3-yl]phenyl}-1,4-dihydro[1,8]naphthyridin-4-one-3-carboxamide

Following the procedure of Step 4 of Example 1C, but substituting the acid from Step 2 for 1-(3-bromophenyl)-1,4-dihydro[1,8]naphthyridin-4-one-3-carboxylic acid, and isobutylamine for isopropylamine, the N-Isobutyl-1-{3-[6-(1-hydroxy-1-methylethyl)pyridin-3-yl]phenyl}-1,4-dihydro[1,8]naphthyridin-4-one-3-carboxamide compound was obtained as a cream-colored solid.

¹H NMR (Acetone-d₆) δ 0.98 (d, 6H), 1.53 (s, 6H), 1.88 (m, 1H), 3.26 (t, 2H), 4.66 (s, 1H, OH), 7.60 (m, 1H), 7.69 (d, 1H), 7.76-7.79 (m, 2H), 7.95 (d, 1H), 8.05 (s, 1H), 8.16 (dd, 1H), 8.73 (m, 1H), 8.79 (dd, 1H), 8.90 (s, 1H), 8.94 (s, 1H), 9.83 (br, NH).

### EXAMPLE 47C

### N-Cyclopropyl-1-{5-bromo-3-[6-(1-hydroxy-1-methylethyl)pyridin-3-yl]phenyl}-1,4-dihydro[1,8]naphthyridin-4-one-3-carboxamide

### Step 1: 1-(3,5-Dibromophenyl)-1,4-dihydro[1,8]naphthyridin-4-one-3-carboxylic acid

Following the procedures of Steps 1-3 of Example 1C, but substituting 3,5-dibromoaniline for 3-bromoaniline in Step 1, the 1-(3,5-Dibromophenyl)-1,4-dihydro[1,8]naphthyridin-4-one-3-carboxylic acid compound was obtained as a beige solid.

### Step 2: N-Cyclopropyl-1-(3,5-dibromophenyl)-1,4-dihydro[1,8]naphthyridin-4-one-3-carboxamide

Following the procedure of Step 4 of Example 1C, but substituting the 1-(3,5-Dibromophenyl)-1,4-dihydro[1,8]naphthyridin-4-one-3-carboxylic acid from Step 1 for 1-(3-bromophenyl)-1,4-dihydro[1,8]naphthyridin-4-one-3-carboxylic acid, and cyclopropylamine for isopropylamine, the N-Cyclopropyl-1-(3,5-dibromophenyl)-1,4-dihydro[1,8]naphthyridin-4-one-3-carboxamide compound was obtained as a solid.

### Step 3: 2-(1-Hydroxy-1-methylethyl)-5-tributylstannylpyridine

To a suspension of 2,5-dibromopyridine in toluene (5 ml/mmol) at −78° C., was added n-butyllithium 2.5M in hexanes (1 eq) and the resulting mixture was stirred in the cold for 2.5 hours. Acetone (1 eq) was added, and the mixture was warmed to −50° C. and became a brown solution. After cooling down to −78° C., more n-butyllithium (1 eq) was added along with ether (2 ml/mmol). After stirring in the cold for a further hour, tributyltin chloride (1.1 eq) was added and the mixture was warmed to room temperature and stirred for 2 hours. The mixture was quenched with saturated aqueous ammonium chloride solution and partitioned between ethyl acetate and water. The crude product from the organic phase was chromatographed on silica gel eluting with a 1:9 mixture of ethyl acetate and hexane to afford the 2-(1-Hydroxy-1-methylethyl)-5-tributylstannylpyridine compound as a colorless liquid.

### Step 4: N-Cyclopropyl-1-{5-bromo-3-[6-(1-hydroxy-1-methylethyl)pyridin-3-yl]phenyl}-1,4-dihydro[1,8]naphthyridin-4-one-3-carboxamide

A mixture of N-cyclopropyl-1-(3,5-dibromophenyl)-1,4-dihydro[1,8]naphthyridin-4-one-3-carboxamide from Step 2,2-(1-hydroxy-1-methylethyl)-5-tributylstannylpyridine from Step 3 (1.4 eq), 1,1′-bis (diphenylphosphino)ferrocene]dichloro-palladium(II) (0.05 eq), and cuprous iodide (0.05 eq) in N,N-dimethylformamide (15 ml/mmol) was stirred at 85° C. for 5 hours. After cooling the resulting mixture was partitioned between ethyl acetate and water. The crude product from the organic phase was chromatographed on silica gel eluting with a 1:6:3 mixture of ethanol, ethyl acetate and methylene chloride to afford the N-Cyclopropyl-1-{5-bromo-3-[6-(1-hydroxy-1-methylethyl)pyridin-3-yl]phenyl}-1,4-dihydro[1,8]naphthyridin-4-one-3-carboxamide compound as a solid.

¹H NMR (CDCl₃) δ 0.72 (m, 2H), 0.90 (m, 2H), 1.62 (s, 6H), 3.02 (m, 1H), 4.76 (s, 1H, OH), 7.50-7.56 (m, 2H), 7.62 (s, 1H), 7.69 (s, 1H), 7.90-7.96 (m, 2H), 8.74 (m, 1H), 8.79 (s, 1H), 8.86 (dd, 1H), 9.07 (s, 1H), 9.74 (br, NH).

### EXAMPLE 48C

### N-Cyclopropyl-1-{3-[6-(1-hydroxy-1-methylethyl)pyridin-2-yl]phenyl}-1,4-dihydro [1,8]naphthyridin-4-one-3-carboxamide

### Step 1: 2-(1-Hydroxy-1-methylethyl)-6-tributylstannylpyridine

Following the procedure of Step 3 of Example 47C, but substituting 2,6-dibromopyridine for 2,5-dibromopyridine, the 2-(1-Hydroxy-1-methylethyl)-6-tributylstannylpyridine compound was obtained.

### Step 2: N-Cyclopropyl-1-{3-[6-(1-hydroxy-1-methylethyl)pyridin-2-yl]phenyl}-1,4-dihydro[1,8]naphthyridin-4-one-3-carboxamide

Following the procedure of Step 4 of Example 47C, but substituting 2-(1-hydroxy-1-methylethyl)-6 tributylstannylpyridine from Step 1 for 2-(1-hydroxy-1-methylethyl)-5-tributylstannylpyridine, the N-Cyclopropyl-1-{3-[6-(1-hydroxy-1-methylethyl)pyridin-2-yl]phenyl}-1,4-dihydro[1,8]naphthyridin-4-one-3-carboxamide compound was obtained as a solid.

¹H NMR (CDCl₃) δ 0.72 (m, 2H), 0.90 (m, 2H), 1.61 (s, 6H), 3.04 (m, 1H), 5.13 (s, 1H, OH), 7.40 (d, 1H), 7.46-7.53 (m, 2H), 7.70-7.76 (m, 2H), 7.85 (t, 1H), 8.13 (s, 1H), 8.22 (d, 1H), 8.73 (m, 1H), 8.87 (d, 1H), 9,12 (s, 1H), 9.83 (br, NH).

### EXAMPLE 49C

### N-Isopropyl-1-[3-(4-methylsulfonylphenyl)phenyl]-1,4-dihydro[1,8]naphthyridin-4-one-3-carboxamide

To a mixture of N-isopropyl-1-[3-(4-methylthiophenyl)phenyl]-1,4-dihydro[1,8]naphthyridin-4-one-3-carboxamide from Example 22C in tetrahydrofuran (24 ml/mmol), methanol (12 ml/mmol), and water (12 ml/mmol), was added oxone (2.24 eq) and the resulting mixture was stirred at room temperature for 2 hours. The mixture was quenched with saturated aqueous sodium bicarbonate and partitioned between ethyl acetate and water. The crude product from the organic phase was chromatographed on silica gel eluting with 30% ether in methylene chloride to afford the title compound as a white solid.

¹H NMR (Acetone-d₆) δ 1.25 (d, 6H), 3.16 (s, 3H), 4.18 (m, 1H), 7.60 (m, 1H), 7.74 (d, 1H), 7.79 (t, 1H), 7.99 (d, 1H), 8.05 (s, 4H), 8.09 (s, 1H), 8.72 (m, 1H), 8.78 (dd, 1H), 8.93 (s, 1H), 9.64 (br, NH).

### EXAMPLE 50C

### N-Cyclopropyl-1-[3-(6-methylsulfonylpyridin-3-yl)phenyl]-1,4-dihydro[1,8]naphthyridin-4-one-3-carboxamide

### Step 1: 5-Bromo-2-methylthiopyridine

A mixture of 2,5-dibromopyridine and sodium thiomethoxide (1.3 eq) in N,N-dimethylformamide (2 ml/mmol) was stirred at room temperature for 20 minutes then cooled to 0° C. After diluting with cold water the precipitate was filtered to afford the 5-Bromo-2-methylthiopyridine compound as a solid.

### Step 2: N-Cyclopropyl-1-[3-(6-methylthiopyridin-3-yl)phenyl]-1,4-dihydro[1,8]naphthyridin-4-one-3-carboxamide

Following the procedure of Step 3 of Example 32C, but substituting 5-bromo-2-methylthiopyridine from Step 1 for 3-bromo-5-methylsulfonylpyridine, the N-Cyclopropyl-1-[3-(6-methylthiopyridin-3-yl)phenyl]-1,4-dihydro[1,8]naphthyridin-4-one-3-carboxamide compound was obtained as a solid.

### Step 3: N-Cyclopropyl-1-[3-(6-methylsulfonylpyridin-3-yl)phenyl]-1,4-dihydro[1,8]naphthyridin-4-one-3-carboxamide

Following the procedure of Example 49C, but substituting N-cyclopropyl-1-[3-(6-methylthiopyridin-3-yl)phenyl]-1,4-dihydro[1,8]naphthyridin-4-one-3-carboxamide from Step 2 for N-isopropyl-1-[3-(4-methylthiophenyl)phenyl]-1,4-dihydro[1,8]naphthyridin-4-one-3-carboxamide, the N-Cyclopropyl-1-[3-(6-methylsulfonylpyridin-3-yl)phenyl]-1,4-dihydro[1,8]naphthyridin-4-one-3-carboxamide compound was obtained as a solid.

¹H NMR (CDCl₃) δ 0.66 (m, 2H), 0.84 (m, 2H), 2.97 (m, 1H), 3.26 (s, 3H), 7.48 (m, 1H), 7.55 (d, 1H), 7.67 (s, 1H), 7.74-7.80 (m, 2H), 8.14-8.19 (m, 2H), 8.68 (m, 1H), 8.81 (dd, 1H), 8.96 (s, 1H), 9.05 (s, 1H), 9.73 (br, NH).

### EXAMPLE 51C

### N-Isopropyl-1-[3-(5-methylsulfonylpyridin-3-yl)phenyl]-1,4-dihydro[1,8]naphthyridin-4-one-3-carboxamide

Following the procedure of Example 49C, but substituting N-isopropyl-1-[3-(5-methylthiopyridin-3-yl)phenyl]-1,4-dihydro[1,8]naphthyridin-4-one-3-carboxamide from Example 15C for N-isopropyl-1-[3-(4-methylthiophenyl)phenyl]-1,4-dihydro[1,8]naphthyridin-4-one-3-carboxamide, the title compound was obtained as a solid.

¹H NMR (CDCl₃) δ 1.33 (d, 6H), 3.20 (s, 3H), 4.31 (m, 1H), 7.52 (m, 1H), 7.60 (d, 1H), 7.73 (s, 1H), 7.79 (t, 1H), 7.86 (d, 1H), 8.48 (m, 1H), 8.73 (m, 1H), 8.88 (d, 1H), 9.08 (s, 1H), 9.19 (d, 2H), 9.68 (br, NH).

### EXAMPLE 52C

### N-Cyclopropyl-1-[3-(4-ethylsulfonylphenyl)phenyl]-1,4-dihydro[1,8]naphthyridin-4-one-3-carboxamide

Following the procedure of Example 49C, but substituting N-cyclopropyl-1-[3-(4-ethylthiophenyl)phenyl]-1,4-dihydro[1,8]naphthyridin-4-one-3-carboxamide from Example 18C for N-isopropyl-1-[3-(4-methylthiophenyl)phenyl]-1,4-dihydro[1,8]naphthyridin-4-one-3-carboxamide, the title compound was obtained as a solid.

¹H NMR (CDCl₃) δ 0.72 (m, 2H), 0.90 (m, 2H), 1.35 (t, 3H), 3.02 (m, 1H), 3.18 (q, 2H), 7.48-7.56 (m, 2H), 7.70 (s, 1H), 7.75 (t, 1H), 7.84 (m, 3H), 8.03 (d, 2H), 8.73 (m, 1H), 8.85 (dd, 1H), 9.10 (s, 1H), 9.80 (br, NH).

### EXAMPLE 53C

### N-Cyclopropyl-1-[3-(4-ethylsulfinylphenyl)phenyl]-1,4-dihydro[1,8]naphthyridin-4-one-3-carboxamide

To a solution of N-cyclopropyl-1-[3-(4-ethylthiophenyl)phenyl]-1,4-dihydro[1,8]naphthyridin-4-one-3-carboxamide from Example 18C, in a 1:1 mixture of methylene chloride and methanol (9 ml/mmol), was added at 0° C. magnesium monoperoxyphthalate hexahydrate (MMPP, 0.5 molareq) and the resulting mixture was stirred in the cold for 2 hours. The mixture was quenched with saturated aqueous sodium bicarbonate and partitioned between methylene chloride and water. The crude product from the organic phase was chromatographed on silica gel eluting with a 90:9:1 mixture of methylene chloride, ethanol and 28% aqueous ammonium hydroxide to afford the title compound as a solid.

¹H NMR (CDCl₃) δ 0.68 (m, 2H), 0.85 (m, 2H), 1.15 (m, 3H), 2.80 (m, 1H), 2.94 (m, 1H), 2.98 (m, 1H), 7.45-7.50 (m, 2H), 7.65-7.73 (m, 4H), 7.76-7.82 (m, 3H), 8.71 (m, 1H), 8.83 (dd, 1H), 9.06 (s, 1H), 9.78 (br, NH).

### EXAMPLE 54C

### N-Isopropyl-1-{3-[4-(1-oximidoethyl)phenyl]phenyl}-1,4-dihydro[1,8]naphthyridin-4-one-3-carboxamide

To a solution of N-isopropyl-1-[3-(4-acetylphenyl)phenyl]-1,4-dihydro[1,8]naphthyridin-4-one-3-carboxamide from Example 4C in pyridine (11 ml/mmol) at room temperature was added hydroxylamine hydrochloride (2.1 eq) and the resulting mixture was stirred for 16 hours. The mixture was filtered through celite and the filtrate evaporated. The residue was dissolved in ethyl acetate, washed with saturated aqueous sodium carbonate and then water, dried and evaporated. The residue was stirred in a small volume of acetone and filtered to afford the title compound as a solid.

¹H NMR (CDCl₃) δ 1.29 (d, 6H), 2.27 (s, 3H), 4.30 (m, 1H), 7.39 (d, 1H), 7.46 (m, 1H), 7.56 (d, 2H), 7.59-7.63 (m, 2H), 7.66 (d, 2H), 7.72 (d, 1H), 8.17 (s, 1H, OH), 8.69 (brs, 1H), 8.82 (d, 1H), 9.10 (s, 1H), 9.71 (br, NH).

### EXAMPLE 55C

### N-Isopropyl-1-{3-[4-(4-piperazin-1-yl)phenyl]-phenyl}-1,4-dihydro[1,8]naphthyridin-4-one-3-carboxamide

To a solution of N-isopropyl-1-{3-[4-(4-tertbutyloxycarbonylpiperazin-1-yl)phenyl)-phenyl}-1,4-dihydro[1,8]naphthyridin-4-one-3-carboxamide from Example 11C in methylene chloride (10 ml/mmol) was added trifluororacetic acid (6 ml/mmol) and the resulting mixture was stirred at room temperature for 2 hours, then warmed gently for 15 minutes. The mixture was evaporated and the crude product was chromatographed on silica gel eluting with a 9:0.9:0.1 mixture of methylene chloride, methanol and 28% aqueous ammonium hydroxide to afford the title compound as a solid.

¹H NMR (CDCl₃) δ 1.29 (d, 6H), 2.99 (m, 4H), 3.16 (m, 4H), 4.25 (m, 1H), 6.94 (d, 2H), 7.29 (d, 1H), 7.42 (m, 1H), 7.50 (d, 2H), 7.52-7.58 (m, 2H), 7.69 (d, 1H), 8.66 (m, 1H), 8.78 (dd, 1H), 9.04 (s, 1H), 9.69 (br, NH).

### EXAMPLE 56C

### N-Cyclopropyl-1-[3-(4-methylsulfonylmethylphenyl)phenyl]-1,4-dihydro[1,8]naphthyridin-4-one-3-carboxamide

### Step 1: N-Cyclopropyl-1-[3-(4-bromomethylphenyl)phenyl]-1,4-dihydro[1,8]naphthyridin-4-one-3-carboxamide

A mixture of N-cyclopropyl-1-[3-(4-hydroxymethylphenyl)phenyl]-1,4-dihydro[1,8]naphthyridin-4-one-3-carboxamide from Example 16C, carbon tetrabromide (2 eq), and diphos (0.6 molareq) in methylene chloride (15 ml/mmol) was stirred at room temperature for 3 hours. The mixture was concentrated at room temperature and chromatographed on silica gel eluting with a 1:1 mixture of ethyl acetate and methylene chloride to afford the N-Cyclopropyl-1-[3-(4-bromomethylphenyl)phenyl]-1,4-dihydro[1,8]naphthyridin-4-one-3-carboxamide compound.

### Step 2: N-Cyclopropyl-1-[3-(4-methylsulfonylmethylphenyl)phenyl]-1,4-dihydro[1,8]naphthyridin-4-one-3-carboxamide

To a solution of N-Cyclopropyl-1-[3-(4-bromomethylphenyl)phenyl]-1,4-dihydro[1,8]naphthyridin-4-one-3-carboxamide from Step 1 in N,N-dimethylformamide (20 ml/mmol) was added methanesulfinic acid sodium salt (1.3 eq) and the resulting mixture was stirred at room temperature for 18 hours. To the mixture was added saturated aqueous ammonium chloride solution and ethyl acetate, and the insoluble solid was filtered and washed well with water, hexane, ether and ethyl acetate to afford the title compound as a solid.

¹H NMR (CDCl₃) δ 0.72 (m, 2H), 0.89 (m, 2H), 2.85 (s, 3H), 3.04 (m, 1H), 4.34 (s, 2H) 7.46-7.52 (m, 2H), 7.55 (d, 2H), 7.65-7.73 (m, 4H), 7.80 (d, 1H), 8.76 (m, 1H), 8.85 (d, 1H), 9.12 (s, 1H), 9.82 (br, NH).

### EXAMPLE 57

### N-Cyclopropyl-1-[3-(1,6-dihydro-6-oxopyridin-3-yl)phenyl]-1,4-dihydro[1,8]naphthyridin-4-one-3-carboxamide

To a solution of N-cyclopropyl-1-[3-(6-benzyloxypyridin-3-yl)phenyl]-1,4-dihydro[1,8]naphthyridin-4-one-3-carboxamide from Example 42C in 1,2-dichloroethane (25 ml/mmol) was added trifluoroacetic acid (1.5 ml/mmol) and the resulting mixture was stirred at 60° C. for 18 hours. More trifluoroacetic acid was added (0.75 ml/mmol) and heating was continued for a further 24 hours. The cooled mixture was diluted with methylene chloride and saturated aqueous sodium bicarbonate was added, resulting in precipitation of a solid which was filtered. From the filtrate the organic phase was collected and evaporated to a solid which was combined with the previous filtered solid. This mixture was chromatographed on silica gel eluting with 10% methanol in methylene chloride to afford the title compound as a white fluffy solid.

¹H NMR (DMSO-d₆) δ 0.57 (m, 2H), 0.78 (m, 2H), 2.90 (m, 1H), 6.45 (d, 1H), 7.52 (m, 1H), 7.61-7.65 (m, 2H), 7.78 (d, 1H), 7.85 (s, 1H), 7.89-7.93 (m, 2H), 8.74 (d, 1H), 8.78-8.81 (m, 2H), 9.73 (br, NH), other NH>11 ppm.

### EXAMPLE 58C

### N-Cyclopropyl-1-[[3-{5-[6-(1-hydroxy-1-methylethyl)pyridin-3-yl]pyridin-3-yl}phenyl]]-1,4-dihydro[1,8]naphthyridin-4-one-3-carboxamide

Following the procedure of Step 4 of Example 47C, but substituting N-cyclopropyl-1-[3-(5-bromopyridin-3-yl)phenyl]-1,4-dihydro[1,8]naphthyridin-4-one-3-carboxamide from Example 41C for N-cyclopropyl-1-(3,5-dibromophenyl)-1,4-dihydro[1,8]naphthyridin-4-one-3-carboxamide, the title compound was obtained as a white solid.

¹H NMR (DMSO-d₆) δ 0.58 (m, 2H), 0.79 (m, 2H), 2.91 (m, 1H), 5.30 (s, 1H, OH), 7.65 (m, 1H), 7.71-7.79 (m, 3H), 8.12 (d, 1H), 8.23-8.26 (m, 2H), 8.49 (s, 1H), 8.75 (dd, 1H), 8.80 (m, 1H), 8.87 (s, 1H), 8.97 (m, 2H), 9.04 (s, 1H), 9.74 (br, NH).

### EXAMPLE 59C

### N-Isopropyl-1-[3-(1-oxidopyridin-3-yl)phenyl]-1,4-dihydro[1,8]naphthyridin-4-one-3-carboxamide

Following the procedure of Step 2 of Example 30, but substituting N-isopropyl-1-[3-(pyridin-3-yl)phenyl]-1,4-dihydro[1,8]naphthyridin-4-one-3-carboxamide from Example 7C for 5-bromo-2-(1-hydroxy-1-methylethyl)pyridine, the title compound was obtained as a white solid.

¹H NMR (DMSO-d₆) δ 1.21 (d, 6H), 4.10 (m, 1H), 7.51 (t, 1H), 7.64 (m, 1H), 7.71-7.75 (m, 3H), 7.97 (m, 1H), 8.09 (s, 1H), 8.23 (d, 1H), 8.69-8.77 (m, 3H), 8.84 (s, 1H), 9.66 (br, NH).

### EXAMPLE 60C

### N-(2,6-Dichloropyridin-4-yl)-1-[3-(1-oxidopyridin-3-yl)phenyl]-1,4-dihydro[1,8]naphthyridin-4-one-3-carboxamide

Following the procedure of Step 2 of Example 30C, but substituting N-(2,6-dichloropyridin-4-yl)-1-[3-(pyridin-3-yl)phenyl]-1,4-dihydro[1,8]naphthyridin-4-one-3-carboxamide from Example 10 for 5-bromo-2-(1-hydroxy-1-methylethyl)pyridine, the title compound was obtained as a white solid.

¹H NMR (DMSO-d₆) δ 7.51 (m, 1H), 7.69-7.78 (m, 4H), 7.99 (dd, 1H), 8.14 (s, 1H), 8.24 (dd, 1H), 8.70 (s, 1H), 8.73 (s, 2H), 8.84 (m, 2H), 8.99 (s, 1H), 12.05 (br, NH).

### EXAMPLE 61C

### N-Isopropyl-1-[3-(5-carboethoxy-1-oxidopyridin-3-yl)phenyl]-1,4-dihydro[1,8]naphthyridin-4-one-3-carboxamide

Following the procedure of Step 2 of Example 30C, but substituting N-isopropyl-1-[3-(5-carboethoxypyridin-3-yl)phenyl]-1,4-dihydro[1,8]naphthyridin-4-one-3-carboxamide from Example 24C for 5-bromo-2-(1-hydroxy-1-methylethyl)pyridine, the title compound was obtained as a white solid.

¹H NMR (CDCl₃) δ 1.28 (d, 6H), 1.40 (t, 3H), 4.28 (m, 1H), 4.43 (q, 2H), 7.49 (dd, 1H), 7.56 (m, 1H), 7.68 (s, 1H), 7.73 (d, 2H), 8.04 (s, 1H), 8.60 (s, 1H), 8.68 (dd, 1H), 8.77 (s, 1H), 8.82 (d, 1H), 9.01 (s, 1H), 9.61 (br, NH).

### EXAMPLE 62C

### N-Isopropyl-1-{3-[5-(1-hydroxy-1-methylethyl)-1-oxidopyridin-3-yl]phenyl}-1,4-dihydro[1,8]naphthyridin-4-one-3-carboxamide

Following the procedure of Step 2 of Example 30C, but substituting N-isopropyl-1-{3-[5-(1-hydroxy-1-methylethyl)pyridin-3-yl]phenyl}-1,4-dihydro[1,8]naphthyridin-4-one-3-carboxamide from Example 25C for 5-bromo-2-(1-hydroxy-1-methylethyl)pyridine, the title compound was obtained as a white solid

¹H NMR (CDCl₃) δ 1.29 (d, 6H), 1.60 (s, 6H), 4.11 (brs, 1H), 4.23 (m, 1H), 7.42-7.51 (m, 2H), 7.58 (s, 2H) 7.65 (m, 2H), 8.28 (s, 1H), 8.33 (s, 1H), 8.64 (m, 1H), 8.80 (d, 1H), 8.98 (s, 1H), 9.61 (br, NH).

### EXAMPLE 63C

### N-Isopropyl-1-{3-[6-(2-methylpropyl)-1-oxidopyridin-3-yl]phenyl-1,4-dihydro [1,8]naphthyridin-4-one-3-carboxamide

Following the procedure of Step 2 of Example 30, but substituting N-isopropyl-1-{3-[6-(2-methylpropyl)pyridin-3-yl]phenyl}-1,4-dihydro[1,8]naphthyridin-4-one-3-carboxamide from Example 26C for 5-bromo-2-(1-hydroxy-1-methylethyl)pyridine, the title compound was obtained as an off-white solid.

¹H NMR (CDCl₃) δ 0.98 (d, 6H), 1.29 (d, 6H), 2.29 (m, 1H), 2.32 (d, 2H), 4.26 (m, 1H), 7.28 (d, 1H), 7.38 (d, 1H), 7.47-7.52 (m, 2H), 7.60 (s, 1H), 7.69 (m, 2H), 8.53 (s, 1H), 8.69 (m, 1H), 8.82 (dd, 1H), 9.03 (s, 1H), 9.62 (br, NH).

### EXAMPLE 64C

### N-Isopropyl-1-[3-(6-methyl-1-oxidopyridin-3-yl)phenyl]-1,4-dihydro[1,8]naphthyridin-4-one-3-carboxamide

Following the procedure of Step 2 of Example 30, but substituting N-isopropyl-1-[3-(6-methylpyridin-3-yl)phenyl]-1,4-dihydro[1,8]naphthyridin-4-one-3-carboxamide from Example 28C for 5-bromo-2-(1-hydroxy-1-methylethyl)pyridine, the title compound was obtained as an off-white solid.

¹H NMR (CDCl₃) δ 1.32 (d, 6H), 2.60 (s, 3H), 4.30 (m, 1H), 7.35-7.45 (m, 2H), 7.50 (m, 2H), 7.62 (s, 1H), 7.72 (d, 2H), 8.58 (s, 1H), 8.72 (m, 1H), 8.85 (dd, 1H), 9.06 (s, 1H), 9.66 (br, NH).

### EXAMPLE 65C

### N-Cyclopropyl-1-[3-(1-oxidopyridin-3-yl)phenyl]-1,4-dihydro[1,8]naphthyridin-4-one-3-carboxamide

Following the procedure of Step 2 of Example 30C, but substituting N-cyclopropyl-1-[3-(pyridin-3-yl)phenyl]-1,4-dihydro[1,8]naphthyridin-4-one-3-carboxamide from Example 14C for 5-bromo-2-(1-hydroxy-1-methylethyl)pyridine, the title compound was obtained as a white solid.

¹H NMR (DMSO-d₆) δ 0.57 (m, 2H), 0.78 (m, 2H), 2.90 (m, 1H), 7.52 (t, 1H), 7.65 (m, 1H), 7.72-7.76 (m, 3H), 7.98 (m, 1H), 8.10 (s, 1H), 8.25 (d, 1H), 8.70-8.79 (m, 3H), 8.85 (s, 1H), 9.72 (br, NH).

### EXAMPLE 66C

### N-Cyclopropyl-1-{3-[6-(1-hydroxy-1-methylethyl)-1-oxidopyridin-3-yl]phenyl}-1,4-dihydro[1,8]naphthyridin-4-one-3-carboxamide

Following the procedure of Step 2 of Example 29C but substituting 5-bromo-2-(1-hydroxy-1-methylethyl)pyridine N-oxide from Step 2 of Example 30C for 5-bromo-1-oxidopyrimidine, the title compound was obtained as a white solid.

¹H NMR (CDCl₃) δ 0.66 (m, 2H), 0.85 (m, 2H), 1.70 (s, 6H), 2.97 (m, 1H), 7.43-7.49 (m, 2H), 7.52-7.56 (m, 2H), 7.61 (s, 2H) 7.71-7.74 (m, 2H), 8.49 (s, 1H), 8.68 (m, 1H), 8.80 (d, 1H), 9.02 (s, 1H), 9.74 (br, NH).

### EXAMPLE 67C

### N-Cyclopropyl-1-[3-(1-oxidopyridin-4-yl)phenyl]-1,4-dihydro[1,8]naphthyridin-4-one-3-carboxamide

Following the procedure of Step 2 of Example 30C, but substituting N-cyclopropyl-1-[3-(pyridin-4-yl)phenyl]-1,4-dihydro[1,8]naphthyridin-4-one-3-carboxamide from Example 17C for 5-bromo-2-(1-hydroxy-1-methylethyl)pyridine, the title compound was obtained as a white solid.

¹H NMR (DMSO-d,) δ 0.57 (m, 2H), 0.79 (m, 2H), 2.92 (m, 1H), 7.62-7.70 (m, 2H), 7.75 (t, 1H), 7.88 (d, 2H), 8.03 (d, 1H), 8.15 (s, 1H), 8.30 (d, 2H), 8.75 (d, 1H), 8.80 (m, 1H), 8.86 (s, 1H), 9.73 (br, NH).

### EXAMPLE 68C

### N-Cyclopropyl-1-[3-(5-bromo-1-oxidopyridin-3-yl)phenyl]-1,4-dihydro[1,8]naphthyridin-4-one-3-carboxamide

Following the procedure of Step 2 of Example 30, but substituting N-cyclopropyl-1-[3-(5-bromopyridin-3-yl)phenyl]-1,4-dihydro[1,8]naphthyridin-4-one-3-carboxamide from Example 41C for 5-bromo-2-(1-hydroxy-1-methylethyl)pyridine, the title compound was obtained as a light yellow solid.

¹H NMR (DMSO-d₆) δ 0.56 (m, 2H), 0.78 (m, 2H), 2.91 (m, 1H), 7.65 (m, 1H), 7.71-7.74 (m, 2H), 8.02-8.06 (m, 2H), 8.15 (s, 1H), 8.60 (s, 1H), 8.73-8.79 (m, 3H), 8.86 (s, 1H), 9.73 (br, NH).

### EXAMPLE 69C

### N-Cyclopropyl-1-[[3-{5-[6-(1-hydroxy-1-methylethyl)-1-oxidopyridin-3-yl]pyridin-3-yl}phenyl]]-1,4-dihydro[1,8]naphthyridin-4-one-3-carboxamide

Following the procedure of Step 2 of Example 30C, but substituting N-cyclopropyl-1-[[3-{5-[6-(1-hydroxy-1-methylethyl)pyridin-3-yl]pyridin-3-yl}phenyl]]-1,4-dihydro[1,8]naphthyridin-4-one-3-carboxamide from Example 58 for 5-bromo-2-(1-hydroxy-1-methylethyl)pyridine, and using 1.6 eq. of m-chloroperoxybenzoic acid, the title compound was obtained as a white solid.

¹H NMR (DMSO-d₆) δ 0.57 (m, 2H), 0.78 (m, 2H), 2.91 (m, 1H), 6.94 (s, 1H, OH), 7.65 (m, 1H), 7.71-7.79 (m, 3H), 7.97 (dd, 1H), 8.13 (d, 1H), 8.25 (s, 1H), 8.55 (s, 1H), 8.74 (dd, 1H), 8.80 (m, 1H), 8.87 (s, 1H), 8.91 (s, 1H), 9.00 (s, 1H), 9.09 (s, 1H), 9.73 (br, NH).

### EXAMPLE 70C

### N-Cyclopropyl-1-[[3-{5-[6-(1-hydroxy-1-methylethyl)pyridin-3-yl]-1-oxidopyridin-3-yl}phenyl]]-1,4-dihydro[1,8]naphthyridin-4-one-3-carboxamide

From the procedure of Example 69C, the title compound was also obtained as a white solid.

¹H NMR (DMSO-d₆) δ 0.57 (m, 2H), 0.79 (m, 2H), 2.92 (m, 1H), 5.32 (s, 1H, OH), 7.65 (m, 1H), 7.72-7.80 (m, 3H), 8.08-8.17 (m, 2H), 8.27 (m, 2H), 8.70-8.82 (m, 4H), 8.88 (s, 1H), 8.98 (s, 1H), 9.73 (br, NH).

### EXAMPLE 71C

### N-Cyclopropyl-1-[[3-{5-[6-(1-hydroxy-1-methylethyl)-1-oxidopyridin-3-yl]-1-oxidopyridin-3-yl}phenyl]]-1,4-dihydro[1,8]naphthyridin-4-one-3-carboxamide

From the procedure of Example 69C the title compound was also obtained as a white solid.

¹H NMR (DMSO-d₆) δ 0.58 (m, 2H), 0.80 (m, 2H), 2.92 (m, 1H), 6.85 (brs, 1H, OH), 7.65 (m, 1H), 7.70-7.80 (m, 3H), 7.96 (d, 1H), 8.13 (m, 2H), 8.29 (s, 1H), 8.71-8.84 (m, 4H), 8.89 (s, 1H), 8.92 (s, 1H), 9.73 (br, NH).

### EXAMPLE 72C

### N-Isopropyl-1-[3-(1-oxidoquinolin-3-yl)phenyl]-1,4-dihydro[1,8]naphthyridin-4-one-carboxamide

Following the procedure of Step 2 of Example 30, but substituting N-isopropyl-1-[3-(quinolin-3-yl)phenyl]-1,4-dihydro[1,8]naphthyridin-4-one-carboxamide from Example 12C for 5-bromo-2-(1-hydroxy-1-methylethyl)pyridine, the title compound was obtained as a solid.

¹H NMR (CDCl₃) δ 1.30 (d, 6H), 4.28 (m, 1H), 7.49 (dd, 1H), 7.54 (d, 1H), 7.66-7.85 (m, 5H), 7.92 (m, 2H), 8.69-8.75 (m, 2H), 8.84 (d, 1H), 8.86 (s, 1H), 9.08 (s, 1H), 9.64 (br, NH).

### EXAMPLE 73C

### N-Isobutyl-1-{3-[6-(1-hydroxy-1-methylethyl)-1-oxidopyridin-3-yl]phenyl}-1,4-dihydro [1,8]naphthyridin-4-one-3-carboxamide

To a mixture of N-isobutyl-1-{3-[6-(1-hydroxy-1-methylethyl)pyridin-3-yl]phenyl}-1,4-dihydro[1,8]naphthyridin-4-one-3-carboxamide from Example 46 in 13:1 methylene chloride/methanol (33 ml/mmol) at room temperature was added magnesium monoperoxyphthalate hexahydrate (MMPP, 1.1 molareq) and the resulting mixture was stirred at room temperature for 24 hours. The mixture was filtered through a bed of celite and the filtrate was washed with aqueous sodium carbonate, then water and dried. The crude product was chromatographed on silica gel eluting with 8% ethanol in ethyl acetate and the solid obtained was stirred at room temperature in ether for several hours and filtered to afford the title compound as a light pink solid.

¹H NMR (Acetone-d₆) δ 0.98 (d, 6H), 1.61 (s, 6H), 1.88 (m, 1H), 3.26 (t, 2H), 7.52 (s, 1H, OH), 7.61 (m, 1H), 7.66 (d, 1H), 7.77-7.82 (m, 2H), 7.88 (d, 1H), 7.99 (d, 1H), 8.12 (s, 1H), 8.68 (s, 1H), 8.73 (m, 1H), 8.80 (dd, 1H), 8.93 (s, 1H), 9.81 (br, NH).

### EXAMPLE 74C

### N-Cyclopropyl-1-[3-(6-methyl-1-oxidopyridin-3-yl)]phenyl]-1,4-dihydro[1,8]naphthyridin-4-one-3-carboxamide

Following the procedure of Example 73, but substituting N-cyclopropyl-1-[3-(6-methylpyridin-3-yl)]phenyl]-1,4-dihydro[1,8]naphthyridin-4-one-3-carboxamide from Example 39C for N-isobutyl-1{[3-[6-(1-hydroxy-1-methylethyl)pyridin-3-yl]phenyl}-1,4-dihydro[1,8]naphthyridin-4-one-3-carboxamide, the title compound was obtained as a solid.

¹H NMR (CDCl₃) δ 0.72 (m, 2H), 0.91 (m, 2H), 2.61 (s, 3H), 3.02 (m, 1H), 7.38 (d, 1H), 7.45 (dd, 1H), 7.49-7.58 (m, 2H), 7.66 (s, 1H), 7.75 (m, 2H), 8.61 (s, 1H), 8.72 (m, 1H), 8.87 (dd, 1H), 9.08 (s, 1H), 9.78 (br, NH).

### EXAMPLE 75C

### N-Cyclopropyl-1-[3-(6-methylsulfonyl-1 oxidopyridin-3-yl)phenyl]-1,4-dihydro [1,8]naphthyridin-4-one-3-carboxamide

To a suspension of N-cyclopropyl-1-[3-(6-methylsulfonylpyridin-3-yl)phenyl]-1,4-dihydro[1,8]naphthyridin-4-one-3-carboxamide from Example 51 in methylene chloride (30 ml/mmol) was added urea-hydrogen peroxide (8 eq) and the resulting mixture was cooled to 0° C. Trifluoroacetic acid (4.7 eq) was added and the mixture was warmed to room temperature as a solution was obtained. After 18 hours, more urea-hydrogen peroxide (2.6 eq) and trifluoroacetic acid (2 eq) were added and stirring was continued for 2 hours. The mixture was quenched with saturated aqueous sodium metabisulfite, diluted with methylene chloride and the organic phase was washed with 1N aqueous HCl, then brine and water, dried and evaporated. The crude product was chromatographed on silica gel eluting with 40% toluene in acetone to afford the title compound as a solid.

¹H NMR (CDCl₃) δ 0.66 (m, 2H), 0.85 (m, 2H), 2.97 (m, 1H), 3.52 (s, 3H), 7.48 (m, 1H), 7.58-7.65 (m, 3H), 7.72-7-78 (m, 2H), 8.15 (d, 1H), 8.54 (s, 1H), 8.68 (brs, 1H), 8.81 (d, 1H), 9.01 (s, 1H), 9.71 (br, NH).

### EXAMPLE 76C

### N-Cyclopropyl-1-{5-bromo-3-[6-(1-hydroxy-1-methylethyl)-1-oxidopyridin-3-yl]phenyl}-1,4-dihydro[1,8]naphthyridin-4-one-3-carboxamide

Following the procedure of Step 2 of Example 30C, but substituting N-cyclopropyl-1-{5-bromo-3-[6-(1-hydroxy-1-methylethyl)pyridin-3-yl]phenyl}-1,4-dihydro[1,8]naphthyridin-4-one-3-carboxamide from Example 47C for 5-bromo-2-(1-hydroxy-1-methylethyl)pyridine, the title compound was obtained as a solid.

¹H NMR (CDCl₃) δ 0.71 (m, 2H), 0.90 (m, 2H), 1.75 (s, 6H), 3.02 (m, 1H), 7.48-7.60 (m, 5H), 7.73 (s, 1H), 7.88 (s, 1H), 8.52 (s, 1H), 8.72 (m, 1H), 8.84 (dd, 1H), 9.04 (s, 1H), 9.71 (br, NH).

### EXAMPLE 77C

### N-Cyclopropyl-1-{3-[6-(1,2-dihydroxy-1-methylethyl)-1-oxidopyridin-3-yl]phenyl}-1,4-dihydro[1,8]naphthyridin-4-one-3-carboxamide

### Step 1: 5-Bromo-2-(1-methylvinyl)pyridine N-oxide

A mixture of 5-bromo-2-(1-hydroxy-1-methylethyl)pyridine N-oxide from step 2 of example 30 (1.29 g) and 25% aqueous sulfuric acid was heated at 130° C. for 2 days. After cooling, the mixture was made slightly basic using 10N aqueous sodium hydroxide and partitioned between ethyl acetate and water. The crude product from evaporation of the organic phase was used as such in step 2.

### Step 2: 5-bromo-2-(1,2-dihydroxy-1-methylethyl)pyridine N-oxide

The crude product from step 1 was dissolved in a 3:1 mixture of acetone and water (16 mL) and 4-methylmorpholine N-oxide (1 g) and potassium osmate dihydrate (90 mg) were added. The resulting mixture was stirred at room temperature for 3 days then excess solid sodium bisulfite was added and the mixture was evaporated. The residue was diluted with methylene chloride and filtered. The filtrate was evaporated and the residue chromatographed on silica gel eluting with ethyl acetate to afford the title compound as a white solid.

### Step 3: N-Cyclopropyl-1-{3-[6-(1,2-dihydroxy-1-methylethyl)-1-oxidopyridin-3-yl]phenyl}-1,4-dihydro[1,8)naphthyridin-4-one-3-carboxamide

Following the procedure of step 2 of example 32 but substituting 5-bromo-2-(1,2-dihydroxy-1-methylethyl)pyridine N-oxide from step 2 for 3-bromo-5-methylsulfonylpyridine the title compound was obtained as a white solid.

¹H NMR (CDCl₃) δ 0.66 (m, 2H), 0.85 (m, 2H), 1.61 (s, 3H), 2.78 (m, 1H, OH), 2.97 (m, 1H), 3.90 (m, 1H), 3.97 (m, 1H), 7.48 (m, 1H), 7.53 (m, 2H), 7.60 (m, 2H), 7.69-7.72 (m, 2H), 7.92 (s, 1H, OH), 8.49 (s, 1H), 8.68 (m, 1H), 8.80 (dd, 1H), 9.02 (s, 1H), 9.73 (br, NH).

Referring to the formula below, Examples 1D-33D are summarized in TABLE 1D below.

TABLE 1

| | | | | |
|---|---|---|---|---|
| Ex. | R¹ | R⁸R²R³ | R⁴ | R⁶ |

| | | | | |
|---|---|---|---|---|
| 1D | i-pr | Ph | H | H |
| 2D | i-pr | 2-Pyr | H | H |
| 3D | i-pr | 4-Pyr | H | H |
| 4D | i-pr | 4-PyrNO | H | H |
| 5D | i-pr | H | H | H |
| 6D | c-pr | H | H | H |
| 7D | i-pr | 3-Pyr | H | H |
| 8D | i-pr | 3-PyrNO | H | H |
| 9D | c-pr | 3-Pyr | H | H |
| 10D | i-pr | 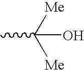 | H | H |
| 11D | c-pr | 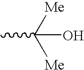 | H | H |
| 12D | i-pr | 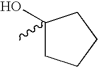 | H | H |
| 13D | i-pr | 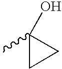 | H | H |
| 14D | i-pr | 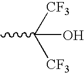 | H | H |
| 15D | i-pr | 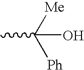 | H | H |
| 16D | c-pr | 3-PyrNO | H | H |
| 17D | i-pr | 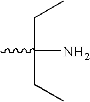 | H | H |
| 18D | c-pr | 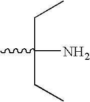 | H | H |
| 19D | i-pr | 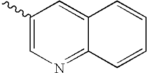 | H | H |
| 20D | i-pr | 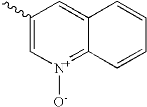 | H | H |
| 21D | i-pr | 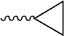 | H | H |
| 22D | i-pr | 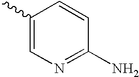 | H | H |
| 23D | i-pr | 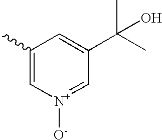 | H | H |
| 24D | i-pr | 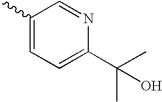 | H | H |
| 25D | i-pr | 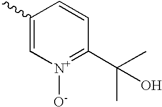 | H | H |
| 26D | i-pr | 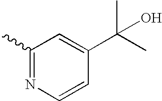 | H | H |
| 27D | i-pr | 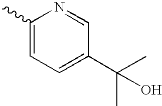 | H | H |
| 28D | i-pr | 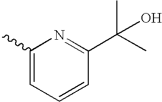 | H | H |
| 29D | c-pr | 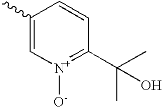 | H | H |
| 30D | i-pr | 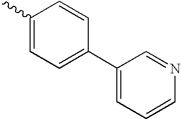 | H | H |
| 31D | i-pr | 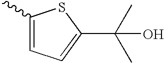 | H | H |
| 32D | i-pr | 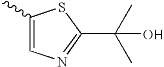 | H | H |
| 33D | H | 3-PyrNO | H | H |

### EXAMPLE 1D

### N-Isopropyl-1-[3-(phenylethynyl)phenyl]-1,4-dihydro[1,8]naphthyridin-4-one-3-carboxamide

### Step 1: Ethyl 3-(3-bromoanilino)-2-(2-chloronicotinoyl) acrylate

A mixture of ethyl 2-chloronicotinoyl acetate (41.1 g, 180.5 mmol), triethyl orthoformate (40.12 g, 271 mmol) and acetic anhydride (92.05 g, 902.5 mmol) was heated at 130° C. for 2.5 hours. The volatile components were distilled off and the resulting residue was co-evaporated twice with xylene. The oily residue was dissolved in methylene chloride (250 mL) and 3-bromoaniline (37.25 g, 216.6 mmol) was added slowly. The resulting solution was stirred at room temperature for 18 hours, and the solvent evaporated away. The resulting crude compound was used as such in the next step.

### Step 2: Ethyl 1-(3-bromophenyl)-1,4-dihydro[1,8]naphthyridin-4-one-3-carboxylate

The crude compound from Step 1 was dissolved in tetrahydrofuran (500 mL), the solution was cooled to 0° C., and sodium hydride (as a 60% dispersion in oil, 9.4 g, 235 mmol) was added in portions. After stirring at 0° for 1 hour, the resulting mixture was allowed to warn up to room temperature. After 2 hours, water (400 mL) was added to the resulting suspension and the insoluble solid was filtered and washed copiously with water. When dry, the solid was stirred in ether (150 mL) at room temperature for 24 hours and filtered to afford the ethyl 1-(3-bromophenyl)-1,4-dihydro[1,8]naphthyridin-4-one-3-carboxylate compound as a cream-colored solid.

¹H NMR (Acetone-d₆) δ 1.32 (t, 3H), 4.29 (q, 2H), 7.54-7.63 (m, 2H), 7.69 (dd, 1H), 7.78 (dd, 1H), 7.93 (s, 1H), 8.66-8.71 (m, 3H).

### Step 3: 1-(3-Bromophenyl)-1,4-dihydro[1,8]naphthyridin-4-one-3-carboxylic acid

A suspension of ethyl 1-(3-bromophenyl)-1,4-dihydro[1,8]naphthyridin-4-one-3-carboxylate from Step 2 (52.5 g, 140.7 mmol) in a mixture of tetrahydrofuran (400 mL), methanol (400 mL) and 1N aqueous sodium hydroxide (280 mL) was heated at ca 50° C. with stirring for 20 minutes. After cooling, the mixture was diluted with water (300 mL) and 1N aqueous HCl (325 mL) was added. After stirring for 45 minutes, the precipitate was filtered, washed well with water and dried to afford the 1-(3-bromophenyl)-1,4-dihydro[1,8]naphthyridin-4-one-3-carboxylic acid as a cream-colored solid.

¹H NMR (Acetone-d₆) δ 7.65 (t, 1H), 7.76 (m, 2H), 7.84 (d, 1H), 7.99 (s, 1H), 8.87 (m, 2H), 9.01 (s, 1H).

### Step 4: N-Isopropyl-1-(3-bromophenyl)-1,4-dihydro[1,8]naphthyridin-4-one-3-carboxamide

To a suspension of 1-(3-bromophenyl)-1,4-dihydro[1,8]naphthyridin-4-one-3-carboxylic acid from Step 3 (26.3 g, 76 mmol) and triethylamine (23.2 g, 230 mmol) in tetrahydrofuran (100 mL) at 0° C. was added isobutyl chloroformate (18.85 g, 138 mmol). After stirring at 0° C. for 2 hours, isopropylamine (23 g, 390 mmol) was added and the mixture was allowed to warm up to room temperature and stirred overnight. The mixture was then partitioned between ethyl acetate and water, the organic phase was dried and evaporated to a solid which was stirred in ether at room temperature for 3 hours and filtered to afford the N-isopropyl-1-(3-bromophenyl)-1,4-dihydro[1,8]naphthyridin-4-one-3-carboxamide as a white solid.

¹H NMR (Acetone-d₆) δ 1.25 (d, 6H), 4.17 (m, 1H), 7.59-7.63 (m, 2H), 7.70 (d, 1H), 7.80 (d, 1H), 7.94 (s, 1H), 8.73 (m, 1H), 8.78 (d, 1H), 8.85 (s, 1H), 9.61 (br, NH).

### Step 5: N-Isopropyl-1-[(3-phenylethynyl)phenyl]-1,4-dihydro[1,8]naphtherdin-4-one-3-carboxamide

A mixture of amide from Step 4, phenylacetylene (1.9 eq), triethylamine (1.6 eq), triphenylphosphine (0.06 eq) and bis(triphenylphosphine)palladium (II) chloride (0.05 eq) in THF (16 mL/mmol) was stirred at room temperature for 20 minutes. Copper (I) iodide (5 mg/mmol) was added and the mixture was stirred at reflux for 18 hours. After cooling, the mixture was quenched with saturated aqueous ammonium chloride solution and partitioned between ethyl acetate and water. The organic phase was dried over magnesium sulfate and the crude product was chromatographed on silica gel eluting with a 1:9 mixture of ether and methylene chloride to afford a solid which was stirred in ether at room temperature and filtered to yield the N-Isopropyl-1-[(3-phenylethynyl)phenyl]-1,4-dihydro[1,8]naphthyridin-4-one-3-carboxamide compound as a solid.

¹H NMR (Acetone-d₆) δ 1.24 (d, 6H), 4.18 (m, 1H), 7.42 (m, 3H), 7.56-7.61 (m, 3H), 7.69 (m, 2H), 7.76 (m, 1H), 7.85 (s, 1H), 8.73 (m, 1H), 8.77 (dd, 1H), 8.88 (s, 1H), 9.62 (br, NH).

### EXAMPLE 2D

### N-Isopropyl-1-[3-(2-pyridinylethynyl)phenyl]-1,4-dihydro[1,8]naphthyridin-4-one-3-carboxamide

Following the procedure of Step 5 of EXAMPLE 1D, but substituting 2-ethynylpyridine for phenylacetylene, the title compound was obtained as a brown solid.

¹H NMR (Acetone-d₆) δ 1.25 (d, 6H), 4.18 (m, 1H), 7.38 (m, 1H), 7.59-7.64 (m, 2H), 7.71-7.76 (m, 2H), 7.81-7.85 (m, 2H), 7.92 (s, 1H), 8.61 (m, 1H), 8.74 (m, 1H), 8.78 (dd, 1H), 8.89 (s, 1H), 9.62 (br, NH).

### EXAMPLE 3D

### N-Isopropyl-1-[3-(4-pyridinylethynyl)phenyl]-1,4-dihydro[1,8]naphthyridin-4-one-3-carboxamide

Following the procedure of Step 5 of EXAMPLE 1D, but substituting 4-ethynylpyridine (J. Org. Chem. 1996, 61, 6535) for phenylacetylene, the title compound was obtained as a solid.

¹H NMR (Acetone-d₆) δ 1.24 (d, 6H), 4.18 (m, 1H), 7.49 (m, 2H), 7.61 (m, 1H), 7.71-7.78 (m, 2H), 7.81 (m, 1H), 7.92 (s, 1H), 8.62 (m, 2H), 8.73 (m, 1H), 8.78 (dd, 1H), 8.87 (s, 1), 9.62 (br, NH).

### EXAMPLE 4D

### N-Isopropyl-1-[3-(1-oxido-4-pyridinylethynyl)phenyl]-1,4-dihydro[1,8]naphthyridin-4-one-3-carboxamide

To a solution of N-Isopropyl-1-[3-(4-pyridinylethynyl)phenyl]-1,4-dihydro[1,8]naphthyridin-4-one-3-carboxamide from EXAMPLE 3D in methylene chloride (36 mL/mmol) and methanol (3 mL/mmol) was added magnesium monoperoxyphthalate hexahydrate (MMPP, 3.6 eq) and the mixture was stirred at room temperature overnight. A further amount of MMPP (2 eq) was added and stirring was continued for 24 hours. The resulting mixture was filtered through a bed of celite, the filtrate was diluted with methylene chloride and washed with aqueous sodium bicarbonate and water. After drying, the organic phase was evaporated and the crude product was purified by chromatography on silica gel eluting with 10% methanol in methylene chloride to afford the title compound as a solid.

¹H NMR (CDCl₃) δ 1.28 (d, 6H), 4.28 (m, 1H), 7.35 (d, 2H), 7.46 (m, 2H), 7.58 (m, 2H), 7.67 (m, 1H), 8.14 (d, 2H), 8.69 (m, 1H), 8.81 (dd, 1H), 8.99 (s, 1H), 9.62 (br, NH).

### EXAMPLE 5D

### N-Isopropyl-1-[3-ethynylphenyl)-1,4-dihydro[1,8]naphthyridin-4-one-3-carboxamide

### Step 1: N-Isopropyl-1-[3-(trimethylsilylethynyl)phenyl]-1,4-dihydro[1,8]naphthyridin-4-one-3-carboxamide

Following the procedure of Step 5 of EXAMPLE 1D, but substituting trimethylsilylacetylene for phenylacetylene, the N-isopropyl-1-[3-(trimethylsilylethynyl)phenyl]-1,4-dihydro[1,8]naphthyridin-4-one-3-carboxamide product was obtained and used in the next step without further purification.

### Step 2: N-Isopropyl-1-(3-ethynylphenyl)-1,4-dihydro[1,8]naphthyridin-4-one-3-carboxamide

The crude product from Step 1 was dissolved in methanol (12 mL/mmol) and 1N aqueous sodium hydroxide was added (3 eq), resulting in a suspension. The suspension mixture was stirred at room temperature for 2 hours and the methanol was evaporated. The resulting aqueous suspension was diluted with water and the product was extracted out with ethyl acetate. The crude product was chromatographed on silica gel eluting with 10% ether in methylene chloride to afford the N-isopropyl-1-(3-ethynylphenyl)-1,4-dihydro[1,8]naphthyridin-4-one-3-carboxamide compound as a solid.

¹H NMR (Acetone-d₆) δ 1.24 (d, 6H), 3.81 (s, 1H), 4.17 (m, 1H), 7.59 (m, 1H), 7.64-7.71 (m, 3H), 7.81 (s, 1H), 8.72 (m, 1H), 8.76 (dd, 1H), 8.84 (s, 1H), 9.61 (br, NH).

### EXAMPLE 6D

### N-Cyclopropyl-1-(3-ethynylphenyl)-1,4-dihydro[1,8]naphthyridin-4-one-3-carboxamide

### Step 1: N-Cyclopropyl-1-(3-bromophenyl)-1,4-dihydro[1,8]naphthyridin-4-one-3-carboxamide

Following the procedure of Step 4 of EXAMPLE 1D, but substituting cyclopropylamine for isopropylamine, the N-cyclopropyl-1-(3-bromophenyl)-1,4-dihydro[1,8]naphthyridin-4-one-3-carboxamide was obtained as a fluffy white solid.

¹H NMR (Acetone-d₆) δ 0.59 (m, 2H), 0.80 (m, 2 h), 2.96 (m, 1H), 7.59-7.68 (m, 2H), 7.72 (dd, 1H), 7.82 (dd, 1H), 7.97 (s, 1H), 8.72-8.81 (m, 2H), 8.89 (s, 1H), 9.70 (br, NH).

### Steps 2 and 3: N-Cyclopropyl-1-(3-ethynylphenyl)-1,4-dihydro[1,8]naphthyridin-4-one-3-carboxamide

Following the procedures of Steps 1 and 2 of EXAMPLE 5D, but substituting the product from step 1 for N-isopropyl-1-(3-bromophenyl)-1,4-dihydro[1,8]naphthyridin-4-one-3-carboxamide, the N-Cyclopropyl-1-(3-ethynylphenyl)-1,4-dihydro[1,8]naphthyridin-4-one-3-carboxamide compound was obtained as a solid.

¹H NMR (CDCl₃) δ 0.66 (m, 2H), 0.85 (m, 2H), 2.97 (m, 1H), 3.18 (s, 1H), 7.42 (d, 1H), 7.47 (m, 1H), 7.52-7.58 (m, 2H), 7.65 (d, 1H), 8.70 (m, 1H), 8.80 (dd, 1H), 8.98 (s, 1H), 9.74 (br, NH).

### EXAMPLE 7D

### N-Isopropyl-1-[3-(3-pyridinylethynyl)phenyl]-1,4-dihydro[1,8]naphthyridin-4-one-3-carboxamide

Following the procedure of Step 5 of EXAMPLE 1D, but substituting N-isopropyl-1-(3-ethynylphenyl)-1,4-dihydro[1,8]naphthyridin-4-one-3-carboxamide from EXAMPLE 5 for phenylacetylene and 3-bromopyridine for N-isopropyl-1-(3-bromophenyl)-1,4-dihydro[1,8]naphthyridin-4-one-3-carboxamide, the title compound was obtained as a light brown solid.

¹H NMR (Acetone-d₆) δ 1.24 (d, 6H), 4.18 (m, 1H), 7.43 (m, 1H), 7.61 (m, 1H), 7.70-7.75 (m, 2H), 7.80 (d, 1H), 7.90 (s, 1H), 7.94 (d, 1H), 8.58 (m, 1H), 8.74-8.79 (m, 3H), 8.88 (s, 1H), 9.62 (br, NH).

### EXAMPLE 8D

### N-Isopropyl-1-[3-(1-oxido-3-pyridinylethynyl)phenyl]-1,4-dihydro[1,8]naphthyridin-4-one-3-carboxamide

Following the procedure of EXAMPLE 4D, but substituting N-isopropyl-1-[3-(3-pyridinylethynyl)phenyl]-1,4-dihydro[1,8]naphthyridin-4-one-3-carboxamide from EXAMPLE 7D for N-isopropyl-1-[3-(4-pyridinylethynyl)phenyl]-1,4-dihydro[1,8]naphthyridin-4-one-3-carboxamide, the title compound was obtained as a solid.

¹H NMR (CDCl₃) δ 1.28 (d, 6H), 4.28 (m, 1H), 7.26 (m, 1H), 7.36 (d, 1H), 7.45-7.49 (m, 2H), 7.57-7.62 (m, 2H), 7.69 (d, 1H), 8.16 (d, 1H), 8.31 (s, 1H), 8.69 (m, 1H), 8.81 (dd, 1H), 8.99 (s, 1H), 9.63 (br, NH).

### EXAMPLE 9D

### N-Cyclopropyl-1-[3-(3-pyridinylethynyl)phenyl]-1,4-dihydro[1,8]naphthyridin-4-one-3-carboxamide

Following the procedure of Step 5 of EXAMPLE 1D, but substituting N-cyclopropyl-1-(3-ethynylphenyl)-1,4-dihydro[1,8]naphthyridin-4-one-3-carboxamide from EXAMPLE 6 for phenylacetylene and 3-bromopyridine for N-isopropyl-1-(3-bromophenyl)-1,4-dihydro[1,8]naphthyridin-4-one-3-carboxamide, the title compound was obtained as a solid.

¹H NMR (CDCl₃) δ 0.66 (m, 2H), 0.85 (m, 2H), 2.97 (m, 1H), 7.28 (m, 1H), 7.43-7.48 (m, 2H), 7.57 (t, 1H), 7.62 (s, 1H), 7.70 (d, 1H), 7.79 (d, 1H), 8.55 (m, 1H), 8.70 (m, 1H), 8.75 (s, 1H), 8.79 (dd, 1H), 9.01 (s, 1H), 9.74 (br, NH).

### EXAMPLE 10D

### N-Isopropyl-1-[3-(3-hydroxy-3-methylbut-1-ynyl)phenyl]-1,4-dihydro[1,8]naphthyridin-4-one-3-carboxamide

Following the procedure of Step 5 of EXAMPLE 1D, but substituting 2-methyl-3-butyn-2-ol for phenylacetylene, the title compound was obtained as a white solid.

¹H NMR (Acetone-d₆) δ 1.24 (d, 6H), 1.53 (s, 6H), 4.17 (m, 1H), 4.52 (s, 1H, OH), 7.58-7.65 (m, 4H), 7.68 (s, 1H), 8.72 (m, 1H), 8.77 (dd, 1H), 8.84 (s, 1H), 9.62 (br, NH).

### EXAMPLE 11D

### N-Cyclopropyl-1-[3-(3-hydroxy-3-methylbut-1-ynyl)phenyl]-1,4-dihydro[1,8]naphthyridin-4-one-3-carboxamide

Following the procedure of EXAMPLE 10D, but substituting N-cyclopropyl-1-(3-ethynylphenyl)-1,4-dihydro[1,8]naphthyridin-4-one-3-carboxamide from EXAMPLE 6D for N-isopropyl-1-(3-ethynylphenyl)-1,4-dihydro[1,8]naphthyridin-4-one-3-carboxamide, the title compound was obtained as a white solid.

¹H NMR (Acetone-d₆) δ 0.57 (m, 2H), 0.78 (m, 2H), 1.53 (s, 6H), 2.93 (m, 1H), 4.53 (s, 1H, OH), 7.58-7.65 (m, 4H), 7.67 (s, 1H), 8.72 (m, 1H), 8.76 (dd, 1H), 8.85 (s, 1H), 9.69 (br, NH).

### EXAMPLE 12D

### N-Isopropyl-1-[3-(1-hydroxycyclopentyl)ethynylphenyl]-1,4-dihydro[1,8]naphthyridin-4-one-3-carboxamide

Following the procedure of Step 5 of EXAMPLE 1D, but substituting 1-ethynylcyclopentanol for phenylacetylene, the title compound was obtained as a white solid.

¹H NMR (CDCl₃) δ 1.28 (d, 6H), 1.76-1.80 (m, 2H), 1.84-1.88 (m, 3H), 1.98-2.06 (m, 4H), 4.27 (m, 1H), 7.36 (d, 1H), 7.44-7.50 (m, 3H), 7.56 (d, 1H), 8.68 (m, 1H), 8.79 (dd, 1H), 8.97 (s, 1H), 9.63 (br, NH).

### EXAMPLE 13D

### N-Isopropyl-1-[3-(1-hydroxycyclopropyl)ethynylphenyl]-1,4-dihydro[1,8]naphthyridin-4-one-3-carboxamide

### Step 1: 1-Ethynylcyclopropanol

The 1-ethynylcyclopropanol was prepared following the procedure described in J. Org. Chem. 1976, 41, 1237 from [(1-ethoxycyclopropyl)oxy]trimethylsilane and ethynyl magnesium bromide and was obtained as a liquid.

### Step 2: N-Isopropyl-1-[3-(1-hydroxcyclopropyl)ethynylphenyl]-1,4-dihydro[1,8]naphthyridin-4-one-3-carboxamide

Following the procedure of Step 5 of EXAMPLE 1D, but substituting the product from present Step 1 for phenylacetylene, the N-isopropyl-1-[3-(1-hydroxycyclopropyl)ethynylphenyl]-1,4-dihydro[1,8]naphthyridin-4-one-3-carboxamide compound was obtained as a solid.

¹H NMR (CDCl₃) δ 1.09 (m, 2H), 1.17 (m, 2H), 1.28 (d, 6H), 2.57 (s, 1H, OH), 4.28 (m, 1H), 7.35 (d, 1H), 7.44-7.50 (m, 3H), 7.54 (d, 1H), 8.68 (m, 1H), 8.79 (dd, 1H), 8.96 (s, 1H), 9.63 (br, NH).

### EXAMPLE 14D

### N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide

### Step 1: 1,1,1-trifluoro-2-(trifluoromethyl)-4-(trimethylsilyl)but-3-yn-2-ol

To a solution of trimethylsilylacetylene (4 mL) in TBF (30 mL) at −78° C. was added 2.5M n-butyllithium in hexanes (14 mL) and the resulting mixture was stirred for 1 hour. An excess of hexafluoroacetone was bubbled into the cold mixture and stirring was continued for 4 hours. After warming to room temperature, the mixture was quenched with saturated aqueous ammonium chloride solution and partitioned between ether and water. The organic phase was dried and evaporated to afford the 1,1,1-trifluoro-2-(trifluoromethyl)-4-(trimethylsilyl)but-3-yn-2-ol as a liquid.

### Step 2: N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide

To a solution of 1,1,1-trifluoro-2-(trifluoromethyl)-4-(trimethylsilyl)but-3-yn-2-ol from present Step 1 (6.8 mmol) in 10 nm of THF was added 1M tetrabutylammonium fluoride (8.5 mL) and the resulting mixture was refluxed for 30 minutes to remove the TMS protecting group. The procedure of Step 5 of EXAMPLE 1D was then applied, but substituting this solution for phenylacetylene to afford the N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide compound as a solid.

¹H NMR (Acetone-d₆) δ 1.24 (d, 6H), 4.17 (m, 1H), 7.60 (m, 1H), 7.72-7.79 (m, 2H), 7.83 (d, 1H), 7.90 (s, 1H), 8.14 (s, 1H, OH), 8.72 (m, 1H), 8.77 (dd, 1H), 8.85 (s, 1H), 9.62 (br, NH).

### EXAMPLE 15D

### N-Isopropyl-1-[3-(3-hydroxy-3-phenylbut-1-ynyl)phenyl]-1,4-dihydro[1,8]naphthyridin-4-one-3-carboxamide

A mixture of N-isopropyl-1-(3-bromophenyl)-1,4-dihydro[1,8]naphthyridin-4-one-3-carboxamide from Step 4 of EXAMPLE 1D, 2-phenyl-3-butyn-2-ol (2 eq), triethylamine (1.66 eq), bis(diphenylphosphino)ferrocene]dichloropalladium(H) (0.05 eq), and copper (I) iodide (5 mg/mmol) in DMF (20 mL/mmol) was heated at 85° C. for 18 hours. After cooling to room temperature, the resulting mixture was quenched with saturated aqueous ammonium chloride solution and partitioned between ethyl acetate and water. The crude product from the organic phase was chromatographed on silica gel eluting with 20% ether in methylene chloride. The purified product was stirred in ether at room temperature for 3 hours and filtered to afford the title compound as a white solid.

¹H NMR (Acetone-d₆) δ 1.24 (d, 6H), 1.79 (s, 3H), 4.18 (m, 1H), 5.22 (s, 1H, OH), 7.26 (t, 1H), 7.35 (t, 2H), 7.59 (m, 1H), 7.66 (m, 3H), 7.73 (d, 2H), 7.76 (s, 1H), 8.72 (m, 1H), 8.77 (dd, 1H), 8.84 (s, 1H), 9.62 (br, NH).

### EXAMPLE 16D

### N-Cyclopropyl-1-[3-(1-oxido-3-pyridinylethynyl)phenyl]-1,4-dihydro[1,8]naphthyridin-4-one-3-carboxamide

### Step 1: 3-Ethynylpyridine N-oxide

To a solution of 3-ethynylpyridine in methylene chloride (5 mL/mmol) at room temperature was added m-chloroperoxybenzoic acid (m-CPBA, 70% purity, 1.2 eq) and the resulting mixture was stirred for 2 hours. A further amount of m-CPBA was added (0.25 eq) and stirring was continued for 1 hour. Calcium hydroxide was added (2 eq) and after 15 minutes the mixture was filtered through celite and the filtrate was evaporated. The solid residue was stirred in ether for 3 hours and filtered to afford the 3-ethynylpyridine N-oxide compound as a white solid.

### Step 2: N-Cyclopropyl-1-[3-(1-oxido-3-pyridinylethynyl)phenyl]-1,4-dihydro[1,8]naphthyridin-4-one-3-carboxamide

Following the procedure of EXAMPLE 15D, but substituting N-cyclopropyl-1-(3-bromophenyl)-1,4-dihydro[1,8]naphthyridin-4-one-3-carboxamide from Step 1 of EXAMPLE 6D for N-isopropyl-1-(3-bromophenyl)-1,4-dihydro[1,8]naphthyridin-4-one-3-carboxamide, and 3-ethynylpyridine N-oxide from Step 1 for 2-phenyl-3-butyn-2-ol, the N-cyclopropyl-1-[3-(1-oxido-3-pyridinylethynyl)phenyl]-1,4-dihydro[1,8]naphthyridin-4-one-3-carboxamide compound was obtained as a white solid.

¹H NMR (CDCl₃) δ 0.66 (m, 2H), 0.84 (m, 2H), 2.96 (m, 1H), 7.26 (m, 1H), 7.37 (d, 1H), 7.45-7.48 (m, 2H), 7.58-7.62 (m, 2H), 7.69 (d, 1H), 8.16 (d, 1H), 8.31 (s, 1H), 8.69 (m, 1H), 8.79 (dd, 1H), 8.99 (s, 1H), 9.73 (br, NH).

### EXAMPLE 17D

### N-Isopropyl-1-[3-(3-amino-3-ethylpent-1-ynyl)phenyl]-1,4-dihydro[1,8]naphthyridin-4-one-3-carboxamide

Following the procedure of EXAMPLE 15D, but substituting 1,1-diethylpropargylamine for 2-phenyl-3-butyn-2-ol, the title compound was obtained as a solid.

¹H NMR (CDCl₃) δ 1.05 (t, 6H), 1.28 (d, 6H), 1.57 (m, 2H), 1.69 (m, 2H), 4.27 (m, 1H), 7.33 (d, 1H), 7.44-7.49 (m, 3H), 7.53 (d, 1H), 8.69 (m, 1H), 8.79 (dd, 1H), 8.97 (s, 1H), 9.63 (br, NH). (NH₂ not observed).

### EXAMPLE 8D

### N-Cyclopropyl-1-[3-(3-amino-3-ethylpent-1-ynyl)phenyl]-1,4-dihydro[1,8]naphthyridin-4-one-3-carboxamide

Following the procedure of EXAMPLE 17D, but substituting N-cyclopropyl-1-(3-bromophenyl)-1,4-dihydro[1,8]naphthyridin-4-one-3-carboxamide from Step 1 of EXAMPLE 6D for N-isopropyl-1-(3-bromophenyl)-1,4-dihydro[1,8]naphthyridin-4-one-3-carboxamide, the title compound was obtained as a solid.

¹H NMR (CDCl₃) δ 0.66 (m, 2H), 0.84 (m, 2H), 1.05 (t, 6H), 1.57 (m, 2H), 1.70 (m, 2H), 2.96 (m, 1H), 7.33 (d, 1H), 7.44-7.49 (m, 3H), 7.54 (d, 1H), 8.69 (m, 1H), 8.77 (dd, 1H), 8.97 (s, 1H), 9.75 (br, NH). (NH₂ not observed).

### EXAMPLE 19D

### N-Isopropyl-1-[3-(3-quinolinylethynyl)phenyl]-1,4-dihydro[1,8]naphthyridin-4-one-3-carboxamide

Following the procedure of EXAMPLE 15D, but substituting N-isopropyl-1-(3-ethynylphenyl)-1,4-dihydro[1,8]naphthyridin-4-one-3-carboxamide from Step 2 of EXAMPLE 5D for 2-phenyl-3-butyn-2-ol, and 3-bromoquinoline for N-isopropyl-1-(3-bromophenyl)-1,4-dihydro[1,8]naphthyridin-4-one-3-carboxamide, the title compound was obtained as a solid.

¹H NMR (CDCl₃) δ 1.32 (d, 6H), 4.32 (m, 1H), 7.48-7.51 (m, 2H), 7.58-7.65 (m, 2H), 7.71 (s, 1H), 7.73-7.80 (m, 2H), 7.83 (d, 1H), 8.12 (d, 1H), 8.35 (s, 1H), 8.75 (m, 1H), 8.85 (dd, 1H), 9.02 (s, 1H), 9.06 (s, 1H), 9.65 (br, NH).

### EXAMPLE 20D

### N-Isopropyl-1-[3-(1-oxido-3-quinolinylethynyl)phenyl-1,4-dihydro[1,8]naphthyridin-4-one-3-carboxamide

Following the procedure of EXAMPLE 19D, but substituting 3-bromoquinoline N-oxide for 3-bromoquinoline, the title compound was obtained as a solid.

¹H NMR (CDCl₃) δ 1.33 (d, 6H), 4.32 (m, 1H), 7.49-7.53 (m, 2H), 7.63 (t, 1H), 7.68-7.73 (m, 2H), 7.75-7.83 (m, 2H), 7.88-7.92 (m, 2H), 8.63 (s, 1H), 8.73-8.78 (m, 2H), 8.86 (dd, 1H), 9.05 (s, 1H), 9.67 (br, NH).

### EXAMPLE 21D

### N-Isopropyl-1-[3-(cyclopropylethynyl)phenyl]-1,4-dihydro[1,8]naphthyridin-4-one-3-carboxamide

Following the procedure of EXAMPLE 15D, but substituting cyclopropylacetylene (Tetrahedron letters 2000, 41, 4007) for 2-phenyl-3-butyn-2-ol, the title compound was obtained as a gray solid.

¹H NMR (CDCl₃) δ 0.83 (m, 2H), 0.90 (m, 2H), 1.31 (d, 6H), 1.48 (m, 1H), 4.31 (m, 1H), 7.33 (m, 1H), 7.45-7.51 (m, 3H), 7.55 (d, 1H), 8.72 (m, 1H), 8.83 (dd, 1H), 9.01 (s, 1H), 9.68 (br, NH).

### EXAMPLE 22D

### N-Isopropyl-1-[3-(6-amino-3-pyridinylethynyl)phenyl]-1,4-dihydro[1,8]naphthyidin-4-one-3-carboxamide

Following the procedure of EXAMPLE 19D, but substituting but substituting 5-bromo-2-aminopyridine for 3-bromoquinoline, the title compound was obtained as a solid.

¹H NMR (CDCl₃) δ 1.33 (d, 6H), 4.31 (m, 1H), 4.71 (br, NH₂), 6.49 (d, 1H), 7.40 (m, 1H), 7.48 (m, 1H), 7.54-7.60 (m, 3H), 7.68 (d, 1H), 8.28 (s, 1H), 8.72 (m, 1H), 8.83 (dd, 1H), 9.04 (s, 1H), 9.67 (br, NH).

### EXAMPLE 23D

### N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide

### Step 1: 3-Bromo-5-(1-hydroxy-1-methylethyl)pyridine

To a solution of ethyl 5-bromonicotinate (1.02 g, 4.4 mmol) in diethyl ether (15 mL) at −30° C. was added a 3M solution of methyl magnesium bromide in ether (4mL, 12 mmol). The resulting slurry was refluxed for 2 hours then cooled and quenched with an excess of 0.5M aqueous monobasic sodium phosphate and partitioned between ether and water. The product from the organic phase was chromatographed on silica gel eluting with a 2:1:2 mixture of ether, pentane and ammonia-saturated methylene chloride to afford the 3-bromo-5-(1-hydroxy-1-methylethyl)pyridine compound as a yellow oil.

### Step 2: 3-Bromo-5-(1-hydroxy-1-methylethyl)pyridine-N-oxide

To a solution of 3-bromo-5-(1-hydroxy-1-methylethyl)pyridine from Step 1 (3.1 mmol) in chloroform (10 mL) was added m-chloroperoxybenzoic acid 70% (1.5 eq) and the resulting mixture was stirred at room temperature for 18 hours. An excess of calcium hydroxide was added and after stirring for 5 minutes, the mixture was filtered through celite and the filtrate was evaporated. The crude material was chromatographed on silica gel eluting with 10% ethanol in methylene chloride (saturated with ammonia) to afford the 3-bromo-5-(1-hydroxy-1-methylethyl)pyridine-N-oxide compound as a solid.

### Step 3: N-Isopropyl-{3-[5-(1-hydroxy-1-methylethyl)-1-oxido-3-pyridinylethynyl]phenyl}-1,4-dihydro[1,8]naphthyridin-4-one-3-carboxamide

Following the procedure of EXAMPLE 15D, but substituting N-isopropyl-1-(3-ethynylphenyl)-1,4-dihydro[1,8]naphthyridin-4-one-3-carboxamide from EXAMPLE 5D for 2-phenyl-3-butyn-2-ol, and 3-bromo-5-(1-hydroxy-1-methylethyl)pyridine-N-oxide from Step 2 for N-isopropyl-1-(3-bromophenyl)-1,4-dihydro[1,8]naphthyridin-4-one-3-carboxamide, the N-isopropyl-1-{3-[5-(1-hydroxy-1-methylethyl)-1-oxido-3-pyridinylethynyl]phenyl}-1,4-dihydro[1,8]naphthyridin-4-one-3-carboxamide compound was obtained as a solid.

¹H NMR (CDCl₃) δ 1.32 (d, 6H), 1.64 (s, 6H), 2.22 (br, 1H, OH), 4.30 (m, 1H), 7.45-7.52 (m, 2H), 7.60 (t, 1H), 7.66 (s, 1H), 7.72 (d, 1H), 7.98 (s, 1H), 8.70 (br, 2H), 8.73 (m, 1H), 8.84 (dd, 1H), 9.03 (s, 1H), 9.68 (br, NH).

### EXAMPLE 24D

### N-Isopropyl-1-{3-[6-(1-hydroxy-1-methylethyl)-3-pyridinylethynyl]phenyl}-1,4-dihydro[1,8]naphthyridin-4-one-3-carboxamide

### Step 1: 5-Bromo-2-(1-hydroxy-1-methylethyl)pyridine

To a suspension of 2,5-dibromopyridine in toluene (12mL/mmol) cooled to −78° C. was added n-butyllithium 2.5M in hexanes (1.05 eq) and the resulting mixture was stirred in the cold for 2.5 hours. Acetone (2 eq) was added and stirring was continued for 1.5 h. After quenching with saturated aqueous ammonium chloride solution, the mixture was warmed to room temperature and partitioned between ethyl acetate and water. The product from the organic phase was chromatographed on silica gel eluting with 20% ethyl acetate in hexane to afford the 5-bromo-2-(1-hydroxy-1-methylethyl)pyridine compound as a syrup.

### Step 2: 5-Bromo-2-(1-methyl-1-{[2-(trimethylsilyl)ethoxy]methoxy}ethyl)pyridine

To a solution of 5-bromo-2-(1-hydroxy-1-methylethyl)pyridine from Step 1 (14 mmol) in methylene chloride (50 mL) at 0° C. was added N,N-diisopropylethylamine (37.3 mmol) and 2-(trimethylsilyl)ethoxymethyl chloride (15.3 mmol). The resulting mixture was stirred at room temperature for 18 hours, then refluxed for 24 hours. After cooling to room temperature the mixture was quenched with saturated aqueous ammonium chloride solution and partitioned between methylene chloride and water. The crude product from the organic phase was chromatographed on silica gel eluting with 6% ethyl acetate in hexane to afford the 5-bromo-2-(1-methyl-1-{[2-(trimethylsilyl)ethoxy]methoxy}ethyl)pyridine compound.

### Step 3: 2-(1-Methyl-1-{[2-(trimethylsilyl)ethoxy]methoxy}ethyl)-5-[(trimethylsilyl)ethynyl]pyridine

Following the procedure of Step 5 of EXAMPLE 1D, but substituting the product from present Step 2 for N-isopropyl-1-(3-bromophenyl)-1,4-dihydro[1,8]naphthyridin-4-one-3-carboxamide and trimethylsilylacetylene for phenylacetylene, the 2-(1-methyl-1-{[2-(trimethylsilyl)ethoxy]methoxy)ethyl)-5-[(trimethylsilyl)ethynyl]pyridine compound was obtained.

### Step 4: 5-Ethynyl-2-(1-methyl-1-{[2-(trimethylsilyl)ethoxy]methoxy}ethyl)pyridine

Following the procedure of Step 2 of EXAMPLE 5D, but substituting the product from present Step 3 for N-isopropyl-1-[3-(trimethylsilylethynyl)phenyl]-1,4-dihydro[1,8]naphthyridin-4-one-3-carboxamide, the 5-ethynyl-2-(1-methyl-1-{[2-(trimethylsilyl)ethoxy]methoxy}ethyl)pyridine compound was obtained.

### Step 5: N-Isopropyl-1-(3-{[6-(1-methyl-1-{{2-trimethylsilyl)ethoxy]methoxy}ethyl)pyridin-3-yl]ethynyl}phenyl)-1,4-dihydro[1,8]naphthyridin-4-one-3-carboxamide

Following the procedure of Step 5 of EXAMPLE 1D, but substituting the product from present Step 4 for phenylacetylene, the N-isopropyl-1-(3-{[6-(1-methyl-1-{{2-trimethylsilyl)ethoxy]methoxy}ethyl)pyridin-3-yl]ethynyl}phenyl)-1,4-dihydro[1,8]naphthyridin-4-one-3-carboxamide compound was obtained.

### Step 6: N-Isopropyl-1-{3-[6-(1-hydroxy-1-methylethyl)-3-pyridinylethynyl]phenyl}-1,4-dihydro[1,8]naphthyridin-4-one-3-carboxamide

To a solution of N-isopropyl-1-(3-{[6-(1-methyl-1-{{2-trimethylsilyl)ethoxy]methoxy}ethyl)pyridin-3-yl]ethynyl}phenyl)-1,4-dihydro[1,8]naphthyridin-4-one-3-carboxamide product from present Step 5 in methylene chloride (3.2 mL/mmol) at 0° C. was added trifluoroacetic acid (3.2 mL/mmol). The resulting mixture was stirred at 0° C. for 2 hours then at room temperature for 2 hours. The mixture was neutralized slowly with saturated aqueous sodium bicarbonate and partitioned between methylene chloride and water. The crude material from the organic phase was chromatographed on silica gel eluting with 40% ether in methylene chloride and the purified product was stirred in ether at room temperature for 2 hours and filtered to afford the N-isopropyl-1-{3-[6-(1-hydroxy-1-methylethyl)-3-pyridinylethynyl]phenyl}-1,4-dihydro[1,8]naphthyridin-4-one-3-carboxamide compound as solid.

¹H NMR (Acetone-d₆) δ 1.24 (d, 6H), 1.50 (s, 6H), 4.18 (m, 1H), 4.57 (s, 1H, OH), 7.61 (m, 1H), 7.69-7.74 (m, 3H), 7.78 (m, 1H), 7.88 (s, 1H), 7.93 (dd, 1H), 8.68 (s, 1H), 8.74 (m, 1H), 8.78 (dd, 1H), 8.88 (s, 1H), 9.63 (br, NH).

### EXAMPLE 25D

### N-Isopropyl-1-{3-[6-(1-hydroxy-1-methylethyl)-1-oxido-3-pyridinylethynyl]phenyl}-1,4-dihydro[1,8]naphthyridin-4-one-3-carboxamide

Following the procedure of Step 2 of EXAMPLE 23D, but substituting N-isopropyl-1-{3-[6-(1-hydroxy-1-methylethyl)-3-pyridinylethynyl]phenyl}-1,4-dihydro[1,8]naphthyridin-4-one-3-carboxamide from example 24D for 3-bromo-5-(1-hydroxy-1-methylethyl)pyridine, the title compound was obtained as a solid.

¹H NMR (Acetone-4) δ 1.25 (d, 6H), 1.60 (s, 61), 4.18 (m, 1H), 7.24 (s, 1H, OH), 7.60-7.63 (m, 3H), 7.72-7.78 (m, 2H), 7.82 (d, 1H), 7.91 (s, 1H), 8.46 (s, 1H), 8.74 (m, 1H), 8.78 (dd, 1H), 8.87 (s, 1H), 9.62 (br, NH).

### EXAMPLE 26D

### N-Isopropyl-1-{3-[4(1-hydroxy-1-methylethyl)-2-pyridinylethynyl]phenyl-1,4-dihydro[1,8]naphthyridin-4-one-3-carboxamide

### Step 1: Methyl 2-bromoisonicotinate

To a solution of 2-bromoisonicotinic acid (Chem. Pharm. Bull. 1990, 38, 2446) (2.0 g) in tetrahydrofuran (100 mL) was added excess ethereal diazomethane and the resulting mixture was stirred at room temperature for 1 hour. The mixture was evaporated and the product chromatographed on silica gel eluting with a 1:3 mixture of ethyl acetate and hexane to afford the methyl 2-bromoisonicotinate ester as a colorless liquid.

### Step 2: 2-Bromo-4-(1-hydroxy-1-methylethyl)pyridine

Following the procedure of Step 1 of EXAMPLE 23D, but substituting methyl 2-bromoisonicotinate from present Step 1 for ethyl 5-bromonicotinate, the 2-bromo-4-(1-hydroxy-1-methylethyl)pyridine compound was obtained as a white solid.

### Step 3: N-Isopropyl-1-{3-[4-(1-hydroxy-1-methylethyl)-2-pyridinylethynyl]phenyl}-1,4-dihydro[1,8]naphthyridin-4-one-3-carboxamide

Following the procedure of EXAMPLE 19D, but substituting the 2-bromo-4-(1-hydroxy-1-methylethyl)pyridine from present Step 2 for 3-bromoquinoline, the N-isopropyl-1-{3-[4-(1-hydroxy-1-methylethyl)-2-pyridinylethynyl]phenyl}-1,4-dihydro[1,8]naphthyridin-4-one-3-carboxamide compound was obtained as a yellow foam.

¹H NMR (Acetone-d₆) δ 1.27 (d, 6H), 1.55 (s, 6H), 4.20 (m, 1H), 4.42 (s, 1H, OH), 7.52 (m, 1H), 7.63 (m, 1H), 7.72-7.79 (m, 3H), 7.84 (d, 1H), 7.95 (s, 1H), 8.55 (d, 1H), 8.77 (m, 1H), 8.80 (dd, 1H), 8.92 (s, 1H), 9.65 (br, NH).

### EXAMPLE 27D

### N-Isopropyl-1-{3-[5-(1-hydroxy-1-methylethyl)-2-pyridinylethynyl]phenyl}-1,4-dihydro[1,8]naphthyridin-4-one-3-carboxamide

### Step 1: 2-Bromo-5-(1-hydroxy-1-methylethyl)pyridine

A solution of 2,5-dibromopyridine in diethyl ether (5 mL/mmol) was cooled to −78° C., and n-butyllithium 2.5M in hexanes (1.05 eq) was added slowly. After 2 hrs in the cold, acetone (1.3 eq) was added and stirring was continued for 1 hour. The resulting mixture was quenched with saturated aqueous ammonium chloride solution, warmed to room temperature, and partitioned between ether and water. The crude product from the organic phase was triturated with 1:1 ether-hexane and filtered to afford the 2-bromo-5-(1-hydroxy-1-methylethyl)pyridine compound as a solid.

### Step 2: 5-(1-Hydroxy-1-methylethyl)-2-[(trimethylsilyl)ethynyl]pyridine

Following the procedure of EXAMPLE 15D, but substituting the product 2-bromo-5-(1-hydroxy-1-methylethyl)pyridine from present Step 1 for N-isopropyl-1-(3-bromophenyl)-1,4-dihydro[1,8]naphthyridin-4-one-3-carboxamide and trimethylsilylacetylene for 2-phenyl-3-butyn-2-ol, the 5-(1-hydroxy-1-methylethyl)-2-[(trimethylsilyl)ethynyl]pyridine compound was obtained.

### Step 3: 2-Ethynyl-5-(1-hydroxy-1-methylethyl)pyridine

Following the procedure of Step 2 of EXAMPLE 5D, but substituting the product 5-(1-hydroxy-1-methylethyl)-2-[(trimethylsilyl)ethynyl]pyridine from present Step 2 for N-isopropyl-1-[3-(trimethylsilylethynyl)phenyl]-1,4-dihydro[1,8]naphthyridin-4-one-3-carboxamide, the 2-ethynyl-5-(1-hydroxy-1-methylethyl)pyridine compound was obtained.

### Step 4: N-Isopropyl-1-{3-[5-(1-hydroxy-1-methylethyl)-2-pyridinylethynyl]phenyl}-1,4-dihydro[1,8]naphthyridin-4-one-3-carboxamide

Following the procedure of EXAMPLE 15D but substituting the product 2-ethynyl-5-(1-hydroxy-1-methylethyl)pyridine from present Step 3 for 2-phenyl-3-butyn-2-ol, the N-Isopropyl-1-{3-[5-(1-hydroxy-1-methylethyl)-2-pyridinylethynyl]phenyl}-1,4-dihydro[1,8]naphthyridin-4-one-3-carboxamide compound was obtained.

¹H NMR (CDCl₃) δ 1.32 (d, 6H), 1.66 (s, 6H), 2.08 (s, 1H, OH), 4.31 (m, 1H), 7.46-7.55 (m, 3H), 7.61 (t, 1H), 7.71 (s, 1H), 7.78 (d, 1H), 7.86 (dd, 1H), 8.73 (m, 1H), 8.77 (m, 1H), 8.83 (dd, 1H), 9.04 (s, 1H), 9.67 (br, NH).

### EXAMPLE 28D

### N-Isopropyl-1-{3-[6-(1-hydroxy-1-methylethyl)-2-pyridinylethynyl]phenyl}-1,4-dihydro[1,8]naphthyridin-4-one-3-carboxamide

### Step 1: 2-Bromo-6-(1-hydroxy-1-methylethyl)pyridine

Following the procedure of Step 1 of EXAMPLE 27D, but substituting 2,6-dibromopyridine for 2,5-dibromopyridine, the 2-Bromo-6-(1-hydroxy-1-methylethyl)pyridine compound was obtained as a solid.

### Step 2: N-Isopropyl-1-{3-[6-(1-hydroxy-1-methylethyl)-2-pyridinylethynyl]phenyl}-1,4-dihydro[1,8]naphthyridin-4-one-3-carboxamide

Following the procedure of EXAMPLE 19D, but substituting the product 2-Bromo-6-(1-hydroxy-1-methylethyl)pyridine from present Step 1 for 3-bromoquinoline, the N-Isopropyl-1-{3-[6-(1-hydroxy-1-methylethyl)-2-pyridinylethynyl]phenyl}-1,4-dihydro[1,8]naphthyridin-4-one-3-carboxamide compound was obtained.

¹H NMR (CDCl₃) δ 1.31 (d, 6H), 1.58 (s, 6H), 4.32 (m, 1H), 4.83 (s, 1H, OH), 7.38 (d, 1H), 7.43-7.52 (m, 3H), 7.60 (t, 1H), 7.70-7.75 (m, 2H), 7.79 (d, 1H), 8.74 (m, 1H), 8.84 (dd, 1H), 9.03 (s, 1H), 9.66 (br, NH).

### EXAMPLE 29D

### N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide

### Step 1: 5-Bromo-2-(1-hydroxy-1-methylethyl)pyridine N-oxide

Following the procedure of Step 2 of EXAMPLE 23D, but substituting 5-bromo-2-(1-hydroxy-1-methylethyl)pyridine from Step 1 of EXAMPLE 24D for 3-bromo-5-(1-hydroxy-1-methylethyl)pyridine, the 5-Bromo-2-(1-hydroxy-1-methylethyl)pyridine N-oxide compound was obtained.

### Step 2: N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide

Following the procedure of EXAMPLE 15D, but substituting N-cyclopropyl-1-(3-ethynylphenyl)-1,4-dihydro[1,8]naphthyridin-4-one-3-carboxamide from EXAMPLE 6D for 2-phenyl-3-butyn-2-ol and 5-bromo-2-(1-hydroxy-1-methylethyl)pyridine-N-oxide from present Step 1 for N-isopropyl-1-(3-bromophenyl)-1,4-dihydro[1,8]naphthyridin-4-one-3-carboxamide, the N-cyclopropyl-1-{3-[6-(1-hydroxy-1-methylethyl)-1-oxido-3-pyridinylethynyl]phenyl}-1,4-dihydro[1,8]naphthyridin-4-one-3-carboxamide compound was obtained as a solid.

¹H NMR (CDCl₃) δ 0.66 (m, 2H), 0.84 (m, 2H), 1.66 (s, 6H), 2.96 (m, 1H), 7.34 (d, 1H), 7.43-7.50 (m, 4H), 7.58-7.62 (m, 2H), 7.69 (d, 1H), 8.33 (s, 1H, OH), 8.69 (m, 1H), 8.79 (dd, 1H), 8.99 (s, 1H), 9.73 (br, NH).

### EXAMPLE 30D

### N-Isopropyl-1-{3-[(4-pyridin-3-ylphenyl)ethynyl]phenyl}-1,4-dihydro[1,8]naphthyridin-4-one-3-carboxamide

### Step 1: 3-(4-Bromophenyl)pyridine

A mixture of pyridine-3-boronic acid 1,3-propanediol cyclic ester, 4-bromoiodobenzene (1.1 eq), [1,1′-bis (diphenylphosphino)ferrocene]dichloropalladium(II) (0.05 eq) and 2M aqueous sodium carbonate (5 eq) in N,N-dimethylformamide (2 mL/mmol) was stirred at 85° C. for 4 hours. After quenching with saturated aqueous ammonium chloride solution the mixture was partitioned between ethyl acetate and water and the crude product from the organic phase was chromatographed on silica gel eluting with a 1:9 mixture of ethyl acetate and hexane to afford the 3-(4-bromophenyl)pyridine compound as a solid.

### Step 2: N-Isopropyl-1-{3-[(4-pyridin-3-ylphenyl)ethynyl]phenyl}-1,4-dihydro[1,8]naphthyridin-4-one-3-carboxamide

Following the procedure of EXAMPLE 19D, but substituting the product from present Step 1 for 3-bromoquinoline, the N-isopropyl-1-{3-[(4-pyridin-3-ylphenyl)ethynyl]phenyl}-1,4-dihydro[1,8]naphthyridin-4-one-3-carboxamide compound was obtained.

¹H NMR (CDCl₃) δ 1.28 (d, 6H), 4.28 (m, 1H), 7.38 (m, 1H), 7.42 (d, 1H), 7.48 (m, 1H), 7.53-7.64 (m, 6H), 7.70 (d, 1H), 7.88 (d, 1H), 8.60 (m, 1H), 8.71 (m, 1H), 8.82 (dd, 1H), 8.86 (s, 1H), 9.02 (s, 1H), 9.63 (br, NH).

### EXAMPLE 31D

### N-Isopropyl-1-(3-{[5-(1-hydroxy-1-methylethyl)thien-2-yl]ethynyl}phenyl)-1,4-dihydro[1,8]naphthyridin-4-one-3-carboxamide

### Step 1: 2-Bromo-5-(1-hydroxy 1—methylethyl)thiophene

To a solution of 2-acetyl-5-bromothiophene in THF (2.5 mL/mmol) at −30° C. was added 1.4M methylmagnesium bromide in 3:1 toluene-TBF (1.5 eq) and the resulting mixture was warned to −10° C. and stirred for 1.5 hours. After quenching with saturated aqueous ammonium chloride solution, the mixture was partitioned between ether and water. The organic fraction was dried and evaporated, and the crude material was chromatographed on silica gel eluting with a 1:4 mixture of ether and hexane to afford the 2-bromo-5-(1-hydroxy-1-methylethyl)thiophene compound.

Step 2: 2-(1-Hydroxy-1-methylethyl)-5-trimethylsilylethynyl thiophene

Following the procedure of EXAMPLE 15D, but substituting the product from present Step 1 for N-isopropyl-1-(3-bromophenyl)-1,4-dihydro[1,8]naphthyridin-4-one-3-carboxamide and trimethylsilylacetylene for 2-phenyl-3-butyn-2-ol, the 2-(1-hydroxy-1-methylethyl)-5-trimethylsilylethynyl thiophene compound was obtained.

### Step 3: 2-Ethynyl-5-(1-hydroxy-1-methylethyl)thiophene

Following the procedure of Step 2 of EXAMPLE 5D, but substituting the product from present Step 2 for N-isopropyl-1-[3-(trimethylsilylethynyl)phenyl]-1,4-dihydro[1,8]naphthyridin-4-one-3-carboxamide, the 2-ethynyl-5-(1-hydroxy-1-methylethyl)thiophene compound was obtained.

### Step 4: N-Isopropyl-1-(3-{[5-(1-hydroxy-1-methylethyl)thien-2-yl]ethynyl}phenyl-1,4-dihydro[1,8]naphthyridin-4-one-3-carboxamide

Following the procedure of EXAMPLE 15D, but substituting the 2-ethynyl-5-(1-hydroxy-1-methylethyl)thiophene product from present Step 3 for 2-phenyl-3-butyn-2-ol, the N-isopropyl-1-(3-{[5-(1-hydroxy-1-methylethyl)thien-2-yl]ethynyl}phenyl)-1,4-dihydro[1,8]naphthyridin-4-one-3-carboxamide compound was obtained as a solid.

¹H NMR (CDCl₃) δ 1.31 (d, 6H), 1.70 (s, 6H), 2.42 (s, 1H, OH), 4.31 (m, 1H), 6.87 (d, 1H), 7.16 (d, 1H), 7.42 (d, 1H), 7.48 (m, 1H), 7.59 (t, 1H), 7.63 (s, 1H), 7.68 (d, 1H), 8.73 (m, 1H), 8.84 (dd, 1H), 9.02 (s, 1H), 9.68 (br, NH).

### EXAMPLE 32D

### N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide

### Step 1: 2-(1-hydroxy-1-methylethyl)thiazole

To a solution of thiazole in ether (1 mL/mmol) at −78° C. was added 2.2M n-butyllithium in hexanes (1.1 eq) and the resulting mixture was stirred for 30 minutes. Acetone (1.2 eq) was added and the mixture was stirred at −78° C. for a further 30 minutes. The mixture was quenched in the cold with saturated aqueous ammonium chloride solution and warmed to room temperature, then partitioned between ether and water. The organic phase was dried and evaporated to yield the crude product as an orange-brown oil which was used as such in the next step.

### Step 2: 2-(1-methyl-1-{[2-(trimethylsilyl)ethoxy]methoxy}ethyl)thiazole

Following the procedure of Step 2 of EXAMPLE 24D, but substituting the product from present Step 1 for 5-bromo-2-(1-hydroxy-1-methylethyl)pyridine, the 2-(1-methyl-1-{[2-(trimethylsilyl)ethoxy]methoxy}ethyl)thiazole compound was obtained as an oil.

### Step 3: 5-Bromo-2-(1-hydroxy-1-methylethyl)thiazole

To a solution of 2-(1-methyl-1-{[2-(trimethylsilyl)ethoxy]methoxy)ethyl)thiazole from Step 2 in chloroform (2 mL/mmol) at room temperature was added bromine (2 molar eq) and the resulting mixture was stirred for 1 hour. Solid sodium bicarbonate (0.55 eq) was added and the mixture was stirred for 5 hours. More sodium bicarbonate was added (0.55 eq) and stirring was continued for 18 hours. After a final addition of sodium bicarbonate (0.55 eq) the mixture was stirred for a further 5 hours, diluted with chloroform and the organic phase was washed with saturated aqueous sodium bicarbonate, then with water, dried and evaporated. The crude material was chromatographed, eluting with a 3:7 mixture of ethyl acetate and hexane to afford the desired product.

### Step 4: N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide

Following the procedure of EXAMPLE 19D, but substituting the product from present Step 3 for 3-bromoquinoline, the N-isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide compound was obtained as a solid.

¹H NMR (CDCl₃) δ 1.29 (d, 6H), 1.68 (s, 6H), 2.90 (s, 1H, OH), 4.28 (m, 1H), 7.42 (d, 1H), 7.46 (m, 1H), 7.54-7.60 (m, 2H), 7.66 (d, 1H), 7.82 (s, 1H), 8.70 (m, 1H), 8.80 (dd, 1H), 8.99 (s, 1H), 9.63 (br, NH).

### EXAMPLE 33D

### 1-[3-(1-Oxido-3-pyridinylethynyl)phenyl]-1,4-dihydro[1,8]naphthyridin-4-one-3-carboxamide

### Step 1:1-(3-Bromophenyl)-1,4-dihydro[1,8]naphthyridin-4-one-3-carboxamide

Following the procedure of Step 4 of EXAMPLE 1D, but substituting 28% aqueous ammonium hydroxide for isopropylamine, the 1-(3-bromophenyl)-1,4-dihydro[1,8]naphthyridin-4-one-3-carboxamide compound was obtained as a solid.

### Step 2: 1-[3-(Trimethylsilylethvnyl)phenyl]-1,4-dihydro[1,8]naphthyridin-4-one-3-carboxamide

Following the procedure of Step 5 of EXAMPLE 1D, but substituting the 1-(3-Bromophenyl)-1,4-dihydro[1,8]naphthyridin-4-one-3-carboxamide from present Step 1 for N-isopropyl-1-(3-bromophenyl)-1,4-dihydro[1,8]naphthyridin-4-one-3-carboxamide and trimethylsilylacetylene for phenylacetylene, the 1-[3-(trimethylsilylethynyl)phenyl]-1,4-dihydro[1,8]naphthyridin-4-one-3-carboxamide product was obtained as a solid.

### Step 3: 1-(3-Ethynylphenyl)-1,4-dihydro[1,8]naphthyridin-4-one-3-carboxamide

To a solution of 1-[3-(trimethylsilylethynyl)phenyl]-1,4-dihydro[1,8]naphthyridin-4-one-3-carboxamide from Step 2 in THF (30 mL/mmol) at 0° C. was added 1M tetrabutylammonium fluoride in THF (1.5 eq) and the resulting mixture was stirred at 0° C. for 30 minutes. The mixture was partitioned between methylene chloride and water and the organic phase was dried and evaporated. The crude 1-(3-ethynylphenyl)-1,4-dihydro[1,8]naphthyridin-4-one-3-carboxamide product was used as such in the next step.

### Step 4: 1-[3-(1-oxido-3-pyridinylethynyl)phenyl]-1,4-dihydro[1,8]naphthyridin-4-one-3-carboxamide

Following the procedure of example 19D, but substituting the 1-(3-ethynylphenyl)-1,4-dihydro[1,8]naphthyridin-4-one-3-carboxamide from Step 3 for N-Isopropyl-1-(3-ethynylphenyl)-1,4-dihydro[1,8]naphthyridin-4-one-3-carboxamide and 3-bromopyridine N-oxide for 3-bromoquinoline, the 1-[3-(1-oxido-3-pyridinylethynyl)phenyl]-1,4-dihydro[1,8]naphthyridin-4-one-3-carboxamide was obtained as a white solid.

¹H NMR (CDCl₃) δ 5.84 (br, 1H, NH), 7.30 (m, 1H), 7.41 (d, 1H), 7.53 (m, 2H), 7.64 (t, 1H), 7.67 (s, 1H), 7.74 (d, 1H), 8.21 (d, 1H), 8.35 (s, 1H), 8.75 (m, 1H), 8.88 (dd, 1H), 9.05 (s, 1H), 9.52 (br, 1H, NH).

### EXAMPLE 34D

### 1-[3-(1-Oxido-3-pyridinylethynyl)phenyl]-1,4-dihydro[1,8]naphthyridin-4-one-3-carboxylic acid

### Step 1: Ethyl 1-(3-Ethynylphenyl)-1,4-dihydro[1,8]naphthyridin-4-one-3-carboxylate

Following the procedures of Steps 1 and 2 of EXAMPLE 5D, but substituting ethyl 1-(3-bromophenyl)-1,4-dihydro[1,8]naphthyridin-4-one-3-carboxylate from Step 2 of EXAMPLE 1 for N-isopropyl-1-(3-bromophenyl)-1,4-dihydro[1,8]naphthyridin-4-one-3-carboxamide as the starting material, the Ethyl 1-(3-Ethynylphenyl)-1,4-dihydro[1,8]naphthyridin-4-one-3-carboxylate compound was obtained as a solid.

### Step 2: Ethyl 1-[3-(1-oxido-3-pyridinylethynyl)phenyl]-1,4-dihydro[1,8]naphthyridin-4-one-3-carboxylate

Following the procedure of EXAMPLE 15D, but substituting the ethyl 1-(3-ethynylphenyl)-1,4-dihydro[1,8]naphthyridin-4-one-3-carboxylate from present Step 1 for 2-phenyl-3-butyn-2-ol and 3-bromopyridine N-oxide for N-isopropyl-1-(3-bromophenyl)-1,4-dihydro[1,8]naphthyridin-4-one-3-carboxamide, the ethyl 1-[3-(1-oxido-3-pyridinylethynyl)phenyl]-1,4-dihydro[1,8]naphthyridin-4-one-3-carboxylate was obtained as a solid.

### Step 3: 1-[3-(1-Oxido-3-pyridinylethynyl)phenyl]-1,4-dihydro[1,8]naphthyridin-4-one-3-carboxylic acid

Following the procedure of Step 3 of EXAMPLE 1D, but substituting the ethyl 1-[3-(1-oxido-3-pyridinylethynyl)phenyl]-1,4-dihydro[1,8]naphthyridin-4-one-3-carboxylate ester from present Step 2 for ethyl 1-(3-bromophenyl)-1,4-dihydro[1,8]naphthyridin-4-one-3-carboxylate, the 1-[3-(1-oxido-3-pyridinylethynyl)phenyl]-1,4-dihydro[1,8]naphthyridin-4-one-3-carboxylic acid was obtained as a white solid.

¹H NMR (DMSO-d₆) δ 7.46 (t, 1H), 7.51 (d, 1H), 7.70 (t, 1H), 7.75 (m, 2H), 7.80 (d, 1H), 7.92 (s, 1H), 8.26 (d, 1H), 8.47 (s, 1H), 8.81 (dd, 1H), 8.89 (m, 1H), 8.97 (s, 1H).

Other variations or modifications, which will be obvious to those skilled in the art, are within the scope and teachings of this invention. This invention is not to be limited except as set forth in the following claims.

1. A method of enhancing cognition in a healthy subject comprising administering a safe cognition enhancing amount of compound selected from the group consisting of:
6-isopropyl-8-(3-{(Z/E)-2-[4-(methylsulfonyl)phenyl]-2-phenylethenyl}phenyl)quinoline;
6-isopropyl-8-{3-[(E/Z)-2-[4-(methylsulfonyl)phenyl]-2-(1,3-thiazol-2-yl)ethenyl]phenyl}quinoline;
6-isopropyl-8-(3-{(E)-2-(1-methyl-1H-imidazol-2-yl)-2-[4-(methylsulfonyl)phenyl]ethenyl}phenyl)quinoline;
6-isopropyl-8-(3-{(Z/E)-2-(4-fluorophenyl)-2-[4-(methylsulfonyl)phenyl]ethenyl}phenyl)quinoline;
2-(2-(E/Z)-2-[3-(6-isopropyl-8-quinolinyl)phenyl]-1-[4-(methylsulfonyl)phenyl]ethenyl}-1,3-thiazol-5-yl)-2-propanol;
2-[8-(3-{(E/Z)-2-[5-(1-hydroxy-1-methylethyl)-1,3-thiazol-2-yl]-2-[4-(methylsulfonyl)phenyl]ethenyl}phenyl)-6-quinolinyl]-2-methylpropanenitrile;
2-methyl-2-[8-(3-{(E)-2-(1-methyl-1H-imidazol-2-yl)-2-[4-(methylsulfonyl)phenyl]ethenyl}phenyl)-6-quinolinyl]propanenitrile;
6-[1-(methylsulfonyl)ethyl]-8-{3-[(E)-2-[4-(methylsulfonyl)phenyl]-2-(1,3-thiazol-2-yl)ethenyl]phenyl}quinoline;
6-[1-methyl-1-(methylsulfonyl)ethyl]-8-{3-[(E)-2-[4-(methylsulfonyl)phenyl]-2-(1,3-thiazol-2-yl)ethenyl]phenyl}quinoline;
8-(3-{(Z)-2-(1-methyl-1H-imidazol-2-yl)-2-[4-(methylsulfonyl)phenyl]ethenyl}phenyl)-6-[1-(methylsulfonyl)ethyl]quinoline;
8-(3-{(Z)-2-(1-methyl-1H-imidazol-2-yl)-2-[4-(methylsulfonyl)phenyl]ethenyl}phenyl)-6-[1-methyl-1-(methylsulfonyl)ethyl]quinoline;
6-[1-methyl-1-(methylsulfonyl)ethyl]-8-(3-{(E/Z)-2-(3-methyl-1,2,4-oxadiazol-5-yl)-2-[4-(methylsulfonyl)phenyl]ethenyl}phenyl)quinoline;
(E/Z)-3-{3-[6-(1-cyano-1-methylethyl)-8-quinolinyl]phenyl}-N-isopropyl-2-[4-(methylsulfonyl)phenyl]-2-propenamide;
8-(3-{(E)-2-{3-[(4-methoxyphenoxy)methyl]-1,2,4-oxadiazol-5-yl}-2-[4-(methylsulfonyl)phenyl]ethenyl}phenyl)-6-[1-methyl-1-(methylsulfonyl)ethyl]quinoline;
(5-{(E)-2-(3-{6-[1-methyl-1-(methylsulfonyl)ethyl]-8-quinolinyl}phenyl)-1-[4-(methylsulfonyl)phenyl]ethenyl}-1,2,4-oxadiazol-3-yl)methanol;
(E)-N-isopropyl-3-(3-{6-[1-methyl-1-(methyl sulfonyl)ethyl]-8-quinolinyl}phenyl)-2-[4-(methylsulfonyl)phenyl]-2-propenamide;
(E)-3-{3-[6-(1-cyano-1-methylethyl)-8-quinolinyl]phenyl}-2-[4-(methylsulfonyl)phenyl]-2-propenoic acid;
2-methyl-2-[8-(3-{(E)-2-(3-methyl-1,2,4-oxadiazol-5-yl)-2-[4-(methylsulfonyl)phenyl]ethenyl}phenyl)-6-quinolinyl]propanenitrile;
(E)-3-{3-[6-(1-cyano-1-methylethyl)-8-quinolinyl]phenyl}-2-[4-(methylsulfonyl)phenyl]-2-propenamide;
E)-N-(tert-butyl)-3-{3-[6-(1-cyano-1-methylethyl)-8-quinolinyl]phenyl}-2-[4-(methylsulfonyl)phenyl]-2-propenamide;
(E)-3-[3-(6-isopropyl-8-quinolinyl)phenyl]-2-[4-(methylsulfonyl)phenyl]-2-propenoic acid;
6-isopropyl-8-(3-{(E)-2-(3-methyl-1,2,4-oxadiazol-5-yl)-2-[4-(methylsulfonyl)phenyl]ethenyl}phenyl)quinoline;
(E)-3-(3-{6-[1-methyl-1-(methylsulfonyl)ethyl]-8-quinolinyl}phenyl)-2-[4-(methylsulfonyl)phenyl]-1-(1-pyrrolidinyl)-2-propen-1-one;
(E)-N-cyclopropyl-3-(3-{6-[1-methyl-1-(methylsulfonyl)ethyl]-8-quinolinyl phenyl}-2-[4-(methylsulfonyl)phenyl]-2-propenamide;
(E)-N-(tert-butyl)-3-(3-{6-[1-methyl-1-(methylsulfonyl)ethyl]-8-quinolinyl}phenyl)-2-[4-(methylsulfonyl)phenyl]-2-propenamide;
8-{3-[2,2-bis(4-chlorophenyl)vinyl]phenyl}-6-isopropylquinoline;
6-isopropyl-8-(3-{(E/Z)-2-(6-methyl-3-pyridinyl)-2-[4-(methylsulfonyl)phenyl]ethenyl}phenyl)quinoline;
6-isopropyl-8-(3-{(E/Z)-2-(5-methyl-2-pyridinyl)-2-[4-(methylsulfonyl)phenyl]ethenyl}phenyl)quinoline;
8-(3-{2,2-bis[4-(methylsulfonyl)phenyl]vinyl}phenyl)-6-isopropylquinoline;
2-methyl-2-[8-(3-{(E/Z)-2-(5-methyl-2-pyridinyl)-2-[4-(methylsulfonyl)phenyl]ethenyl}phenyl)-6-quinolinyl]propanenitrile;
2-[8-(3-{2,2-bis[4-(methylsulfonyl)phenyl]vinyl}phenyl)-6-quinolinyl]-2-methylpropanenitrile;
2-methyl-2-(8-{3-[(E)-2-[4-(methylsulfonyl)phenyl]-2-(2-pyridinyl)ethenyl]phenyl}-6-quinolinyl)propanenitrile;
6-[1-methyl-1-(methylsulfonyl)ethyl]-8-(3-{(E/Z)-2-(5-methyl-2-pyridinyl)-2-[4-(methylsulfonyl)phenyl]ethenyl}phenyl)quinoline;
2-(6-{(E)-2-(3-{6-[1-methyl-1-(methyl sulfonyl)ethyl]-8-quinolinyl}phenyl)-1-[4-(methylsulfonyl)phenyl]ethenyl}-3-pyridinyl)-2-propanol;
2-[8-(3-{(1E/Z)-3-(dimethylamino)-2-[4-(methylsulfonyl)phenyl]prop-1-enyl}phenyl)quinolin-6-yl]-2-methylpropanenitrile;
(±)-4-{2-[3,4-Bis(difluoromethoxy)phenyl]-2-{5-[2-(1-hydroxy-1-methyl)ethyl]thiazolyl}ethyl}pyridine N-oxide;
chiral 4-{2-[3,4-Bis(difluoromethoxy)phenyl]-2-{5-[2-(1-hydroxy-1-methyl)ethyl]thiazolyl}ethyl}pyridine N-oxide;
(±/±)-4-{2-[3,4-Bis(difluoromethoxy)phenyl]-2-[5-(2-(1-hydroxy-2,2,2-trifluoro)ethyl)thiazolyl]ethyl}pyridine;
(±/±)-4-{2-[3,4-Bis(difluoromethoxy)phenyl]-2-[5-(2-(1-hydroxy-2,2,2-trifluoro)ethyl)thiazolyl]ethyl}pyridine n-oxide;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine;
(±)-4-{2-[3,4-Bis(difluoromethoxy)phenyl]-2-{5-[2-(1-hydroxy-1-trifluoromethyl-2,2,2-trifluoro)ethyl]thiazolyl}ethyl}pyridine n-oxide;
(±/±)-4-{2-[3,4-Bis(difluoromethoxy)phenyl]-2-{5-[2-(1-hydroxy-1-trifluoromethyl)ethyl]thiazolyl}ethyl}pyridine N-oxide;
(±/±)-4-{2-[3,4-Bis(difluoromethoxy)phenyl]-2-[5-(2-phenylmethanol)thiazolyl]ethyl}pyridine n-oxide;
(±/±)-4-{2-[3,4-Bis(difluoromethoxy)phenyl]-2-[5-(2-(1-hydroxy-1-phenyl)ethyl)thiazolyl]ethyl}pyridine n-oxide;
(±/±)-4-{2-[3,4-Bis(difluoromethoxy)phenyl]-2-[5-(2-(1-hydroxy-1-phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-phenyl)propyl)thiazolyl]ethyl}pyridine n-oxide;
(±/±)-4-{2-[3,4-Bis(difluoromethoxy)phenyl]-2-[5-(2-cyclohexylmethanol)thiazolyl]ethyl}pyridine;
(±/±)-4-{2-[3,4-Bis(difluoromethoxy)phenyl]-2-[5-(2-(1-hydroxy-1-cyclohexyl-2,2,2-trifluoromethyl)ethyl)thiazolyl]ethyl}pyridine n-oxide;
(±/±)-4-{2-[3,4-Bis(difluoromethoxy)phenyl]-2-[5-(2-(1-hydroxy-1-(4-ethyl)phenyl)ethyl)thiazolyl]ethyl}pyridine n-oxide;
(±/±)-4-{2-[3,4-Bis(difluoromethoxy)phenyl]-2-[5-(2-(1-hydroxy-1-(4-ethyl)phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(4-fluoro)phenyl)ethyl)thiazolyl]ethyl}pyridine n-oxide;
(±/±)-4-{2-[3,4-Bis(difluoromethoxy)phenyl]-2-[5-(2-(1-hydroxy-1-(4-Fluoro)phenyl-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(5-bromopyridin-2-yl)-2,2,2-trifluoro)ethyl)thiazolyl]ethyl}pyridine n-oxide;
(±/±)-4-{2-[3,4-Bis(difluoromethoxy)phenyl]-2-[5-(2-(1-hydroxy-1-(6-bromopyridin-3-yl)-2,2,2-trifluoro)ethyl)thiazolyl]ethyl}pyridine n-oxide;
(±)-4-{2-[3,4-Bis(difluoromethoxy)phenyl]-2-{5-[2-(1-hydroxy)cyclobutyl]thiazolyl}ethyl}pyridine N-oxide;
(±)-4-{2-[3,4-Bis(difluoromethoxy)phenyl]-2-{5-[2-(1-hydroxy)cyclohexyl]thiazolyl}ethyl}pyridine N-oxide;
(±)-4-{2-[(3-cyclobutyloxy-4-difluoromethoxy)phenyl]-2-{5-[2-(1-hydroxy-1-methyl)ethyl]thiazolyl}ethyl}pyridine N-oxide;
chiral 4-{2-[(3-cyclobutyloxy-4-difluoromethoxy)phenyl]-2-{5-[2-(1-hydroxy-1-methyl)ethyl]thiazolyl}ethyl}pyridine N-oxide;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine;
(±)-4-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine n-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine;
chiral 3-{2-[(3-cyclobutyloxy-4-difluoromethoxy)phenyl]-2-{5-[2-(1-hydroxy-1-methyl)ethyl]thiazolyl}ethyl}pyridine n-oxide;
chiral 3-{2-[(3-cyclobutyloxy-4-difluoromethoxy)phenyl]-2-{5-[2-(1-hydroxy-1-methyl)ethyl]thiazolyl}ethyl}pyridine n-oxide;
chiral 3-{2-[(3-cyclobutyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
(±)-4-{2-[(3-cyclopropyloxy-4-difluoromethoxy)phenyl]-2-{5-[2-(1-hydroxy-1-methyl)ethyl]thiazolyl}ethyl}pyridine N-oxide;
(±)-3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
chiral 3-{2-[(3-cyclopropyloxy-4-difluoromethoxy)phenyl]-2-{5-[2-(1-hydroxy-1-trifluoromethyl-2,2,2-trifluoro)ethyl]thiazolyl}ethyl}pyridine N-oxide;
N-Isopropyl-1-[3-(3-acetylphenyl)phenyl]-1,4-dihydro[1,8]naphthyridin-4-one-3-carboxamide;
N-(2,6-Dichloropyridin-4-yl)-1-[3-(3-acetylphenyl)phenyl]-1,4-dihydro[1,8]naphthyridin-4-one-3-carboxamide;
N-Isopropyl-1-[3-(4-n-propylphenyl)phenyl]-1,4-dihydro[1,8]naphthyridin-4-one-3-carboxamide;
N-Isopropyl-1-[3-(4-acetylphenyl)phenyl]-1,4-dihydro[1,8]naphthyridin-4-one-3-carboxamide
N-Isopropyl-1-[3-(2-methylphenyl)phenyl]-1,4-dihydro[1,8]naphthyridin-4-one-3-carboxamide;
N-Isopropyl-N-methyl-1-[3-(4-acetylphenyl)phenyl]-1,4-dihydro[1,8]naphthyridin-4-one-3-carboxamide;
N-Isopropyl-1-[3-(pyridin-3-yl)phenyl]-1,4-dihydro[1,8]naphthyridin-4-one-3-carboxamide;
N-Isopropyl-1-[3-(indol-5-yl)phenyl]-1,4-dihydro[1,8]naphthyridin-4-one-3-carboxamide;
N-tert-Butyl-1-[3-(4-acetylphenyl)phenyl]-1,4-dihydro[1,8]naphthyridin-4-one-3-carboxamide;
N-(2,6-Dichloropyridin-4-yl)-1-[3-(pyridin-3-yl)phenyl]-1,4-dihydro[1,8]naphthyridin-4-one-3-carboxamide;
N-Isopropyl-1-{3-[4-(4-tertbutyloxycarbonylpiperazin-1-yl)phenyl]-phenyl}-1,4-dihydro[1,8]naphthyridin-4-one-3-carboxamide;
N-Isopropyl-1-[3-(quinolin-3-yl)phenyl]-1,4-dihydro[1,8]naphthyridin-4-one-carboxamide;
N-Isopropyl-1-[3-(pyrimidin-5-yl) phenyl]-1,4-dihydro[1,8]naphthyridin-4-one-carboxamide;
N-Cyclopropyl-1-[3-(pyridin-3-yl)phenyl]-1,4-dihydro[1,8]naphthyridin-4-one-3-carboxamide;
N-Isopropyl-1-[3-(5-methylthiopyridin-3-yl)phenyl]-1,4-dihydro[1,8]naphthyridin-4-one-3-carboxamide;
N-Cyclopropyl-1-[3-(4-hydroxymethylphenyl)phenyl]-1,4-dihydro[1,8]naphthyridin-4-one-3-carboxamide;
N-Cyclopropyl-1-[3-(pyridin-4-yl)phenyl]-1,4-dihydro[1,8]naphthyridin-4-one-3-carboxamide;
N-Cyclopropyl-1-[3-(4-ethylthiophenyl)phenyl]-1,4-dihydro[1,8]naphthyridin-4-one-3-carboxamide;
N-Cyclopropyl-1-[3-(3-thienyl)phenyl]-1,4-dihydro[1,8]naphthyridin-4-one-3-carboxamide;
N-Cyclopropyl-1-[3-(4-sulfamoylphenyl)phenyl]-1,4-dihydro[1,8]naphthyridin-4-one-3-carboxamide;
N-Isopropyl-1-[3-(3-ethoxyphenyl)phenyl]-1,4-dihydro[1,8]naphthyridin-4-one-3-carboxamide;
N-Isopropyl-1-[3-(4-methylthiophenyl)phenyl]-1,4-dihydro[1,8]naphthyridin-4-one-3-carboxamide;
N-Isopropyl-1-[3-(3-acetyl-4-hydroxyphenyl)phenyl]-1,4-dihydro[1,8]naphthyridin-4-one-3-carboxamide;
N-Isopropyl-1-[3-(5-carboethoxypyridin-3-yl)phenyl]-1,4-dihydro[1,8]naphthyridin-4-one-3-carboxamide;
N-Isopropyl-1-{3-[5-(1-hydroxy-1-methylethyl)pyridin-3-yl]phenyl}-1,4-dihydro[1,8]naphthyridin-4-one-3-carboxamide;
N-Isopropyl-1-{3-[6-(2-methylpropyl)pyridin-3-yl]phenyl}-1,4-dihydro[1,8]naphthyridin-4-one-3-carboxamide;
N-Isopropyl-1-[3-(5-acetylpyridin-3-yl)phenyl]-1,4-dihydro[1,8]naphthyridin-4-one-3-carboxamide;
N-Isopropyl-1-[3-(6-methylpyridin-3-yl)phenyl]-1,4-dihydro[1,8]naphthyridin-4-one-3-carboxamide;
N-Cyclopropyl-1-[3-(1-oxidopyrimidin-5-yl) phenyl]-1,4-dihydro[1,8]naphthyridin-4-one-3-carboxamide;
1-{3-[6-(1-Hydroxy-1-methylethyl)-1-oxidopyridin-3-yl]phenyl}-1,4-dihydro[1,8]naphthyridin-4-one-3-carboxamide;
N-Isopropyl-1-{3-[4-(pyridin-3-yl)phenyl]phenyl}-1,4-dihydro[1,8]naphthyridin-4-one-3-carboxamide;
N-Cyclopropyl-1-[3-(5-methylsulfonylpyridin-3-yl)]phenyl]-1,4-dihydro[1,8]naphthyridin-4-one-3-carboxamide;
N-Cyclopropyl-1-{3-[4-(1-hydroxy-1-methylethyl)-1-oxidopyridin-2-yl]phenyl}-1,4-dihydro[1,8]naphthyridin-4-one-3-carboxamide;
N-Cyclopropyl-1-{3-[5-(1-hydroxy-1-methylethyl)pyridin-2-yl]phenyl}-1,4-dihydro[1,8]naphthyridin-4-one-3-carboxamide;
N-Cyclopropyl-1-{3-[3-(1-hydroxy-1-methylethyl)pyridin-4-yl]phenyl}-1,4-dihydro[1,8]naphthyridin-4-one-3-carboxamide;
N-Cyclopropyl-1-{3-[3-(1-hydroxy-1-methylethyl)-1-oxidopyridin-4-yl]phenyl}-1,4-dihydro[1,8]naphthyridin-4-one-3-carboxamide;
N-Cyclopropyl-1-[3-(6-isopropylsulfonylpyridin-3-yl)]phenyl]-1,4-dihydro[1,8]naphthyridin-4-one-3-carboxamide;
N-Cyclopropyl-1-[3-(6-methoxypyridin-3-yl)phenyl]-1,4-dihydro[1,8]naphthyridin-4-one-3-carboxamide;
N-Cyclopropyl-1-[3-(6-methylpyridin-3-yl)phenyl]-1,4-dihydro[1,8]naphthyridin-4-one-3-carboxamide;
N-Cyclopropyl-1-{3-[6-(2,2,2-trifluoroethoxy)pyridin-3-yl]phenyl}-1,4-dihydro[1,8]naphthyridin-4-one-3-carboxamide;
N-Cyclopropyl-1-[3-(5-bromopyridin-3-yl)phenyl]-1,4-dihydro[1,8]naphthyridin-4-one-3-carboxamide;
N-Cyclopropyl-1-[3-(6-benzyloxypyridin-3-yl)phenyl]-1,4-dihydro[1,8]naphthyridin-4-one-3-carboxamide;
N-Cyclopropyl-1-{3-[6-dicyclopropyl(hydroxy)methyl-1-oxidopyridin-3-yl]phenyl}-1,4-dihydro[1,8]naphthyridin-4-one-3-carboxamide;
N-Cyclopropyl-1-{3-[5-(1-hydroxy-1-methylethyl)-1-oxidopyridin-2-yl]phenyl}-1,4-dihydro[1,8]naphthyridin-4-one-3-carboxamide;
N-Cyclopropyl-1-{3-[6-(1-hydroxy-1-methylethyl)pyridin-3-yl]phenyl}-1,4-dihydro[1,8]naphthyridin-4-one-3-carboxamide;
N-Isobutyl-1-{3-[6-(1-hydroxy-1-methylethyl)pyridin-3-yl]phenyl}-1,4-dihydro[1,8]naphthyridin-4-one-3-carboxamide;
N-Cyclopropyl-1-{5-bromo-3-[6-(1-hydroxy-1-methylethyl)pyridin-3-yl]phenyl}-1,4-dihydro[1,8]naphthyridin-4-one-3-carboxamide;
N-Cyclopropyl-1-{3-[6-(1-hydroxy-1-methylethyl)pyridin-2-yl]phenyl}-1,4-dihydro[1,8]naphthyridin-4-one-3-carboxamide;
N-Isopropyl-1-[3-(4-methylsulfonylphenyl)phenyl]-1,4-dihydro[1,8]naphthyridin-4-one-3-carboxamide;
N-Cyclopropyl-1-[3-(6-methylsulfonylpyridin-3-yl)phenyl]-1,4-dihydro[1,8]naphthyridin-4-one-3-carboxamide;
N-Isopropyl-1-[3-(5-methylsulfonylpyridin-3-yl)phenyl]-1,4-dihydro[1,8]naphthyridin-4-one-3-carboxamide;
N-Cyclopropyl-1-[3-(4-ethylsulfonylphenyl)phenyl]-1,4-dihydro[1,8]naphthyridin-4-one-3-carboxamide;
N-Cyclopropyl-1-[3-(4-ethylsulfinylphenyl)phenyl]-1,4-dihydro[1,8]naphthyridin-4-one-3-carboxamide;
N-Isopropyl-1-{3-[4-(1-oximidoethyl)phenyl]phenyl}-1,4-dihydro[1,8]naphthyridin-4-one-3-carboxamide;
N-Isopropyl-1-{3-[4-(4-piperazin-1-yl)phenyl]-phenyl}-1,4-dihydro[1,8]naphthyridin-4-one-3-carboxamide;
N-Cyclopropyl-1-[3-(4-methylsulfonylmethylphenyl)phenyl]-1,4-dihydro[1,8]naphthyridin-4-one-3-carboxamide;
N-Cyclopropyl-1-[3-(1,6-dihydro-6-oxopyridin-3-yl)phenyl]-1,4-dihydro[1,8]naphthyridin-4-one-3-carboxamide;
N-Cyclopropyl-1-[[3-{5-[6-(1-hydroxy-1-methylethyl)pyridin-3-yl]pyridin-3-yl}phenyl]]-1,4-dihydro[1,8]naphthyridin-4-one-3-carboxamide;
N-Isopropyl-1-[3-(1-oxidopyridin-3-yl)phenyl]-1,4-dihydro[1,8]naphthyridin-4-one-3-carboxamide;
N-(2,6-Dichloropyridin-4-yl)-1-[3-(1-oxidopyridin-3-yl)phenyl]-1,4-dihydro[1,8]naphthyridin-4-one-3-carboxamide;
N-Isopropyl-1-[3-(5-carboethoxy-1-oxidopyridin-3-yl)phenyl]-1,4-dihydro[1,8]naphthyridin-4-one-3-carboxamide;
N-Isopropyl-1-{3-[5-(1-hydroxy-1-methylethyl)-1-oxidopyridin-3-yl]phenyl}-1,4-dihydro[1,8]naphthyridin-4-one-3-carboxamide;
N-Isopropyl-1-{3-[6-(2-methylpropyl)-1-oxidopyridin-3-yl]phenyl}-1,4-dihydro[1,8]naphthyridin-4-one-3-carboxamide;
N-Isopropyl-1-[3-(6-methyl-1-oxidopyridin-3-yl)phenyl]-1,4-dihydro[1,8]naphthyridin-4-one-3-carboxamide;
N-Cyclopropyl-1-[3-(1-oxidopyridin-3-yl)phenyl]-1,4-dihydro[1,8]naphthyridin-4-one-3-carboxamide;
N-Cyclopropyl-1-[3-[6-(1-hydroxy-1-methylethyl)-1-oxidopyridin-3-yl]phenyl)-1,4-dihydro[1,8]naphthyridin-4-one-3-carboxamide;
N-Cyclopropyl-1-[3-(1-oxidopyridin-4-yl)phenyl]-1,4-dihydro[1,8]naphthyridin-4-one-3-carboxamide;
N-Cyclopropyl-1-[3-(5-bromo-1-oxidopyridin-3-yl)phenyl]-1,4-dihydro[1,8]naphthyridin-4-one-3-carboxamide;
N-Cyclopropyl-1-[[3-{5-[6-(1-hydroxy-1-methylethyl)-1-oxidopyridin-3-yl]pyridin-3-yl}phenyl]]-1,4-dihydro[1,8]naphthyridin-4-one-3-carboxamide;
N-Cyclopropyl-1-[[3-{5-[6-(1-hydroxy-1-methylethyl)pyridin-3-yl]-1-oxidopyridin-3-yl}phenyl]]-1,4-dihydro[1,8]naphthyridin-4-one-3-carboxamide;
N-Cyclopropyl-1-[[3-{5-[6-(1-hydroxy-1-methylethyl)-1-oxidopyridin-3-yl]-1-oxidopyridin-3-yl}phenyl]]-1,4-dihydro[1,8]naphthyridin-4-one-3-carboxamide;
N-Isopropyl-1-[3-(1-oxidoquinolin-3-yl)phenyl]-1,4-dihydro[1,8]naphthyridin-4-one-carboxamide;
N-Isobutyl-1-{3-[6-(1-hydroxy-1-methylethyl)-1-oxidopyridin-3-yl]phenyl}-1,4-dihydro[1,8]naphthyridin-4-one-3-carboxamide;
N-Cyclopropyl-1-[3-(6-methyl-1-oxidopyridin-3-yl)]phenyl]-1,4-dihydro[1,8]naphthyridin-4-one-3-carboxamide;
N-Cyclopropyl-1-[3-(6-methylsulfonyl-1 oxidopyridin-3-yl)phenyl]-1,4-dihydro[1,8]naphthyridin-4-one-3-carboxamide;
N-Cyclopropyl-1-{5-bromo-3-[6-(1-hydroxy-1-methylethyl)-1-oxidopyridin-3-yl]phenyl}-1,4-dihydro[1,8]naphthyridin-4-one-3-carboxamide;
N-Cyclopropyl-1-{3-[6-(1,2-dihydroxy-1-methylethyl)-1-oxidopyridin-3-yl]phenyl}-1,4-dihydro[1,8]naphthyridin-4-one-3-carboxamide.
N-Isopropyl-1-[3-(phenylethynyl)phenyl]-1,4-dihydro[1,8]naphthyridin-4-one-3-carboxamide;
N-Isopropyl-1-[3-(2-pyridinylethynyl)phenyl]-1,4-dihydro[1,8]naphthyridin-4-one-3-carboxamide;
N-Isopropyl-1-[3-(4-pyridinylethynyl)phenyl]-1,4-dihydro[1,8]naphthyridin-4-one-3-carboxamide;
N-Isopropyl-1-[3-(1-oxido-4-pyridinylethynyl)phenyl]-1,4-dihydro[1,8]naphthyridin-4-one-3-carboxamide;
N-Isopropyl-1-(3-ethynylphenyl)-1,4-dihydro[1,8]naphthyridin-4-one-3-carboxamide;
N-Cyclopropyl-1-(3-ethynylphenyl)-1,4-dihydro[1,8]naphthyridin-4-one-3-carboxamide;
N-Isopropyl-1-[3-(3-pyridinylethynyl)phenyl]-1,4-dihydro[1,8]naphthyridin-4-one-3-carboxamide;
N-Isopropyl-1-[3-(1-oxido-3-pyridinylethynyl)phenyl]-1,4-dihydro[1,8]naphthyridin-4-one-3-carboxamide;
N-Cyclopropyl-1-[3-(3-pyridinylethynyl)phenyl]-1,4-dihydro[1,8]naphthyridin-4-one-3-carboxamide;
N-Isopropyl-1-[3-(3-hydroxy-3-methylbut-1-ynyl)phenyl]-1,4-dihydro[1,8]naphthyridin-4-one-3-carboxamide;
N-Cyclopropyl-1-[3-(3-hydroxy-3-methylbut-1-ynyl)phenyl]-1,4-dihydro[1,8]naphthyridin-4-one-3-carboxamide;
N-Isopropyl-1-[3-(1-hydroxycyclopentyl)ethynylphenyl]-1,4-dihydro[1,8]naphthyridin-4-one-3-carboxamide;
N-Isopropyl-1-[3-(1-hydroxycyclopropyl)ethynylphenyl]-1,4-dihydro[1,8]naphthyridin-4-one-3-carboxamide;
N-Isopropyl-1-{3-[4,4,4-trifluoro-3-hydroxy-3-(trifluoromethyl)but-1-ynyl]phenyl}-1,4-dihydro[1,8]naphthyridin-4-one-3-carboxamide;
N-Isopropyl-1-[3-(3-hydroxy-3-phenylbut-1-ynyl)phenyl]-1,4-dihydro[1,8]naphthyridin-4-one-3-carboxamide;
N-Cyclopropyl-1-[3-(1-oxido-3-pyridinylethynyl)phenyl]-1,4-dihydro[1,8]naphthyridin-4-one-3-carboxamide;
N-Isopropyl-1-[3-(3-amino-3-ethylpent-1-ynyl)phenyl]-1,4-dihydro[1,8]naphthyridin-4-one-3-carboxamide; or
N-Cyclopropyl-1-[3-(3-amino-3-ethylpent-1-ynyl)phenyl]-1,4-dihydro[1,8]naphthyridin-4-one-3-carboxamide;
N-Isopropyl-1-[3-(3-quinolinylethynyl)phenyl]-1,4-dihydro[1,8]naphthyridin-4-one-3-carboxamide; or
N-Isopropyl-1-[3-(1-oxido-3-quinolinylethynyl)phenyl]-1,4-dihydro[1,8]naphthyridin-4-one-3-carboxamide;
N-Isopropyl-1-[3-(cyclopropylethynyl)phenyl]-1,4-dihydro[1,8]naphthyridin-4-one-3-carboxamide;
N-Isopropyl-1-[3-(6-amino-3-pyridinylethynyl)phenyl]-1,4-dihydro[1,8]naphthyridin-4-one-3-carboxamide;
N-Isopropyl-1-{3-[5-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[6-(1-hydroxy-1-methylethyl)-3-pyridinylethynyl]phenyl}-1,4-dihydro[1,8]naphthyridin-4-one-3-carboxamide;
N-Isopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[4-(1-hydroxy-1-methylethyl)-2-pyridinylethynyl]phenyl}-1,4-dihydro[1,8]naphthyridin-4-one-3-carboxamide;
N-Isopropyl-1-{3-[5-(1-hydroxy-1-methylethyl)-2-pyridinylethynyl]phenyl}-1,4-dihydro[1,8]naphthyridin-4-one-3-carboxamide;
N-Isopropyl-1-{3-[6-(1-hydroxy-1-methylethyl)-2-pyridinylethynyl]phenyl}-1,4-dihydro[1,8]naphthyridin-4-one-3-carboxamide;
N-Cyclopropyl-1-{3-[6-(1-hydroxy-1-methylethyl)-1-oxido-3-pyridinylethynyl]phenyl}-1,4-dihydro[1,8]naphthyridin-4-one-3-carboxamide;
N-Isopropyl-1-{3-[(4-pyridin-3-ylphenyl)ethynyl]phenyl}-1,4-dihydro[1,8]naphthyridin-4-one-3-carboxamide;
N-Isopropyl-1-(3-{[5-(1-hydroxy-1-methylethyl)thien-2-yl]ethynyl}phenyl)-1,4-dihydro[1,8]naphthyridin-4-one-3-carboxamide;
N-Isopropyl-1-(3-{[2-(1-hydroxy-1-methylethyl)-1,3-thiazol-5-yl]ethynyl}phenyl)-1,4-dihydro[1,8]naphthyridin-4-one-3-carboxamide;
1-[3-(1-Oxido-3-pyridinylethynyl)phenyl]-1,4-dihydro[1,8]naphthyridin-4-one-3-carboxamide; or
1-[3-(1-Oxido-3-pyridinylethynyl)phenyl]-1,4-dihydro[1,8]naphthyridin-4-one-3-carboxylic acid;
or a pharmaceutically acceptable salt thereof.

2. A method of enhancing cognition in a healthy subject according to claim 1 comprising administering a safe, non-emetic, cognition enhancing amount of compound.

3. A method of enhancing cognition in a healthy subject according to claim 2, wherein the healthy subject is a human 40 years of age or older.

4. A method of enhancing cognition in a healthy subject according to claim 2, wherein the healthy subject is a human 55 years of age or older.

5. A method of enhancing cognition in a healthy subject comprising administering a safe cognition enhancing amount of compound selected from the group consisting of: 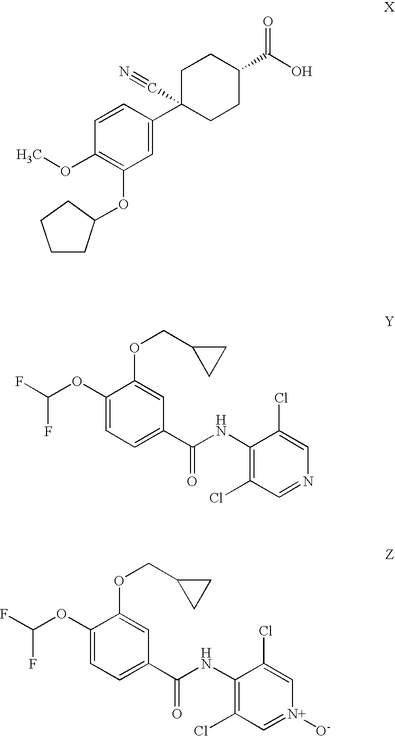
or a pharmaceutically acceptable salt thereof.

6. A method of enhancing cognition in a healthy subject according to claim 1 comprising administering a safe, non-emetic, cognition enhancing amount of compound.

7. A method of enhancing cognition in a healthy subject according to claim 2, wherein the healthy subject is a human 40 years of age or older.

8. A method of enhancing cognition in a healthy subject according to claim 2, wherein the healthy subject is a human 55 years of age or older.

9-11. (canceled)
